(19) United States
(12) Patent Application Publication
(10) Pub. No.: US 2023/0167450 A1
(43) Pub. Date: Jun. 1, 2023

(54) BIFUNCTIONAL MOLECULES AND METHODS OF USING THEREOF

(71) Applicant: Flagship Pioneering, Inc., Cambridge, MA (US)

(72) Inventors: Nathan Wilson STEBBINS, Cambridge, MA (US); Benjamin Andrew PORTNEY, Cambridge, MA (US); Eric Bruno VALEUR, Cambridge, MA (US); Jacob Rosenblum RUBENS, Cambridge, MA (US); Kaveh DANESHVAR, Cambridge, CA (US); Alexandra Rachael SNEIDER, Cambridge, MA (US); Mitchell GUTTMAN, Los Angeles, CA (US)

(73) Assignee: Flagship Pioneering, Inc., Cambridge, MA (US)

(21) Appl. No.: 17/920,752

(22) PCT Filed: Apr. 21, 2021
(86) PCT No.: PCT/US2021/028498

§ 371 Date: Oct. 21, 2022

Related U.S. Application Data
(60) Provisional application No. 63/013,462, filed on Apr. 21, 2020.
(60) Provisional application No. 63/139,916, filed on Jan. 21, 2021.

(51) Int. Cl. C12N 15/113; C12N 15/115

(52) U.S. Cl. CPC C12N 15/1137; C12N 15/115; C12N 2310/11; C12N 2310/16; C12N 2310/3519; C12N 2310/3231

(57) ABSTRACT

The present disclosure relates generally to compositions of synthetic bifunctional molecules comprising a first domain that specifically binds to a target ribonucleic acid and a second domain that specifically binds to a target protein, and uses thereof.

### BACKGROUND

The modulation of RNA translation plays a fundamental role in moderating cellular events and in the response to disease states within organisms, at the cellular as well as the tissue level. Some disease states can be ameliorated when the expression of one or more proteins is increased, which can be achieved by increasing RNA translation.

A binding specificity between a target RNA and protein may provide tools to effectively deliver molecules to increase mRNA translation of a specific target.

### SUMMARY

In one aspect, the present disclosure provides a method of increasing translation of a target ribonucleic acid (RNA) in a cell comprising: administering to the cell a synthetic bifunctional molecule comprising: a first domain comprising an antisense oligonucleotide (ASO) or a first small molecule, wherein the first domain specifically binds to an RNA sequence of the target RNA; and a second domain comprising a second small molecule or an aptamer, wherein the second domain specifically binds to a target polypeptide. In some embodiments, the synthetic bifunctional molecule further comprises a linker that conjugates the first domain and the second domain. In some embodiments, the target polypeptide directly or indirectly promotes, boosts, or increases translation of the target RNA in the cell. In some embodiments, the target polypeptide is a target protein.

In some embodiments, the first domain comprises the ASO. In some embodiments, the first domain is an ASO. In some embodiments, the ASO comprises one or more locked nucleotides, one or more modified nucleobases, or a combination thereof. In some embodiments, the ASO comprises a 5′ locked terminal nucleotide, a 3′ locked terminal nucleotide, or a 5′ and a 3′ locked terminal nucleotide. In some embodiments, the ASO comprises a locked nucleotide at an internal position in the ASO. In some embodiments, the ASO comprises a sequence comprising 30% to 60% GC content. In some embodiments, the ASO comprises a length of 8 to 30 nucleotides. In some embodiments, the ASO comprises a length from 12 to 25 nucleotides. In some embodiments, the ASO comprises a length from 14 to 24 nucleotides. In some embodiments, the ASO comprises a length from 16 to 20 nucleotides. In some embodiments, the ASO binds to Renilla Luciferase (Rluc) RNA. In some embodiments, the linker is conjugated at a 5′ end or a 3′ end of the ASO.

In some embodiments, the cell is a human cell. In some embodiments, the human cell is infected with a virus. In some embodiments, the human cell is a cancer cell. In some embodiments, the cell is a bacterial cell.

In some embodiments, the first domain comprises a small molecule. In some embodiments, the small molecule is selected from the group consisting of Table 2. In some embodiments, the second domain comprises a small molecule. In some embodiments, the small molecule is an organic compound having a molecular weight of 900 daltons or less. In some embodiments, the second small molecule comprises Ibrutinib or Ibrutinib-MPEA.

In some embodiments, the second domain is an aptamer. In some embodiments, the linker comprises:

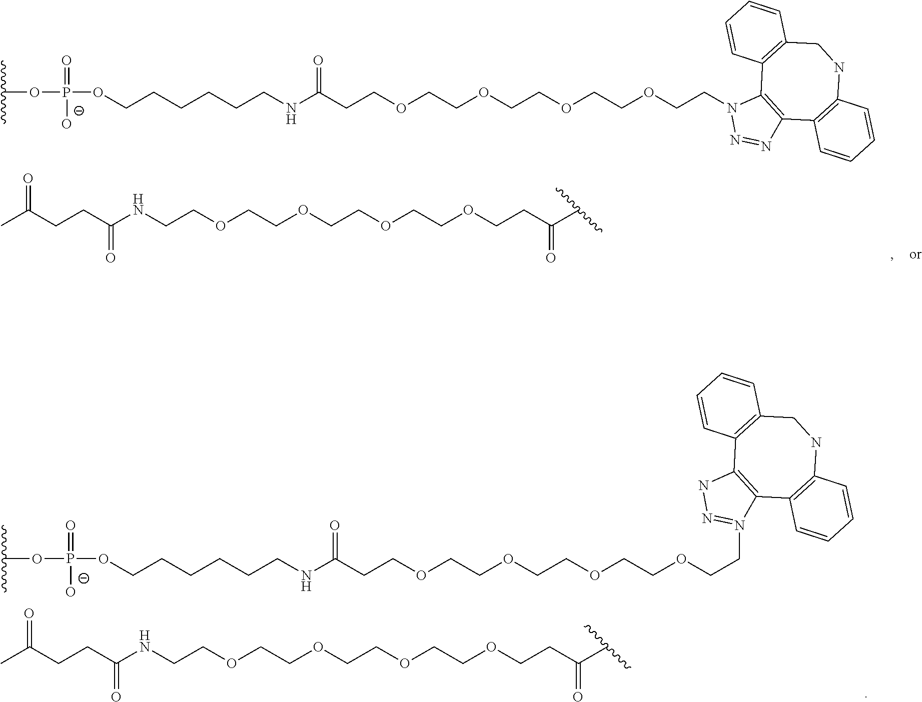

In some embodiments, the target ribonucleic acid sequence is a nuclear RNA or a cytoplasmic RNA. In some embodiments, the nuclear RNA or the cytoplasmic RNA is a long noncoding RNA (lncRNA), pre-mRNA, mRNA, microRNA, enhancer RNA, transcribed RNA, nascent RNA, chromosome-enriched RNA, ribosomal RNA, membrane enriched RNA, or mitochondrial RNA. In some embodiments, a subcellular localization of the target RNAis selected from the group consisting of nucleus, Golgi, endoplasmic reticulum, vacuole, lysosome, and mitochondrion. In some embodiments, the target RNA is located in an intron, an exon, a 5′ UTR, or a 3′ UTR of the target RNA.

In some embodiments, the target polypeptide comprises EIF4E. In some embodiments, the target polypeptide comprises YTHDF1. In some embodiments, the target polypeptide is endogenous. In some embodiments, the target polypeptide is intracellular. In some embodiments, the target polypeptide is an enzyme, a scaffolding protein, or a regulatory protein. In some embodiments, the ribonucleic acid is associated with a disease or disorder.

In some embodiments the target polypeptide is an exogenous. In some embodiments the target polypeptide is a fusion protein or recombinant protein.

In some embodiments, the second domain specifically binds to an active site or an allosteric site on the target polypeptide. In some embodiments, binding of the second domain to the target polypeptide is noncovalent or covalent. In some embodiments, binding of the second domain to the target polypeptide is covalent and reversible or covalent and irreversible.

In some embodiments, the target RNA is in a transcript of a gene selected from Table 3 or Table 4. In some embodiments, the target RNA is associated with a disease or disorder. In some embodiments, the target RNA is associated with a disease from Table 4. In some embodiments, the disease is any disorder caused by an organism. In some embodiments, the organism is a prion, a bacteria, a virus, a fungus, or a parasite. In some embodiments, the disease or disorder is a cancer, a metabolic disease, an inflammatory disease, an autoimmune disease, a cardiovascular disease, an infectious disease, a genetic disease, or a neurological disease. In some embodiments, the disease is a cancer and wherein the target gene is an oncogene. In some embodiments, the second domain specifically binds to a protein-RNA interaction domain, and the RNA of the protein-RNA interaction is associated with a gene selected from Table 3 or Table 4. In some embodiments, the protein-RNA interaction blocks an effector protein from binding to the RNA sequence. In some embodiments, the protein-RNA interaction is associated with a disease or disorder. In some embodiments, the disease is any disorder caused by an organism. In some embodiments, the organism is a prion, a bacteria, a virus, a fungus, or a parasite. In some embodiments, the disease or disorder is a cancer, a metabolic disease, an inflammatory disease, an autoimmune disease, a cardiovascular disease, an infectious disease, a genetic disease, or a neurological disease. In some embodiments, the disease is a cancer and wherein the target gene is an oncogene.

In some aspect, the present disclosure also provides a synthetic bifunctional molecule for increasing translation of a target ribonucleic acid (RNA) in a cell, the synthetic bifunctional molecule comprising: a first domain comprising a first small molecule or an antisense oligonucleotide (ASO), wherein the first domain specifically binds to an RNA sequence of the target RNA; and a second domain comprising a second small molecule or an aptamer, wherein the second domain specifically binds to a target polypeptide. In some embodiments, the first domain and the second domain are those described above. In some embodiments, the synthetic bifunctional molecule comprises a linker that conjugates the first domain to the second domain. In some embodiments, the target polypeptide directly or indirectly promotes, boosts, or increases translation of the target RNA in the cell. In some embodiments, the target polypeptide is a target protein. In some embodiments, the linker comprises:

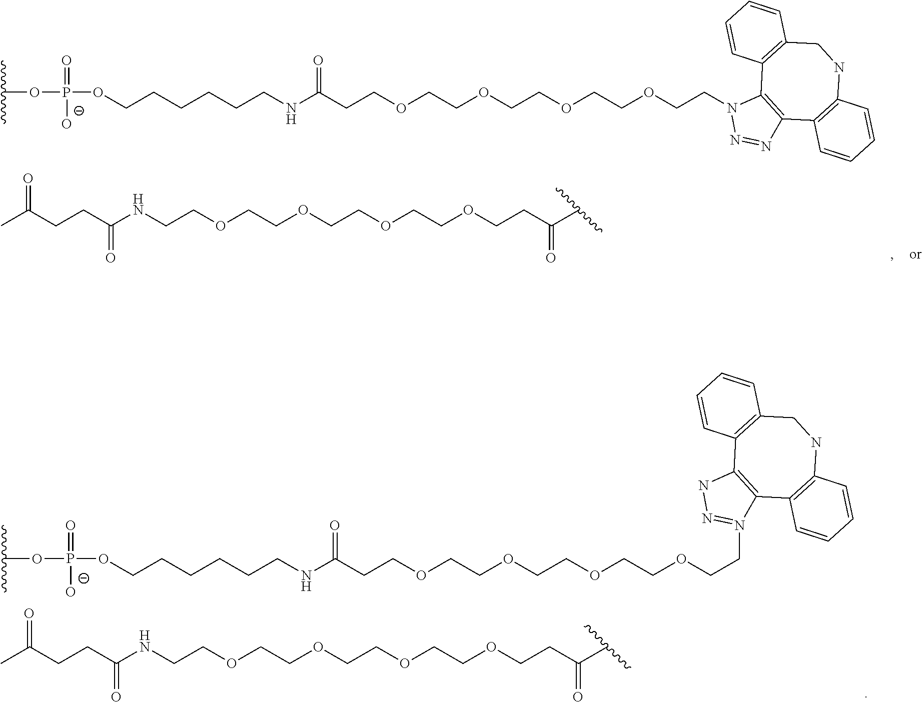

In some embodiments, the linker includes a mixer of regioisomers. In some embodiments, the mixer of regioisomers is Linker 2 described herein. In some embodiments, the target polypeptide comprises EIF4E. In some embodiments, the target polypeptide comprises YTHDF1.

### BRIEF DESCRIPTION OF THE DRAWINGS

The following detailed description of the embodiments of the present disclosure will be better understood when read in conjunction with the appended drawings. For the purpose of illustrating the present disclosure, they are shown in the drawings embodiments, which are presently exemplified. It should be understood, however, that the present disclosure is not limited to the precise arrangement and instrumentalities of the embodiments shown in the drawings.

FIG. 1 depicts mass spectrometry data identifying fractions containing free oligonucleotide and oligonucleotide conjugated to small molecule.

FIG. 2A shows a scheme to form an exemplary ternary complex. As evidence of ternary complex formation (Target RNA-bifunctional molecule-effector protein) in vitro, FIG. 2B depicts results from gel analysis that detects formation of ternary complex by shift in gel.

FIG. 3 is an image showing that the conjugate of Ibrutinib and an ASO, an exemplary embodiment of the bifunctional molecules as provided herein, forms a tertiary complex with Bruton's Tyrosine Kinase (BTK) via Ibrutinib and the Cy5-labeled IVT RNA via the ASO, respectively.

FIG. 4 shows enhancing the translation of an RNA by bifunctional molecules and a BTK-YTHDF1 effector protein.

FIG. 5 shows enhancing the translation of an RNA by bifunctional molecules and a BTK-EIF4E effector protein.

### DETAILED DESCRIPTION

The present disclosure generally relates to bifunctional molecules. Generally, the bifunctional molecules are designed and synthesized to bind to two or more unique targets. A first target can be a nucleic acid sequence, for example an RNA. A second target can be a protein, peptide, or other effector molecule. The bifunctional molecules described herein comprise a first domain that specifically binds to a target nucleic acid sequence or structure (e.g., a target RNA sequence) and a second domain that specifically binds to a target polypeptide or protein. Bifunctional molecule compositions, preparations of compositions thereof and uses thereof are also described.

The present disclosure is described with respect to particular embodiments and with reference to certain figures but the present disclosure is not limited thereto but only by the claims. Terms as set forth hereinafter are generally to be understood in their common sense unless indicated otherwise.

The synthetic bifunctional molecules comprising a first domain that specifically binds to an RNA sequence of a target RNA and a second domain that specifically binds to a target polypeptide or protein, compositions comprising such bifunctional molecules, methods of using such bifunctional molecules, etc. as described herein are based in part on the examples which illustrate how the bifunctional molecules comprising different components, for example, unique sequences, different lengths, and modified nucleotides (e.g., locked nucleotides), be used to achieve different technical effects (e.g., translation increase of a target RNA in a cell). It is on the basis of inter alia these examples that the description hereinafter contemplates various variations of the specific findings and combinations considered in the examples.

### Bifunctional Molecule

In some aspects, the present disclosure relates to a bifunctional molecule comprising a first domain that binds to a target nucleic acid sequence (e.g., an RNA sequence) and a second domain that binds to a target polypeptide or protein. The bifunctional molecules described herein are designed and synthesized so that a first domain is conjugated to a second domain.

### First Domain

The bifunctional molecule as described herein comprise a first domain that specifically binds to a target nucleic acid sequence or structure (e.g., an RNA sequence). In some embodiments, the first domain comprises a small molecule or an antisense oligonucleotide (ASO).

### Antisense Oligonucleotide (ASO)

In some embodiments, the first domain of the bifunctional molecule as described herein, which specifically binds to an RNA sequence of a target RNA, is an ASO.

Routine methods can be used to design a nucleic acid that binds to the target sequence with sufficient specificity. As used herein, the terms “nucleotide,” “oligonucleotide,” and “nucleic acid” are used interchangeably. In some embodiments, the methods include using bioinformatics methods known in the art to identify regions of secondary structure. As used herein, the term “secondary structure” refers to the basepairing interactions within a single nucleic acid polymer or between two polymers. For example, the secondary structures of RNA include, but are not limited to, a double-stranded segment, bulge, internal loop, stem-loop structure (hairpin), two-stem junction (coaxial stack), pseudoknot, g-quadruplex, quasi-helical structure, and kissing hairpins. For example, “gene walk” methods can be used to optimize the activity of the nucleic acid; for example, a series of oligonucleotides of 10-30 nucleotides spanning the length of a target RNA or a gene can be prepared, followed by testing for activity. Optionally, gaps, e.g., of 5-10 nucleotides or more, can be left between the target sequences to reduce the number of oligonucleotides synthesized and tested.

Once one or more target regions, segments or sites have been identified, e.g., within a sequence of interest, nucleotide sequences are chosen that are sufficiently complementary to the target, i.e., that hybridize sufficiently well and with sufficient specificity (i.e., do not substantially bind to other non-target RNAs), to give the desired effect, e.g., binding to the RNA.

As described herein, hybridization means hydrogen bonding, which may be Watson-Crick, Hoogsteen or reversed Hoogsteen hydrogen bonding, between complementary nucleoside or nucleotide bases. For example, adenine and thymine are complementary nucleobases which pair through the formation of hydrogen bonds. Complementary, as used herein, refers to the capacity for precise pairing between two nucleotides. For example, if a nucleotide at a certain position of an oligonucleotide is capable of hydrogen bonding with a nucleotide at the same position of an RNA molecule, then the ASO and the RNA are considered to be complementary to each other at that position. The ASO and the RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the ASO and the RNA target. For example, if a base at one position of the ASO is capable of hydrogen bonding with a base at the corresponding position of an RNA, then the bases are considered to be complementary to each other at that position. 100% complementarity is not required.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule or the target gene elicit the desired effects as described herein, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency.

In general, the ASO useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Percent complementarity of an ASO with a region of a target nucleic acid can be determined routinely using basic local alignment search tools (BLAST programs) (Altschul et al, J. Mol. Biol., 1990, 215, 403-410; Zhang and Madden, Genome Res., 1997, 7, 649-656). The ASO that hybridizes to an RNA can be identified through routine experimentation. In general, the ASO must retain specificity for their target, i.e., must not directly bind to other than the intended target.

In certain embodiments, the ASO described herein comprises modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, one nucleoside comprising a modified nucleobase is in the central region of a modified oligonucleotide. In certain such embodiments, the sugar moiety of said nucleoside is a 2′-p-D-deoxyribosyl moiety. In certain such embodiments, the modified nucleobase is selected from: 5-methyl cytosine, 2-thiopyrimidine, 2-thiothymine, 6-methyladenine, inosine, pseudouracil, or 5-propynepyrimidine.

In certain embodiments, the ASO described herein comprises modified and/or unmodified internucleoside linkages arranged along the oligonucleotide or region thereof in a defined pattern or motif. In certain embodiments, each internucleoside linkage is a phosphodiester internucleoside linkage (P═O). In certain embodiments, each internucleoside linkage of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate. In certain embodiments, the internucleoside linkages within the central region of a modified oligonucleotide are all modified. In certain such embodiments, some or all of the internucleoside linkages in the 5′-region and 3′-region are unmodified phosphate linkages. In certain embodiments, the terminal internucleoside linkages are modified. In certain embodiments, the internucleoside linkage motif comprises at least one phosphodiester internucleoside linkage in at least one of the 5′-region and the 3′-region, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the 5′-region and 3′-region are (Sp) phosphorothioates, and the central region comprises at least one Sp, Sp, Rp motif. In certain embodiments, populations of modified oligonucleotides are enriched for modified oligonucleotides comprising such internucleoside linkage motifs.

In certain embodiments, the ASO comprises a region having an alternating internucleoside linkage motif. In certain embodiments, oligonucleotides comprise a region of uniformly modified internucleoside linkages. In certain such embodiments, the internucleoside linkages are phosphorothioate internucleoside linkages. In certain embodiments, all of the internucleoside linkages of the oligonucleotide are phosphorothioate internucleoside linkages. In certain embodiments, each internucleoside linkage of the oligonucleotide is selected from phosphodiester or phosphate and phosphorothioate. In certain embodiments, each internucleoside linkage of the oligonucleotide is selected from phosphodiester or phosphate and phosphorothioate and at least one internucleoside linkage is phosphorothioate.

In certain embodiments, ASO comprises at least 6 phosphorothioate internucleoside linkages. In certain embodiments, the oligonucleotide comprises at least 8 phosphorothioate internucleoside linkages. In certain embodiments, the oligonucleotide comprises at least 10 phosphorothioate internucleoside linkages. In certain embodiments, the oligonucleotide comprises at least one block of at least 6 consecutive phosphorothioate internucleoside linkages. In certain embodiments, the oligonucleotide comprises at least one block of at least 8 consecutive phosphorothioate internucleoside linkages. In certain embodiments, the oligonucleotide comprises at least one block of at least 10 consecutive phosphorothioate internucleoside linkages. In certain embodiments, the oligonucleotide comprises at least block of at least one 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide.

In certain embodiments, the ASO comprises one or more methylphosphonate linkages. In certain embodiments, modified oligonucleotides comprise a linkage motif comprising all phosphorothioate linkages except for one or two methylphosphonate linkages. In certain embodiments, one methylphosphonate linkage is in the central region of an oligonucleotide.

In certain embodiments, it is desirable to arrange the number of phosphorothioate internucleoside linkages and phosphodiester internucleoside linkages to maintain nuclease resistance. In certain embodiments, it is desirable to arrange the number and position of phosphorothioate internucleoside linkages and the number and position of phosphodiester internucleoside linkages to maintain nuclease resistance. In certain embodiments, the number of phosphorothioate internucleoside linkages may be decreased and the number of phosphodiester internucleoside linkages may be increased. In certain embodiments, the number of phosphorothioate internucleoside linkages may be decreased and the number of phosphodiester internucleoside linkages may be increased while still maintaining nuclease resistance. In certain embodiments it is desirable to decrease the number of phosphorothioate internucleoside linkages while retaining nuclease resistance. In certain embodiments it is desirable to increase the number of phosphodiester internucleoside linkages while retaining nuclease resistance.

The ASOs described herein can be short or long. The ASOs may be from 8 to 200 nucleotides in length, in some instances between 10 and 100, in some instances between 12 and 50. In some embodiments, the ASO comprises the length of from 8 to 30 nucleotides. In some embodiments, the ASO comprises the length of from 9 to 30 nucleotides. In some embodiments, the ASO comprises the length of from 10 to 30 nucleotides. In some embodiments, the ASO comprises the length of from 11 to 30 nucleotides. In some embodiments, the ASO comprises the length of from 12 to 30 nucleotides. In some embodiments, the ASO comprises the length of from 13 to 30 nucleotides. In some embodiments, the ASO comprises the length of from 14 to 30 nucleotides. In some embodiments, the ASO comprises the length of from 15 to 30 nucleotides. In some embodiments, the ASO comprises the length of from 16 to 30 nucleotides. In some embodiments, the ASO comprises the length of from 17 to 30 nucleotides. In some embodiments, the ASO comprises the length of from 18 to 30 nucleotides. In some embodiments, the ASO comprises the length of from 19 to 30 nucleotides. In some embodiments, the ASO comprises the length of from 20 to 30 nucleotides.

In some embodiments, the ASO comprises the length of from 8 to 29 nucleotides. In some embodiments, the ASO comprises the length of from 9 to 29 nucleotides. In some embodiments, the ASO comprises the length of from 10 to 28 nucleotides. In some embodiments, the ASO comprises the length of from 11 to 28 nucleotides. In some embodiments, the ASO comprises the length of from 12 to 28 nucleotides. In some embodiments, the ASO comprises the length of from 13 to 28 nucleotides. In some embodiments, the ASO comprises the length of from 14 to 28 nucleotides. In some embodiments, the ASO comprises the length of from 15 to 28 nucleotides. In some embodiments, the ASO comprises the length of from 16 to 28 nucleotides. In some embodiments, the ASO comprises the length of from 17 to 28 nucleotides. In some embodiments, the ASO comprises the length of from 18 to 28 nucleotides. In some embodiments, the ASO comprises the length of from 19 to 28 nucleotides. In some embodiments, the ASO comprises the length of from 20 to 28 nucleotides.

In some embodiments, the ASO comprises the length of from 8 to 27 nucleotides. In some embodiments, the ASO comprises the length of from 9 to 27 nucleotides. In some embodiments, the ASO comprises the length of from 10 to 26 nucleotides. In some embodiments, the ASO comprises the length of from 10 to 25 nucleotides. In some embodiments, the ASO comprises the length of from 10 to 24 nucleotides. In some embodiments, the ASO comprises the length of from 11 to 24 nucleotides. In some embodiments, the ASO comprises the length of from 12 to 24 nucleotides. In some embodiments, the ASO comprises the length of from 13 to 24 nucleotides. In some embodiments, the ASO comprises the length of from 14 to 24 nucleotides. In some embodiments, the ASO comprises the length of from 15 to 24 nucleotides. In some embodiments, the ASO comprises the length of from 16 to 24 nucleotides. In some embodiments, the ASO comprises the length of from 17 to 28 nucleotides. In some embodiments, the ASO comprises the length of from 18 to 24 nucleotides. In some embodiments, the ASO comprises the length of from 19 to 24 nucleotides. In some embodiments, the ASO comprises the length of from 20 to 24 nucleotides.

In some embodiments, the ASO comprises the length of from 10 to 27 nucleotides. In some embodiments, the ASO comprises the length of from 11 to 26 nucleotides. In some embodiments, the ASO comprises the length of from 12 to 25 nucleotides. In some embodiments, the ASO comprises the length of from 12 to 24 nucleotides. In some embodiments, the ASO comprises the length of from 12 to 23 nucleotides. In some embodiments, the ASO comprises the length of from 12 to 22 nucleotides. In some embodiments, the ASO comprises the length of from 12 to 21 nucleotides. In some embodiments, the ASO comprises the length of from 12 to 20 nucleotides.

In some embodiments, the ASO comprises the length of from 16 to 27 nucleotides. In some embodiments, the ASO comprises the length of from 16 to 26 nucleotides. In some embodiments, the ASO comprises the length of from 16 to 25 nucleotides. In some embodiments, the ASO comprises the length of from 16 to 24 nucleotides. In some embodiments, the ASO comprises the length of from 16 to 23 nucleotides. In some embodiments, the ASO comprises the length of from 16 to 22 nucleotides. In some embodiments, the ASO comprises the length of from 16 to 21 nucleotides. In some embodiments, the ASO comprises the length of from 16 to 20 nucleotides. In some embodiments, the ASO comprises the length of 8, 9, 10, 11, 12, 13, 14, 15, 16, 17, 18, 19, 20, 21, 22, 23, 24, 25, 26, 27, 28, 29 or more nucleotides, and 30, 29, 28, 27, 26, 25, 24, 23, 22, 21, 20, 19, 18, 17, 16, 15, 14, 13, 12, 11, 10, 9 or fewer nucleotides.

As used herein, the term “GC content” or “guanine-cytosine content” refers to the percentage of nitrogenous bases in a DNA or RNA molecule that are either guanine (G) or cytosine (C). This measure indicates the proportion of G and C bases out of an implied four total bases, also including adenine and thymine in DNA and adenine and uracil in RNA. In some embodiments, the ASO comprises a sequence comprising from 30% to 60% GC content. In some embodiments, the ASO comprises a sequence comprising from 35% to 60% GC content. In some embodiments, the ASO comprises a sequence comprising from 40% to 60% GC content. In some embodiments, the ASO comprises a sequence comprising from 45% to 60% GC content. In some embodiments, the ASO comprises a sequence comprising from 50% to 60% GC content. In some embodiments, the ASO comprises a sequence comprising from 30% to 55% GC content. In some embodiments, the ASO comprises a sequence comprising from 30% to 50% GC content. In some embodiments, the ASO comprises a sequence comprising from 30% to 45% GC content. In some embodiments, the ASO comprises a sequence comprising from 30% to 40% GC content. In some embodiments, the ASO comprises a sequence comprising 30, 31, 32, 33, 34, 35, 36, 37, 38, 39, 40, 41, 42, 43, 44, 45, 46, 47, 48, 49, 50, 51, 52, 53, 54, 55, 56, 57, 58, 59% or more and 60, 59, 58, 57, 56, 55, 54, 53, 52, 51, 50, 49, 48, 47, 46, 45, 44, 43, 42, 41, 40, 39, 38, 37, 36, 35, 34, 33, 32, 31% or less GC content.

In some embodiments, the nucleotide comprises at least one or more of: a length of from 10 to 30 nucleotides; a sequence comprising from 30% to 60% GC content; and at least one locked nucleotide. In some embodiments, the nucleotide comprises at least two or more of: a length of from 10 to 30 nucleotides; a sequence comprising from 30% to 60% GC content; and at least one locked nucleotide. In some embodiments, the nucleotide comprises a length of from 10 to 30 nucleotides; a sequence comprising from 30% to 60% GC content; and at least one locked nucleotide.

The ASO can be any contiguous stretch of nucleic acids. In some embodiments, the ASO can be any contiguous stretch of deoxyribonucleic acid (DNA), RNA, non-natural, artificial nucleic acid, modified nucleic acid or any combination thereof. The ASO can be a linear nucleotide. In some embodiments, the ASO is an oligonucleotide. In some embodiments, the ASO is a single stranded polynucleotide. In some embodiments, the polynucleotide is pseudo-double stranded (e.g., a portion of the single stranded polynucleotide self-hybridizes).

In some embodiments, the ASO is an unmodified nucleotide. In some embodiments, the ASO is a modified nucleotide. As used herein, the term “modified nucleotide” refers to a nucleotide with at least one modification to the sugar, the nucleobase, or the internucleoside linkage.

In some embodiments, the ASOs described herein is single stranded, chemically modified and synthetically produced. In some embodiments, the ASOs described herein may be modified to include high affinity RNA binders (e.g., locked nucleic acids (LNAs)) as well as chemical modifications. In some embodiments, the ASO comprises one or more residues that are modified to increase nuclease resistance, and/or to increase the affinity of the ASO for the target sequence. In some embodiments, the ASO comprises a nucleotide analogue. In some embodiments, the ASO may be expressed inside a target cell, such as a neuronal cell, from a nucleic acid sequence, such as delivered by a viral (e.g. lentiviral, AAV, or adenoviral) or non-viral vector.

In some embodiments, the ASOs described herein is at least partially complementary to a target ribonucleotide. In some embodiments, the ASOs are complementary nucleic acid sequences designed to hybridize under stringent conditions to an RNA. In some embodiments, the oligonucleotides are chosen that are sufficiently complementary to the target, i.e., that hybridize sufficiently well and with sufficient specificity, to confer the desired effect.

In some embodiments, the ASO targets a Rluc RNA. In some embodiments, Rluc targeting ASO comprises a sequence having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% identity to SEQ ID NO: 2 or 3. In some embodiments, the ASO comprises SEQ ID NO: 2 or 3 optionally with one or more substitutions. In some embodiments, the ASO consists of SEQ ID NO: 2 or 3 optionally with one or more substitutions. In some embodiments, the ASO is selected from the group consisting of ASO2 and ASO3 shown in Table 1A or Table 1B below.

TABLE 1A
Sequences of exemplary ASOs targeting Renilla Luciferase (Rluc) RNA

| | | Human Genome |
|---|---|---|
| ASO Name | Sequence (5′-3′) | Coordinate (hg38) |
| ASO1 | AGAGGTGGCGTGGTAG | None |
| (non-targetting; control) | (SEQ ID NO: 1) | |
| ASO2 | TGTGTCAGAAGAATCAAGC | None |
| | (SEQ ID NO: 2) | |
| ASO3 | TTCTGCAGCTTAAGTTCGA | None |
| | (SEQ ID NO: 3) | |

In some embodiments, the ASO described herein may be chemically modified. In some embodiments, one or more nucleotides of the ASO described herein may be chemically modified with internal 2′-MethoxyEthoxy (i2MOEr) and/or 3′-Hydroxy-2′-MethoxyEthoxy (32MOEr), for example, resulting in those shown in Table 1B below.

TABLE 1B
Chemical Modifications of ASOs targeting Renilla luciferase (Rluc)
and a non-targeting (Scramble) ASO

| ASO | |
|---|---|
| name | Chemical modifications to ASO |
| ASO1 | */i2MOErA/*/i2MOErG/*/i2MOErA/*/i2MOErG/*/i2MOErG/*/ |
| | i2MOErT/*/i2MOErG/*/i2MOErG/*/i2MOErC/*/i2MOErG/*/ |
| | i2MOErT/*/i2MOErG/*/i2MOErG/*/i2MOErT/*/i2MOErA/*/ |
| | 32MOErG/ |
| ASO2 | */i2MOErT/*/i2MOErG/*/i2MOErT/*/i2MOErG/*/i2MOErT/*/ |
| | i2MOErC/*/i2MOErA/*/i2MOErG/*/i2MOErA/*/i2MOErA/*/ |
| | i2MOErG/*/i2MOErA/*/i2MOErA/*/i2MOErT/*/i2MOErC/*/ |
| | i2MOErA/*/i2MOErA/*/i2MOErG/*/32MOErC/ |
| ASO3 | */i2MOErT/*/i2MOErT/*/i2MOErC/*/i2MOErT/*/i2MOErG/*/ |
| | i2MOErC/*/i2MOErA/*/i2MOErG/*/i2MOErC/*/i2MOErT/*/ |
| | i2MOErT/*/i2MOErA/*/i2MOErA/*/i2MOErG/*/i2MOErT/*/ |
| | i2MOErT/*/i2MOErC/*/i2MOErG/*/32MOErA/ |

Table 1A shows ASO sequences and their coordinates in the human genome. Table 1B shows exemplary chemistry modifications for each ASOs. Mod Code follows IDT Mod Code: +=LNA, *=Phosphorothioate linkage, “r” signifies ribonucleotide, i2MOErA=internal 2′-MethoxyEthoxy A, i2MOErC=internal 2′-MethoxyEthoxy MeC, 32MOErA=3′-Hydroxy-2′-MethoxyEthoxy A etc.

As used herein, the term “RLuc” or “Rluc” refers to Renilla luciferase or Renilla-luciferin 2-monooxygenase. Renilla luciferase enzyme/protein purified from sea pansy (Renilla reniformis) is a bioluminescent soft coral that displays blue-green bioluminescence upon mechanical stimulation. It is also widely distributed among coelenterates, fishes, squids, and shrimps. It has been cloned and sequenced and used as a marker of gene expression in bacteria, yeast, plant, and mammalian cells. The enzyme RL catalyzes coelenterazine oxidation leading to bioluminescence.

### ASO Modification

In some embodiments, the ASO comprises one or more locked nucleic acids (LNA). In some embodiments, the ASO comprises at least one locked nucleotide. In some embodiments, the ASO comprises at least two locked nucleotides. In some embodiments, the ASO comprises at least three locked nucleotides. In some embodiments, the ASO comprises at least four locked nucleotides. In some embodiments, the ASO comprises at least five locked nucleotides. In some embodiments, the ASO comprises at least six locked nucleotides. In some embodiments, the ASO comprises at least seven locked nucleotides. In some embodiments, the ASO comprises at least eight locked nucleotides. In some embodiments, the ASO comprises a 5′ locked terminal nucleotide. In some embodiments, the ASO comprises a 3′ locked terminal nucleotide. In some embodiments, the ASO comprises a 5′ and a 3′ locked terminal nucleotides. In some embodiments, the ASO comprises a locked nucleotide near the 5′ end. In some embodiments, the ASO comprises a locked nucleotide near the 3′ end. In some embodiments, the ASO comprises locked nucleotides near the 5′ and the 3′ ends. In some embodiments, the ASO comprises a 5′ locked terminal nucleotide, a locked nucleotide at the second position from the 5′ end, a locked nucleotide at the third position from the 5′ end, a locked nucleotide at the fourth position from the 5′ end, a locked nucleotide at the fifth position from the 5′ end, or a combination thereof. In some embodiments, the ASO comprises a 3′ locked terminal nucleotide, a locked nucleotide at the second position from the 3′ end, a locked nucleotide at the third position from the 3′ end, a locked nucleotide at the fourth position from the 3′ end, a locked nucleotide at the fifth position from the 3′ end, or a combination thereof.

In some embodiments, the ASO can comprise one or more substitutions, insertions and/or additions, deletions, and covalent modifications with respect to reference sequences.

In some embodiments, the ASO as described herein includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27: 196-197).

In some embodiments, the ASO as described herein may include any useful modification, such as to the sugar, the nucleobase, or the internucleoside linkage (e.g., to a linking phosphate/to a phosphodiester linkage/to the phosphodiester backbone). In some embodiments, the ASO as described herein may include a modified nucleobase, a modified nucleoside, or a combination thereof.

In some embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In some embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone.

In some further embodiments, the ASO as described herein comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the ASO as described herein comprises at least one nucleoside selected from the group consisting of 5-azacytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the ASO as described herein comprises at least one nucleoside selected from the group consisting of 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, the nucleotides as described herein comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

Further nucleobases include those disclosed in Merigan et ah, U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

In some embodiments, modified nucleosides comprise double-headed nucleosides having two nucleobases. Such compounds are described in detail in Sorinas et al, J. Org. Chem, 2014 79: 8020-8030.

In some embodiments, the ASO as described herein comprises or consists of a modified oligonucleotide complementary to an target nucleic acid comprising one or more modified nucleobases. In some embodiments, the modified nucleobase is 5-methylcytosine. In some embodiments, each cytosine is a 5-methylcytosine.

In some embodiments, one or more atoms of a pyrimidine nucleobase in the ASO may be replaced or substituted with optionally substituted amino, optionally substituted thiol, optionally substituted alkyl (e.g., methyl or ethyl), or halo (e.g., chloro or fluoro). In some embodiments, modifications (e.g., one or more modifications) are present in each of the sugar and the internucleoside linkage. Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof. Additional modifications are described herein.

In some embodiments, the ASO as described herein includes at least one N(6)methyladenosine (m6A) modification. In some embodiments, the N(6)methyladenosine (m6A) modification can reduce immunogenicity of the nucleotide as described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

In some embodiments, chemical modifications to the nucleotide as described herein may enhance immune evasion. The ASO as described herein may be synthesized and/or modified by methods well established in the art, such as those described in “Current protocols in nucleic acid chemistry,” Beaucage, S. L. et al. (Eds.), John Wiley & Sons, Inc., New York, N.Y., USA, which is hereby incorporated herein by reference. Modifications include, for example, end modifications, e.g., 5′ end modifications (phosphorylation (mono-, di- and tri-), conjugation, inverted linkages, etc.), 3′ end modifications (conjugation, DNA nucleotides, inverted linkages, etc.), base modifications (e.g., replacement with stabilizing bases, destabilizing bases, or bases that base pair with an expanded repertoire of partners), removal of bases (abasic nucleotides), or conjugated bases. The modified nucleotide bases may also include 5-methylcytidine and pseudouridine. In some embodiments, base modifications may modulate expression, immune response, stability, subcellular localization, to name a few functional effects, of the nucleotide as described herein. In some embodiments, the modification includes a bi-orthogonal nucleotides, e.g., an unnatural base. See for example, Kimoto et al, Chem Commun (Camb), 2017, 53:12309, DOI: 10.1039/c7cc06661a, which is hereby incorporated by reference.

In some embodiments, sugar modifications (e.g., at the 2′ position or 4′ position) or replacement of the sugar of one or more nucleotides as described herein may, as well as backbone modifications, include modification or replacement of the phosphodiester linkages. Specific examples of the nucleotide as described herein include, but are not limited to the nucleotide as described herein including modified backbones or no natural internucleoside linkages such as internucleoside modifications, including modification or replacement of the phosphodiester linkages. The ASO having modified backbones include, among others, those that do not have a phosphorus atom in the backbone. For the purposes of this application, and as sometimes referenced in the art, modified nucleotides that do not have a phosphorus atom in their internucleoside backbone can also be considered to be oligonucleosides. In particular embodiments, the ASO will include nucleotides with a phosphorus atom in its internucleoside backbone.

In some embodiments, the ASO described herein may comprise one or more of (A) modified nucleosides and (B) Modified Internucleoside Linkages.

(A) Modified Nucleosides

Modified nucleosides comprise a modified sugar moiety, a modified nucleobase, or both a modified sugar moiety and a modified nucleobase.

1. Certain Modified Sugar Moieties

In certain embodiments, sugar moieties are non-bicyclic, modified furanosyl sugar moieties. In some embodiments, modified sugar moieties are bicyclic or tricyclic furanosyl sugar moieties. In some embodiments, modified sugar moieties are sugar surrogates. Such sugar surrogates may comprise one or more substitutions corresponding to those of other types of modified sugar moieties.

For example, in some embodiments, modified sugar moieties are non-bicyclic modified furanosyl sugar moieties comprising one or more acyclic substituent, including but not limited to substituents at the 2′, 3′, 4′, and/or 5′ positions. In some embodiments, the furanosyl sugar moiety is a ribosyl sugar moiety. In some embodiments, the furanosyl sugar moiety is a β-D-ribofuranosyl sugar moiety. In some embodiments, one or more acyclic substituent of non-bicyclic modified sugar moieties is branched.

Examples of 2′-substituent groups suitable for non-bicyclic modified sugar moieties include but are not limited to: 2′-F, 2′-OCH3 (“2′-OMe” or “2′-O-methyl”), and 2′-O(CH2)₂OCH3 (“2′-MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (N02), thiol, thioalkoxy, thioalkyl, halogen, alkyl, aryl, alkenyl and alkynyl. Examples of 3′-substituent groups include 3′-methyl (see Frier, et al., The ups and downs of nucleic acid duplex stability: structure-stability studies on chemically-modified DNA:RNA duplexes. Nucleic Acids Res., 25, 4429-4443, 1997.) Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-allyl, 5′-ethyl, 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836. 2′,4′-difluoro modified sugar moieties have been described in Martinez-Montero, et al., Rigid 2′, 4′-difluororibonucleosides: synthesis, conformational analysis, and incorporation into nascent RNA by HCV polymerase. J. Org. Chem., 2014, 79:5627-5635. Modified sugar moieties comprising a 2′-modification (OMe or F) and a 4′-modification (OMe or F) have also been described in Malek-Adamian, et al., J. Org. Chem, 2018, 83: 9839-9849.

In certain embodiments, a 2′-substituted nucleoside or non-bicyclic 2′-modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH2C(═O)—N(Rₘ)(Rₙ)), where each Rₘ and Rₐ is, independently, H, an amino protecting group, or substituted or unsubstituted C₁-C₁₀ alkyl.

In certain embodiments, a 2′-substituted nucleoside or non-bicyclic 2′-modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or non-bicyclic 2′-modified nucleoside comprises a sugar moiety comprising a non-bridging 2′-substituent group selected from: F, OCH3, and OCH2CH2OCH3.

In certain embodiments, the 4′ 0 of 2′-deoxyribose can be substituted with a S to generate 4′-thio DNA (see Takahashi, et al., Nucleic Acids Research 2009, 37: 1353-1362). This modification can be combined with other modifications detailed herein. In certain such embodiments, the sugar moiety is further modified at the 2′ position. In certain embodiments the sugar moiety comprises a 2′-fluoro. A thymidine with this sugar moiety has been described in Watts, et al., J. Org. Chem. 2006, 71(3): 921-925 (4′-S-fluoro5-methylarauridine or FAMU).

Certain modified sugar moieties comprise a bridging sugar substituent that forms a second ring resulting in a bicyclic sugar moiety. For example, in some embodiments, the bicyclic sugar moiety comprises a bridge between the 4′ and the 2′ furanose ring atoms. In some embodiments, the furanose ring is a ribose ring. Examples of sugar moieties comprising such 4′ to 2′ bridging sugar substituents include but are not limited to bicyclic sugars comprising: 4′-CH2-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al, J. Org. Chem., 2009, 74, 118-134), and 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b}, is. independently, H, a protecting group, or C₁-C₁₂ alkyl (see, e.g. Imanishi et al., U.S. Pat. No. 7,427,672), 4′-C(═O)—N(CH₃)₂-2′, 4′-C(═O)—N(R)₂-2′, 4′-C(═S)—N(R)₂-2′ and analogs thereof (see, e.g., Obika et al., WO2011052436A1, Yusuke, WO2017018360A1).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—. —C(Rₐ)═N—, —C(═NRₐ)—, —C(═O)—, —C(═S)—, —O—, —Si(Rₐ)₂—, —S(═O)ₓ—, and —N(Rₐ)—; wherein: x is 0, 1, or 2; n is 1, 2, 3, or 4; each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂. SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al, Nucleic Acids Research, 1997, 25(22), 4429-4443, Albaek et al, J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2017, 129, 8362-8379; Elayadi et al.; Christiansen, et al., J. Am. Chem. Soc. 1998, 120, 5458-5463; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al, U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In some embodiments, bicyclic sugar moieties and nucleosides incorporating such bicyclic sugar moieties are further defined by isomeric configuration. For example, an UNA nucleoside (described herein) may be in the α-U configuration or in the R-D configuration as follows:

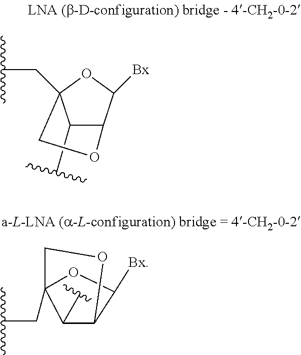

α-U-methyleneoxy (4′-CH₂—O-2′) or α-U-UNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., FNA) are identified in exemplified embodiments herein, they are in the β-D configuration, unless otherwise specified.

In some embodiments, modified sugar moieties comprise one or more non-bridging sugar substituent and one or more bridging sugar substituent (e.g., 5′-substituted and 4′-2′ bridged sugars).

Nucleosides comprising modified furanosyl sugar moieties and modified furanosyl sugar moieties may be referred to by the position(s) of the substitution(s) on the sugar moiety of the nucleoside. The term “modified” following a position of the furanosyl ring, such as “2′-modified”, indicates that the sugar moiety comprises the indicated modification at the 2′ position and may comprise additional modifications and/or substituents. A 4′-2′ bridged sugar moiety is 2′-modified and 4′-modified, or, alternatively, “2′, 4′-modified”. The term “substituted” following a position of the furanosyl ring, such as “2′-substituted” or “2′-4′-substituted”, indicates that is the only position(s) having a substituent other than those found in unmodified sugar moieties in oligonucleotides. Accordingly, the following sugar moieties are represented by the following formulas.

In the context of a nucleoside and/or an oligonucleotide, a non-bicyclic, modified furanosyl sugar moiety is represented by formula I.

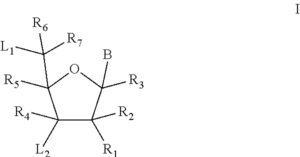

wherein B is a nucleobase; and L₁ and L₂ are each, independently, an internucleoside linkage, a terminal group, a conjugate group, or a hydroxyl group. Among the R groups, at least one of R₃₋₇ is not H and/or at least one of R₁ and R₂ is not H or OH. In a 2′-modified furanosyl sugar moiety, at least one of R₁ and R₂ is not H or OH and each of R₃₋₇ is independently selected from H or a substituent other than H. In a 4′-modified furanosyl sugar moiety, R₅ is not H and each of R_{1-4, 6, 7} are independently selected from H and a substituent other than H; and so on for each position of the furanosyl ring. The stereochemistry is not defined unless otherwise noted.

In the context of a nucleoside and/or an oligonucleotide, a non-bicyclic, modified, substituted fuamosyl sugar moiety is represented by formula I, wherein B is a nucleobase; and L₁ and L₂ are each, independently, an internucleoside linkage, a terminal group, a conjugate group, or a hydroxyl group. Among the R groups, either one (and no more than one) of R₃₋₇ is a substituent other than H or one of R₁ or R₂ is a substituent other than H or OH. The stereochemistry is not defined unless otherwise noted. Examples of non-bicyclic, modified, substituted furanosyl sugar moieties include 2′-substituted ribosyl, 4′-substituted ribosyl, and 5′-substituted ribosyl sugar moieties, as well as substituted 2′-deoxyfuranosyl sugar moieties, such as 4′-substituted 2′-deoxyribosyl and 5′-substituted 2′-deoxyribosyl sugar moieties.

In the context of a nucleoside and/or an oligonucleotide, a 2′-substituted ribosyl sugar moiety is represented by formula II:

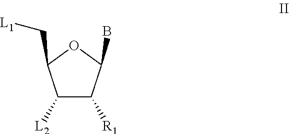

wherein B is a nucleobase; and L₁ and L₂ are each, independently, an internucleoside linkage, a terminal group, a conjugate group, or a hydroxyl group. R₁ is a substituent other than H or OH. The stereochemistry is defined as shown.

In the context of a nucleoside and/or an oligonucleotide, a 4′-substituted ribosyl sugar moiety is represented by formula III:

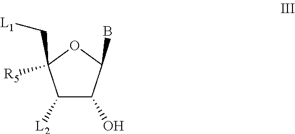

wherein B is a nucleobase; and L₁ and L₂ are each, independently, an internucleoside linkage, a terminal group, a conjugate group, or a hydroxyl group. R₅ is a substituent other than H. The stereochemistry is defined as shown.

In the context of a nucleoside and/or an oligonucleotide, a 5′-substituted ribosyl sugar moiety is represented by formula IV:

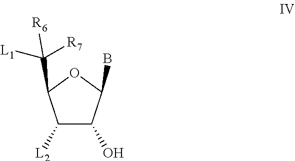

wherein B is a nucleobase; and L₁ and L₂ are each, independently, an internucleoside linkage, a terminal group, a conjugate group, or a hydroxyl group. R₆ or R₇ is a substituent other than H. The stereochemistry is defined as shown.

In the context of a nucleoside and/or an oligonucleotide, a 2′-deoxyfuranosyl sugar moiety is represented by formula V:

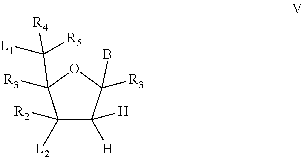

wherein B is a nucleobase; and L₁ and L₂ are each, independently, an internucleoside linkage, a terminal group, a conjugate group, or a hydroxyl group. Each of R₁-5 are independently selected from H and a non-H substituent. If all of R₁-5 are each H, the sugar moiety is an unsubstituted 2′-deoxyfuranosyl sugar moiety The stereochemistry is not defined unless otherwise noted.

In the context of a nucleoside and/or an oligonucleotide, a 4′-substituted 2′-deoxyribosyl sugar moiety is represented by formula VI:

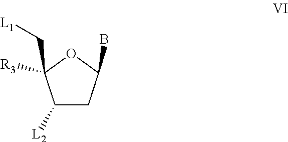

wherein B is a nucleobase; and L₁ and L₂ are each, independently, an internucleoside linkage, a terminal group, a conjugate group, or a hydroxyl group. R₃ is a substituent other than H. The stereochemistry is defined as shown.

In the context of a nucleoside and/or an oligonucleotide, a 5′-substituted 2′-deoxyribosyl sugar moiety is represented by formula VII:

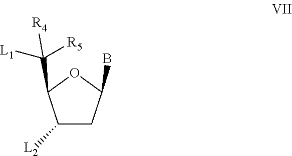

wherein B is a nucleobase; and L₁ and L₂ are each, independently, an internucleoside linkage, a terminal group, a conjugate group, or a hydroxyl group. R₄ or R₅ is a substituent other than H. The stereochemistry is defined as shown.

Unsubstituted 2′-deoxyfuranosyl sugar moieties may be unmodified (β-D-2′-deoxyribosyl) or modified. Examples of modified, unsubstituted 2′-deoxyfuranosyl sugar moieties include β-E-2′-deoxyribosyl, α-L-2′-deoxyribosyl, α-D-2′-deoxyribosyl, and β-D-xylosyl sugar moieties. For example, in the context of a nucleoside and/or an oligonucleotide, a β-L-2′-deoxyribosyl sugar moiety is represented by formula VIII:

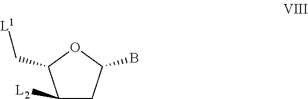

wherein B is a nucleobase; and L₁ and L₂ are each, independently, an internucleoside linkage, a terminal group, a conjugate group, or a hydroxyl group. The stereochemistry is defined as shown. Synthesis of α-L-ribosyl nucleotides and β-D-xylosyl nucleotides has been described by Gaubert, et al., Tetrahedron 2006, 62: 2278-2294. Additional isomers of DNA and RNA nucleosides are described by Vester, et al., “Chemically modified oligonucleotides with efficient RNase H response,” Bioorg. Med. Chem. Letters, 2008, 18: 2296-2300.

In some embodiments, modified sugar moieties are sugar surrogates. In some embodiments, the oxygen atom of the sugar moiety is replaced, e.g., with a sulfur, carbon or nitrogen atom. In some embodiments, such modified sugar moieties also comprise bridging and/or non-bridging substituents as described herein. For example, certain sugar surrogates comprise a 4′-sulfur atom and a substitution at the 2′-position (see, e.g., Bhat et al, U.S. Pat. No. 7,875,733 and Bhat et al, U.S. Pat. No. 7,939,677) and/or the 5′ position. In some embodiments, sugar surrogates comprise rings having other than 5 atoms. For example, in some embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), altritol nucleic acid (“ANA”), mannitol nucleic acid (“MNA”) (see. e.g., Leumann, C J. Bioorg. &Med. Chem. 2002, 10, 841-854), fluoro HNA (“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), F-CeNA, and 3′-ara-HNA, having the formulas below, where L₁ and L₂ are each, independently, an internucleoside linkage linking the modified THP nucleoside to the remainder of an oligonucleotide or one of L₁ and L₂ is an internucleoside linkage linking the modified THP nucleoside to the remainder of an oligonucleotide and the other of L₁ and L₂ is H, a hydroxyl protecting group, a linked conjugate group, or a 5′ or 3′-terminal group.

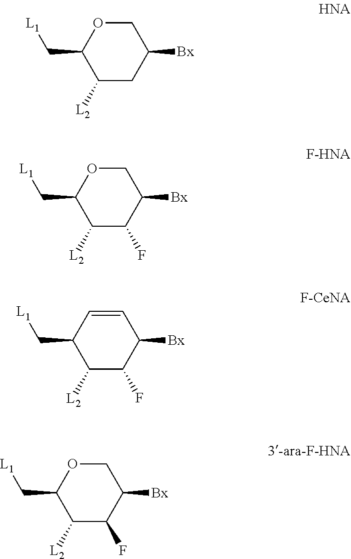

Additional sugar surrogates comprise THP compounds having the formula:

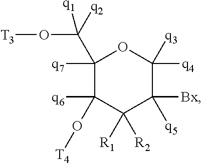

wherein, independently, for each of said modified THP nucleoside, Bx is a nucleobase moiety; T₃ and T₄ are each, independently, an internucleoside linkage linking the modified THP nucleoside to the remainder of an oligonucleotide or one of T₃ and T₄ is an internucleoside linkage linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having no heteroatoms. For example, nucleosides comprising bicyclo [3.1.0]-hexane have been described (see, e.g., Marquez, et al., J. Med. Chem. 1996, 39:3739-3749).

In some embodiments, sugar surrogates comprise rings having no heteroatoms. In some embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate comprising the following structure:

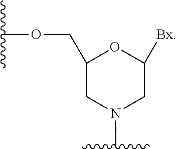

In some embodiments, morpholinos may be modified, for example by adding or altering various substituent groups from the above morpholino structure. Such sugar surrogates are referred to herein as “modified morpholinos.” In certain embodiments, morpholino residues replace a full nucleotide, including the internucleoside linkage, and have the structures shown below, wherein Bx is a heterocyclic base moiety.

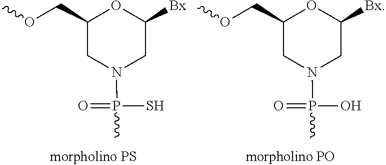

In some embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), glycol nucleic acid (“GNA,” see Schlegel, et al., J. Am. Chem. Soc. 2017, 139:8537-8546) and nucleosides and oligonucleotides described in Manoharan et al., WO2011/133876.

Many other bicyclic and tricyclic sugar and sugar surrogate ring systems are known in the art that can be used in modified nucleosides. Certain such ring systems are described in Hanessian, et al., J. Org. Chem., 2013, 78: 9051-9063 and include bcDNA and tcDNA. Modifications to bcDNA and tcDNA, such as 6′-fluoro, have also been described (Dogovic and Ueumann, J. Org. Chem., 2014, 79: 1271-1279).

In some embodiments, modified nucleosides are DNA or RNA mimics. “DNA mimic” or “RNA mimic” means a nucleoside other than a DNA nucleoside or an RNA nucleoside wherein the nucleobase is directly linked to a carbon atom of a ring bound to a second carbon atom within the ring, wherein the second carbon atom comprises a bond to at least one hydrogen atom, wherein the nucleobase and at least one hydrogen atom are trans to one another relative to the bond between the two carbon atoms.

In certain embodiments, a DNA mimic comprises a structure represented by the formula below:

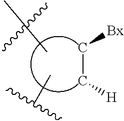

wherein Bx represents a heterocyclic base moiety.

In certain embodiments, a DNA mimic comprises a structure represented by one of the formulas below:

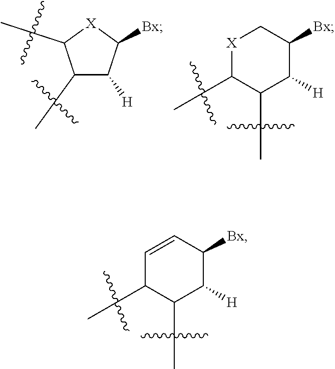

wherein X is O or S and Bx represents a heterocyclic base moiety.

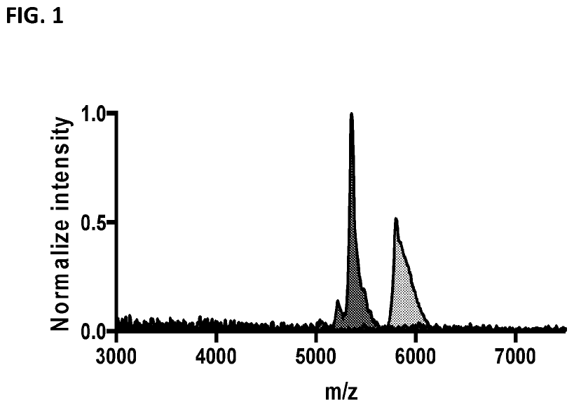

In certain embodiments, a DNA mimic is a sugar surrogate. In certain embodiments, a DNA mimic is a cycohexenyl or hexitol nucleic acid. In certain embodiments, a DNA mimic is described in FIG. 1 of Vester, et al., “Chemically modified oligonucleotides with efficient RNase H response,” Bioorg. Med. Chem. Letters, 2008, 18: 2296-2300, incorporated by reference herein. In certain embodiments, a DNA mimic nucleoside has a formula selected from:

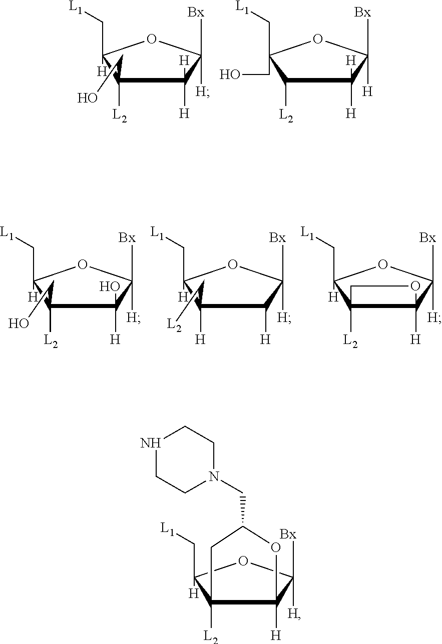

wherein Bx is a heterocyclic base moiety, and L₁ and L₂ are each, independently, an internucleoside linkage linking the modified THP nucleoside to the remainder of an oligonucleotide or one of L₁ and L₂ is an internucleoside linkage linking the modified nucleoside to the remainder of an oligonucleotide and the other of L₁ and L₂ is H, a hydroxyl protecting group, a linked conjugate group, or a 5′ or 3′-terminal group. In certain embodiments, a DNA mimic is α,β-constrained nucleic acid (CAN), 2′,4′-carbocyclic-LNA, or 2′, 4′-carbocyclic-ENA. In certain embodiments, a DNA mimic has a sugar moiety selected from among: 4′-C-hydroxymethyl-2′-deoxyribosyl, 3′-C-hydroxymethyl-2′-deoxyribosyl, 3′-C-hydroxymethyl-arabinosyl, 3′-C-2′-O-arabinosyl, 3′-C-methylene-extended-xyolosyl, 3′-C-2′-O-piperazino-arabinosyl. In certain embodiments, a DNA mimic has a sugar moiety selected from among: 2′-methylribosyl, 2′-S-methylribosyl, 2′-aminoribosyl, 2′-NH(CH₂)-ribosyl, 2′-NH(CH₂)₂-ribosyl, 2′-CH₂—F-ribosyl, 2′-CHF2-ribosyl, 2′-CF3-ribosyl, 2′=CF2 ribosyl, 2′-ethylribosyl, 2′-alkenylribosyl, 2′-alkynylribosyl, 2′-O-4′-C-methyleneribosyl, 2′-cyanoarabinosyl, 2′-chloroarabinosyl, 2′-fluoroarabinosyl, 2′-bromoarabinosyl, 2′-azidoarabinosyl, 2′-methoxyarabinosyl, and 2′-arabinosyl. In certain embodiments, a DNA mimic has a sugar moiety selected from 4′-methyl-modified deoxyfuranosyl, 4′-F-deoxyfuranosyl, 4′-OMe-deoxyfuranosyl. In certain embodiments, a DNA mimic has a sugar moiety selected from among: 5′-methyl-2′-p-D-deoxyribosyl, 5′-ethyl-2′-β-D-deoxyribosyl, 5′-allyl-2′-p-D-deoxyribosyl, 2-fluoro-β-D-arabinofuranosyl. In certain embodiments, DNA mimics are listed on page 32-33 of PCT/US00/267929 as B-form nucleotides, incorporated by reference herein in its entirety.

2. Modified Nucleobases

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443. In certain embodiments, modified nucleosides comprise double-headed nucleosides having two nucleobases. Such compounds are described in detail in Sorinas et al., J. Org. Chem, 2014 79: 8020-8030.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, compounds comprise or consist of a modified oligonucleotide complementary to an target nucleic acid comprising one or more modified nucleobases. In certain embodiments, the modified nucleobase is 5-methylcytosine. In certain embodiments, each cytosine is a 5-methylcytosine.

The backbones of the modified nucleotide as described herein may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the ASO may be negatively or positively charged.

(B) Modified Internucleoside Linkages

In certain embodiments, the modified nucleotides, which may be incorporated into the ASO, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates). The a-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked to the nucleotide as described herein is expected to reduce the innate immune response through weaker binding/activation of cellular innate immune molecules. For example, in some embodiments, a modified nucleoside includes an alpha-thio-nucleoside (e.g., 5′-O-(1-thiophosphate)-adenosine, 5′-O-(1-thiophosphate)-cytidine (a-thio-cytidine), 5′-O-(1-thiophosphate)-guanosine, 5′-O-(1-thiophosphate)-uridine, or 5′-0-(1-thiophosphate)-pseudouridine).

Other internucleoside linkages that may be employed according to the present disclosure, include internucleoside linkages which do not contain a phosphorous atom.

In some embodiments, the ASO having one or more modified internucleoside linkages are selected over compounds having only phosphodiester internucleoside linkages because of desirable properties such as, for example, enhanced cellular uptake, enhanced affinity for target nucleic acids, and increased stability in the presence of nucleases.

In some embodiments, compounds comprise or consist of a modified oligonucleotide complementary to a target nucleic acid comprising one or more modified internucleoside linkages. In some embodiments, the modified internucleoside linkages are phosphorothioate linkages. In some embodiments, each internucleoside linkage of an antisense compound is a phosphorothioate internucleoside linkage.

In some embodiments, nucleosides of modified oligonucleotides may be linked together using any internucleoside linkage. The two main classes of internucleoside linkages are defined by the presence or absence of a phosphorous atom. Representative phosphorus-containing internucleoside linkages include unmodified phosphodiester internucleoside linkages, modified phosphotriesters such as THP phosphotriester and isopropyl phosphotriester, phosphonates such as methylphosphonate, isopropyl phosphonate, isobutyl phosphonate, and phosphonoacetate, phosphoramidates, phosphorothioate, and phosphorodithioate (“HS—P═S”). Representative non-phosphorus containing internucleoside linkages include, but are not limited to, methylenemethylimino (—CH₂—N(CH₃)—O—CH₂—), thiodiester, thionocarbamate (—O—C(═O)(NH)—S—); siloxane; (—O—SiH₂—O—); formacetal, thioacetamido (TANA), alt-thioformacetal, glycine amide, and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified nucleotides comprising internucleoside linkages having a chiral center can be prepared as populations of modified nucleotides comprising stereorandom internucleoside linkages, or as populations of modified nucleotides comprising phosphorothioate linkages in particular stereochemical configurations. In some embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. All phosphorothioate linkages described herein are stereorandom unless otherwise specified. Nonetheless, as is well understood by those of skill in the art, each individual phosphorothioate of each individual oligonucleotide molecule has a defined stereoconfiguration.

In some embodiments, populations of modified oligonucleotides are enriched for modified oligonucleotides comprising one or more particular phosphorothioate internucleoside linkages in a particular, independently selected stereochemical configuration. In some embodiments, the particular configuration of the particular phosphorothioate linkage is present in at least 65% of the molecules in the population. In some embodiments, the particular configuration of the particular phosphorothioate linkage is present in at least 70% of the molecules in the population. In some embodiments, the particular configuration of the particular phosphorothioate linkage is present in at least 80% of the molecules in the population. In some embodiments, the particular configuration of the particular phosphorothioate linkage is present in at least 90% of the molecules in the population. In some embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al, JACS 125, 8307 (2003), Wan et al. Nuc. Acid. Res. 42, 13456 (2014), and WO 2017/015555.

In some embodiments, a population of modified oligonucleotides is enriched for modified nucleotides having at least one indicated phosphorothioate in the (Sp) configuration. In some embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

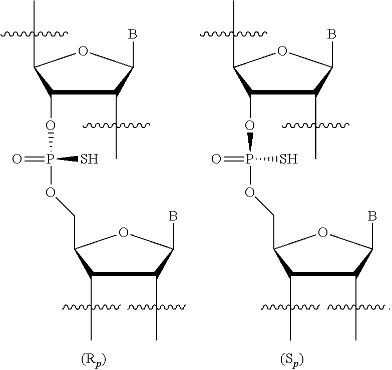

Unless otherwise indicated, chiral internucleoside linkages of modified oligonucleotides described herein can be stereorandom or in a particular stereochemical configuration.

In certain embodiments, nucleic acids can be linked 2′ to 5′ rather than the standard 3′ to 5′ linkage. Such a linkage is illustrated herein:

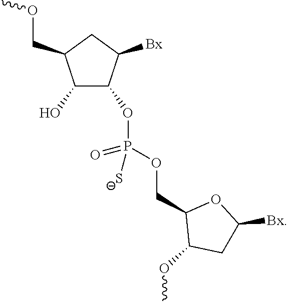

In the context of a nucleoside and/or an oligonucleotide, a non-bicyclic, 2′-linked modified furanosyl sugar moiety is represented by formula IX:

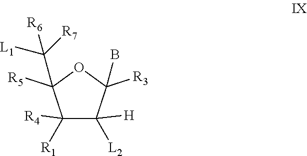

wherein B is a nucleobase; L₁ is an internucleoside linkage, a terminal group, a conjugate group, or a hydroxyl group and L₂ is an internucleoside linkage. The stereochemistry is not defined unless otherwise noted.

In certain embodiments, nucleosides can be linked by vinicinal 2′, 3′-phosphodiester bonds. In certain such embodiments, the nucleosides are threofuranosyl nucleosides (TNA; see Bala, et al., J Org. Chem. 2017, 82:5910-5916). A TNA linkage is shown herein:

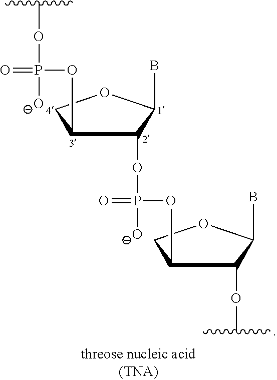

Neutral internucleoside linkages include, without limitation, phosphotriesters, phosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts. Additional modified linkages include α,β-D-CNA type linkages and related conformationally-constrained linkages, shown below. Synthesis of such molecules has been described previously (see Dupouy, et al., Angew. Chem. Int. Ed. Engl., 2014, 45: 3623-3627; Borsting, et al. Tetrahedron, 2004, 60: 10955-10966; Ostergaard, et al., ACS Chem. Biol. 2014, 9: 1975-1979; Dupouy, et al., Eur. J Org. Chem., 2008, 1285-1294; Martinez, et al., PLoS One, 2011, 6:e25510; Dupouy, et al., Eur. J Org. Chem., 2007, 5256-5264; Boissonnet, et al., New J. Chem., 2011, 35: 1528-1533).

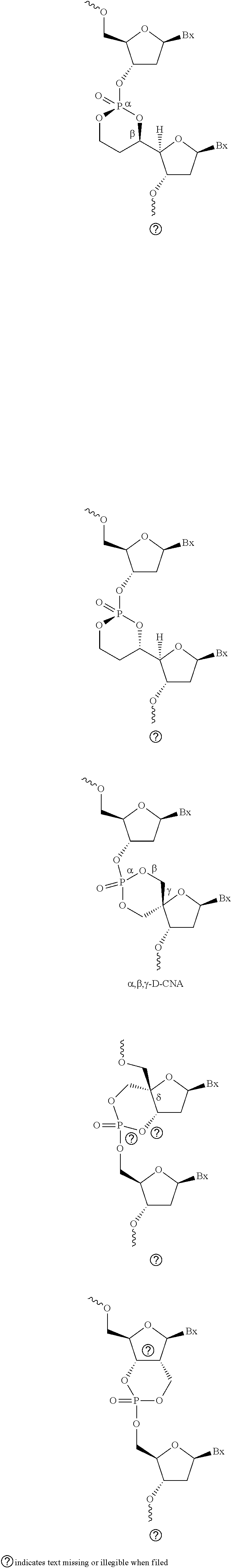

In some embodiments, the ASO may include one or more cytotoxic nucleosides. For example, cytotoxic nucleosides may be incorporated into the inhibitory nucleotide as described herein, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4(1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

The ASO may or may not be uniformly modified along the entire length of the molecule. For example, one or more or all types of nucleotide (e.g., naturally-occurring nucleotides, purine or pyrimidine, or any one or more or all of A, G, U, C, I, pU) may or may not be uniformly modified in the nucleotide as described herein, or in a given predetermined sequence region thereof. In some embodiments, the ASO includes a pseudouridine. In some embodiments, the ASO includes an inosine, which may aid in the immune system characterizing the ASO as endogenous versus viral RNAs. The incorporation of inosine may also mediate improved ASO stability/reduced degradation. See for example, Yu, Z. et al. (2015) RNA editing by ADARI marks dsRNA as “self”. Cell Res. 25, 1283-1284, which is incorporated by reference in its entirety.

In some embodiments, all nucleotides in the ASO (or in a given sequence region thereof) are modified. In some embodiments, the modification may include an m6A, which may augment expression; an inosine, which may attenuate an immune response; pseudouridine, which may increase RNA stability, an m5C, which may increase stability; and a 2,2,7-trimethylguanosine, which aids subcellular translocation (e.g., nuclear localization).

Different sugar modifications, nucleotide modifications, and/or internucleoside linkages (e.g., backbone structures) may exist at various positions in the nucleotide as described herein. One of ordinary skill in the art will appreciate that the nucleotide analogs or other modification(s) may be located at any position(s) of the nucleotide as described herein, such that the function of the nucleotide as described herein is not substantially decreased. A modification may also be a non-coding region modification. The nucleotide as described herein may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, modified nucleotides comprise one or more modified nucleoside comprising a modified sugar. In some embodiments, modified nucleotides comprise one or more modified nucleosides comprising a modified nucleobase. In some embodiments, modified nucleotides comprise one or more modified internucleoside linkage. In such embodiments, the modified, unmodified, and differently modified sugar moieties, nucleobases, and/or internucleoside linkages of a modified nucleotide define a pattern or motif. In some embodiments, the patterns or motifs of sugar moieties, nucleobases, and internucleoside linkages are each independent of one another. Thus, a modified nucleotide may be described by its sugar motif, nucleobase motif and/or internucleoside linkage motif (as used herein, nucleobase motif describes the modifications to the nucleobases independent of the sequence of nucleobases).

In some embodiments, the nucleotides comprise modified and/or unmodified nucleobases arranged along the oligonucleotide or region thereof in a defined pattern or motif. In some embodiments, each nucleobase is modified. In some embodiments, none of the nucleobases are modified. In some embodiments, each purine or each pyrimidine is modified. In some embodiments, each adenine is modified. In some embodiments, each guanine is modified. In some embodiments, each thymine is modified. In some embodiments, each uracil is modified. In some embodiments, each cytosine is modified. In some embodiments, some or all of the cytosine nucleobases in a modified nucleotide are 5-methylcytosines.

In some embodiments, modified nucleotides comprise a block of modified nucleobases. In some embodiments, the block is at the 3′-end of the nucleotide. In some embodiments, the block is within 3 nucleosides of the 3′-end of the nucleotide. In some embodiments, the block is at the 5′-end of the nucleotide. In some embodiments, the block is within 3 nucleosides of the 5′-end of the nucleotide.

In some embodiments, the nucleotides comprise modified and/or unmodified internucleoside linkages arranged along the nucleotide or region thereof in a defined pattern or motif. In some embodiments, each internucleoside linkage is a phosphodiester internucleoside linkage (P=0). In some embodiments, each internucleoside linkage of a modified nucleotide is a phosphorothioate internucleoside linkage (P═S). In some embodiments, each internucleoside linkage of a modified nucleotide is independently selected from a phosphorothioate internucleoside linkage and phosphodiester internucleoside linkage. In some embodiments, each phosphorothioate internucleoside linkage is independently selected from a stereorandom phosphorothioate, a (Sp) phosphorothioate, and a (Rp) phosphorothioate.

In some embodiments, the internucleoside linkages within the central region of a modified nucleotide are all modified. In some embodiments, some or all of the internucleoside linkages in the 5′-region and 3′-region are unmodified phosphate linkages. In some embodiments, the terminal internucleoside linkages are modified. In some embodiments, the internucleoside linkage motif comprises at least one phosphodiester internucleoside linkage in at least one of the 5′-region and the 3′-region, wherein the at least one phosphodiester linkage is not a terminal internucleoside linkage, and the remaining internucleoside linkages are phosphorothioate internucleoside linkages. In some embodiments, all of the phosphorothioate linkages are stereorandom. In some embodiments, all of the phosphorothioate linkages in the 5′-region and 3′-region are (Sp) phosphorothioates, and the central region comprises at least one Sp, Sp, Rp motif. In some embodiments, populations of modified oligonucleotides are enriched for modified oligonucleotides comprising such internucleoside linkage motifs.

In some embodiments, the nucleotides comprise a region having an alternating internucleoside linkage motif. In some embodiments, the nucleotides comprise a region of uniformly modified internucleoside linkages. In some embodiments, the internucleoside linkages are phosphorothioate internucleoside linkages. In some embodiments, all of the internucleoside linkages of the nucleotide are phosphorothioate internucleoside linkages. In some embodiments, each internucleoside linkage of the nucleotide is selected from phosphodiester or phosphate and phosphorothioate. In some embodiments, each internucleoside linkage of the nucleotide is selected from phosphodiester or phosphate and phosphorothioate and at least one internucleoside linkage is phosphorothioate.

In some embodiments, nucleotides comprise one or more methylphosphonate linkages. In some embodiments, modified nucleotides comprise a linkage motif comprising all phosphorothioate linkages except for one or two methylphosphonate linkages. In some embodiments, one methylphosphonate linkage is in the central region of an nucleotide.

In some embodiments, it is desirable to arrange the number of phosphorothioate internucleoside linkages and phosphodiester internucleoside linkages to maintain nuclease resistance. In some embodiments, it is desirable to arrange the number and position of phosphorothioate internucleoside linkages and the number and position of phosphodiester internucleoside linkages to maintain nuclease resistance. In some embodiments, the number of phosphorothioate internucleoside linkages may be decreased and the number of phosphodiester internucleoside linkages may be increased. In some embodiments, the number of phosphorothioate internucleoside linkages may be decreased and the number of phosphodiester internucleoside linkages may be increased while still maintaining nuclease resistance. In some embodiments, it is desirable to decrease the number of phosphorothioate internucleoside linkages while retaining nuclease resistance. In some embodiments, it is desirable to increase the number of phosphodiester internucleoside linkages while retaining nuclease resistance.

In some embodiments, the modifications as described herein (sugar, nucleobase, internucleoside linkage) are incorporated into a modified nucleotide. In some embodiments, modified nucleotides are characterized by their modifications, motifs, and overall lengths. In some embodiments, such parameters are each independent of one another. Thus, unless otherwise indicated, each internucleoside linkage of a modified nucleotide may be modified or unmodified and may or may not follow the modification pattern of the sugar moieties. Likewise, such modified nucleotides may comprise one or more modified nucleobase independent of the pattern of the sugar modifications. Furthermore, in certain instances, a modified nucleotide is described by an overall length or range and by lengths or length ranges of two or more regions (e.g., a region of nucleosides having specified sugar modifications), in such circumstances it may be possible to select numbers for each range that result in a nucleotide having an overall length falling outside the specified range. In such circumstances, both elements must be satisfied.

In some embodiments, the oligomeric compounds described herein comprise or consist of an oligonucleotide (modified or unmodified) and optionally one or more conjugate groups and/or terminal groups. Conjugate groups consist of one or more conjugate moiety and a conjugate linker that links the conjugate moiety to the oligonucleotide. Conjugate groups may be attached to either or both ends of an oligonucleotide and/or at any internal position. In some embodiments, conjugate groups are attached to the 2′-position of a nucleoside of a modified oligonucleotide. In some embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In some embodiments, conjugate groups are attached near the 3′-end of oligonucleotides. In some embodiments, conjugate groups (or terminal groups) are attached at the 5′-end of oligonucleotides. In some embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

In some embodiments, nucleotides are covalently attached to one or more conjugate groups. In some embodiments, conjugate groups modify one or more properties of the attached nucleotide, including but not limited to pharmacodynamics, pharmacokinetics, stability, binding, absorption, tissue distribution, cellular distribution, cellular uptake, charge and clearance. In some embodiments, conjugate groups impart a new property on the attached nucleotide, e.g., fluorophores or reporter groups that enable detection of the oligonucleotide.

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

Conjugate moieties are attached to the nucleotide through conjugate linkers. In certain oligomeric compounds, a conjugate linker is a single chemical bond (i.e. conjugate moiety is attached to an oligonucleotide via a conjugate linker through a single bond). In some embodiments, the conjugate linker comprises a chain structure, such as a hydrocarbyl chain, or an oligomer of repeating units such as ethylene glycol, nucleosides, or amino acid units.

In some embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In some embodiments, the conjugate linker comprises groups selected from alkyl and amide groups. In some embodiments, the conjugate linker comprises groups selected from alkyl and ether groups. In some embodiments, the conjugate linker comprises at least one phosphorus moiety. In some embodiments, the conjugate linker comprises at least one phosphate group. In some embodiments, the conjugate linker includes at least one neutral linking group.

In some embodiments, conjugate linkers, including the conjugate linkers described above, are bifunctional linking moieties, e.g., those known in the art to be useful for attaching conjugate groups to oligomeric compounds, such as the oligonucleotides provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In some embodiments, bifunctional linking moieties comprise one or more groups selected from amino, hydroxyl, carboxylic acid, thiol, alkyl, alkenyl, and alkynyl.

### First Domain Small Molecule

In some embodiments, the first domain of the bifunctional molecule as described herein, which specifically binds to a target RNA, is a small molecule. In some embodiments, the small molecule is selected from the group consisting of Table 2.

In some embodiments, the small molecule is an organic compound that is 1000 daltons or less. In some embodiments, the small molecule is an organic compound that is 900 daltons or less. In some embodiments, the small molecule is an organic compound that is 800 daltons or less. In some embodiments, the small molecule is an organic compound that is 700 daltons or less. In some embodiments, the small molecule is an organic compound that is 600 daltons or less. In some embodiments, the small molecule is an organic compound that is 500 daltons or less. In some embodiments, the small molecule is an organic compound that is 400 daltons or less.

As used herein, the term “small molecule” refers to a low molecular weight (<900 daltons) organic compound that may regulate a biological process. In some embodiments, small molecules bind nucleotide sequences or structures. In some embodiments, small molecules bind RNA sequences or structure. In some embodiments, small molecules bind modified nucleic acids. In some embodiments, small molecules bind endogenous nucleic acid sequences or structures. In some embodiments, small molecules bind exogenous nucleic acid sequences or structures. In some embodiments, small molecules bind artificial nucleic acid sequences. In some embodiments, small molecules bind biological macromolecules by covalent binding. In some embodiments, small molecules bind biological macromolecules by non-covalent binding. In some embodiments, small molecules bind biological macromolecules by irreversible binding. In some embodiments, small molecules bind biological macromolecules by reversible binding. In some embodiments, small molecules directly bind biological macromolecules. In some embodiments, small molecules indirectly bind biological macromolecules.

Routine methods can be used to design and identify small molecules that binds to the target sequence with sufficient specificity. In some embodiments, the methods include using bioinformatics methods known in the art to identify regions of secondary structure, e.g., one, two, or more stem-loop structures and pseudoknots, and selecting those regions to target with small molecules.

In some embodiments, the small molecule for purposes of the present methods may specifically bind the sequence to the target RNA or RNA structure and there is a sufficient degree of specificity to avoid non-specific binding of the sequence or structure to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency.

In general, the small molecule must retain specificity for their target, i.e., must not directly bind to, or directly significantly affect expression levels of, transcripts other than the intended target.

In some embodiments, the small molecules bind nucleotides. In some embodiments, the small molecules bind RNAs. In some embodiments, the small molecules bind modified nucleic acids. In some embodiments, the small molecules bind endogenous nucleic acid sequences or structures. In some embodiments, the small molecules bind exogenous nucleic acid sequences or structures. In some embodiments, the small molecules bind artificial nucleic acid sequences.

In some embodiments, the small molecules specifically bind to a target RNA by covalent bonds. In some embodiments, the small molecules specifically bind to a target RNA by non-covalent bonds. In some embodiments, the small molecules specifically bind to a target RNA sequence or structure by irreversible binding. In some embodiments, the small molecules specifically bind to a target RNA sequence or structure by reversible binding. In some embodiments, the small molecules specifically bind to a target RNA. In some embodiments, the small molecules specifically bind to a target RNA sequence or structure indirectly.

In some embodiments, the small molecules specifically bind to a nuclear RNA or a cytoplasmic RNA. In some embodiments, the small molecules specifically bind to an RNA involved in coding, decoding, regulation and expression of genes. In some embodiments, the small molecules specifically bind to an RNA that plays roles in protein synthesis, post-transcriptional modification, DNA replication, or any aspect of cellular physiology. In some embodiments, the small molecules specifically bind to a regulatory RNA. In some embodiments, the small molecules specifically bind to a non-coding RNA.

In some embodiments, the small molecules specifically bind to a specific region of the RNA sequence or structure. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity.

TABLE 2
Exemplary First Domain Small Molecules that Bind to RNA

| RNA-binding drug | Target RNA |
|---|---|
| Branaplam | SMN2 pre-mRNA |
| SMA-C5 | SMN2 pre-mRNA |
| ribocil | ribB riboswitch, mRNA |
| 2H—K4NMeS | DM1 CUG expansion mRNA |
| linezolid | 23S rRNA |
| sars-binding | sars (pseudoknot folds) |
| rpoH-mRNA binder | rpoH mRNA |
| aminoglycosides | pre-miRNA |
| yohimbine | IRES elements |
| “134” | U1 snRNA stem-loops |
| “16, 17, 18” | HIV TAR-RNA |
| mitoxanthrone, netilmicin | HIV TAR-RNA |
| “27, 28, 29” | Hep C IRES |
| thiamine, PT | tenA TPP riboswitch |
| oxazolidinones | Tbox riboswitch |
| 2,4 diaminopurine | purine riboswitches |
| RGB1, 2a, GQC-05 | 5′ utr IRES: NRAS, KRAS, BCL-X |
| 2-aminopurine | Adenine riboswitch |
| 2,4,5,6,-tetraminopyrimidine | Mutated G riboswitch |
| 2,4,6-triamino-1,3,5-triazine | Mutated G riboswitch |
| 2,4,6-triaminopyrimidine | Adenine riboswitch |
| 2-substituted aminopyridine | Ribosomal A-site (decoding center) |
| 2,6-diamonopurine | Adenine riboswitch |
| 2,7-quinolinediamine, N2,N2,4-trimethyl- | A-site |
| 3-quinolinecarboxamide | A-site |
| 4-pyridineacetamide, N-[2-(dimethylamino)-4- | A-site |
| methyl-7-quinolinyl] | |
| 5′-deoxy-5′-adenosylcobalamin (B12) | Riboswitch |
| ABT-773 | U2609 Escherichia coliribosome |
| Acetoperazine | HIV-1 TAR |
| Adenine | Adenine riboswitch |
| Amikacin | A-site |
| Anupam2b | T-box riboswitch |
| Anisomycin | Ribosome (PTC) |
| Apramycin | A-site |
| ATPA-18 | |
| Azithromycin | Ribosome (PTC) |
| B-13 and related | RNA hairpin loops |
| Benzimidazole13ibis | HCV IRES Domain II |
| Benzimidazole3ibis | HCV IRES domain II |
| Berenil | Poly(rA).2poly(rU) RNAtriplex/TAR |
| Biotin | Biotin aptamer |
| Blasticidin S | PTC |
| Carbomycin | 50S subunit |
| Chloramphenicol | 50S subunit |
| Chlorolissoclimide | Inhibitor of translocation |
| Chlorpromazine | HIV-1 TAR |
| Chlortetracycline | Small subunit |
| Clarithromycin | PTC |
| CMC1_dioxo-hexahydro-nitro-cyclopentaquinoxaline | HIV-1 TAR |
| CMC2_tetraaminoquinozaline | HIV-1 TAR |
| CMC3_Hoechst33258 | HIV-1 TAR |
| CMC3-1_Hoechst33258 | HIV-1 TAR/tRNA |
| CMC3-2_Hoechst33258 | HIV-1 TAR |
| CMC4_Hoechst33258 | Yeast tRNAphe |
| CMC6_diphenylfuran | HIV-1 RRE |
| CMC7_diphenylfuran | HIV-1 RRE |
| CMC8_diphenylfuran | HIV-1 RRE |
| Cycloheximide | |
| Dalfopristin | Large bacterial ribosomal subunit |
| DAPI | HIV-1 TAR |
| DB340 | HIV-1 RRE |
| Delfinidin | tRNA |
| Dichlorolissoclimide | Inhibit eukaryotic protein synthesis |
| Doxycycline | Small subunit |
| Erythromycin | PTC |
| Ethidium bromide | RNA/DNA heteroduplex, bulged RNA |
| Evernimicin | |
| FMN | Aptamer |
| Geneticin | Eubacterial A-site |
| Gentamicin C1A | Bacterial A-site |
| Glycine | Aptamer |
| Guanine | Guanine riboswitch |
| Hygromycin B | Small bacterial subunit |
| Hypoxanthine | Guanine riboswitch |
| Kanamycin A | Bacterial ribosomal A-site |
| Kanamycin B | A-site |
| Kasugamycin | Bacterial 70S ribosome |
| Linezolid | Bacterial ribosome |
| Lividomycin A | Bacterial ribosomal A-site |
| Malachite green | Aptamer |
| Methidiumpropyl | Bulged RNA |
| Micrococcin | L11 binding domain 50S subunit |
| Minocycline | Small subunit |
| Narciclasine | Eukaryotic ribosomal RNA |
| Negamycin | 50S exit tunnel |
| Neomycin | A-site, others |
| nf2 | A-site |
| nf3 | A-site |
| Nosiheptide | L11 binding domain, large subunit |
| Pactamycin | 30S subunit |
| Parkedavis1 | Group 1 intron |
| Parkdavis2 | Group 1 intron |
| Parkedavis3 | Group I Intron |
| Paromamine | Human A-site |
| Paromomycin | A-site |
| Paromomycin II | A-site |
| Pleuromutilin | PTC |
| Pristinamycin IIA | PTC |
| Promazine | HIV-1 TAR |
| Protoporphyrin IX | tRNA/M1 RNA |
| Puromycin | 50S A-site |
| Quenosine | Riboswitch |
| Quinacridone | HIV-1 TAR |
| Quinupristin | PTC |
| Ralenova (mitoxantrone) | HIV-1 psi RNA/hvg RNA |
| Rbt203 | HIV-1 TAR RNA |
| Rbt417 | HIV-1 TAR |
| Rbt418 | HIV-1 TAR |
| Rbt428 | HIV-1 TAR |
| Rbt489 | HIV-1 TAR |
| Rbt550 | HIV-1 TAR |
| Retapamulin | E. coli and Staphylococcus |
| | aureusribosomes |
| Ribostamycin | A-site/HIV dimerization site |
| S-adenosyl methionine | Riboswitch |
| Sisomicin | HCV IRES IIId |
| Spectinomycin | Small subunit |
| Spiramycin A | Exit tunnel, 50S |
| Streptogramin B | 50S subunit |
| T4-MPYP | tRNA, M1 RNA |
| Telithromycin | Large subunit |
| Tetracycline | Small subunit |
| Theophylline | Aptamer |
| Thiamine pyrophosphate | Riboswitch |
| Thiethylperazine | HIV-1 TAR |
| Thiostrepton | L11 binding domain |
| Tiamulin | PTC |
| Tigecycline | Small subunit |
| TMAP | tRNA/M1 RNA |
| Tobramicin | A-site/aptamer |
| Trifluoperazine | HIV-1 TAR |
| Tylosin | Exit tunnel, 50S |
| Usnic acid | HIV-1 TAR |
| Valnemulin | PTC |
| Viomycin | Ribosome intersubunit bridge |
| Wm5 | HIV-1 TAR |
| Xanthinol | HIV-1 TAR |
| Yohimbine | HIV-1 TAR |
| DPFp12 | MALAT1 |
| Risdiplam, Branaplam | SMN2 |
| 2H—5/2H—5NMe | CGG repeats in FMR1 |
| DC11, 2H—4KNMe, Bisamidinium 9 | CUG repeats in DMPK |
| (R)-N-(isoquinolin-1-yl)-3-(4-methoxyphenyl)-N- | PCSK9/ribosomal RNA |
| (piperidin-3-yl)propanamide (R-IMPP) | |
| RGB-1 | NRAS 5′UTR (G-quadruplex) |
| 4,11-bis(2-aminoethylamino)anthra[2,3-b]furan-5,10- | KRAS 5′UTR (G-quadruplex) |
| dione | |
| synucleozid | a-Synuclein |
| GQC-05 | BCl-X splice site (G-quadruplex) |
| CK1-14 | TERRA (G-quadruplex) |
| Targaprimir-96 | Pri-miR 96 |
| Targapremir-210 | Pre-miR 210 |
| TGP-377 | Pre-miR 377 |
| Compound(s) disclosed in Costales et al, PNAS, | Pre-miR 21 |
| 2020 117(5) 2406-2411. | |
| dihydropyrimidine | Aptamer 21 |
| thiazole orange | Mango aptamer |

### Target RNA

In some embodiments, a target ribonucleotide that comprises the target ribonucleic acid sequence or structure is a nuclear RNA or a cytoplasmic RNA. In some embodiments, the nuclear RNA or the cytoplasmic RNA is a long noncoding RNA (QncRNA), pre-mRNA, mRNA, microRNA, enhancer RNA, transcribed RNA, nascent RNA, chromosome-enriched RNA, ribosomal RNA, membrane enriched RNA, or mitochondrial RNA. In some embodiments, the target ribonucleic acid region is an intron. In some embodiments, the target ribonucleic acid region is an exon. In some embodiments, the target ribonucleic acid region is an untranslated region. In some embodiments, the target ribonucleic acid is a region translated into proteins. In some embodiments, the target sequence is translated or untranslated region on an mRNA or pre-mRNA. In some embodiments, a subcellular localization of the target RNA molecule is selected from the group consisting of nucleus, Golgi, endoplasmic reticulum, vacuole, lysosome, and mitochondrion. In some embodiments, the target RNA sequence or structure is located in an intron, an exon, a 5′ UTR, or a 3′ UTR of the target RNA molecule.

In some embodiments, the target ribonucleotide is an RNA involved in coding, noncoding, regulation and expression of genes. In some embodiments, the target ribonucleotide is an RNA that plays roles in protein synthesis, post-transcriptional modification, or DNA replication of a gene. In some embodiments, the target ribonucleotide is a regulatory RNA. In some embodiments, the target ribonucleotide is a non-coding RNA. In some embodiments, a region of the target ribonucleotide that the ASO or the small molecule specifically bind is selected from the full-length RNA sequence of the target ribonucleotide including all introns and exons.

A region that binds to the ASO or the small molecule can be a region of a target ribonucleotide. The region of the target ribonucleotide can comprise various characteristics. The ASO or the small molecule can then bind to this region of the target ribonucleotide. In some embodiments, the region of the target ribonucleotide that the ASO or the small molecule specifically binds is selected based on the following criteria: (i) a SNP frequency; (ii) a length; (iii) the absence of contiguous cytosines; (iv) the absence of contiguous identical nucleotides; (v) GC content; (vi) a sequence unique to the target ribonucleotide compared to a human transcriptome; (vii) the incapability of protein binding; and (viii) a secondary structure score. In some embodiments, the region of the target ribonucleotide comprises at least two or more of the above criteria. In some embodiments, the region of the target ribonucleotide comprises at least three or more of the above criteria. In some embodiments, the region of the target ribonucleotide comprises at least four or more of the above criteria. In some embodiments, the region of the target ribonucleotide comprises at least five or more of the above criteria. In some embodiments, the region of the target ribonucleotide comprises at least six or more of the above criteria. In some embodiments, the region of the target ribonucleotide comprises at least seven or more of the above criteria. In some embodiments, the region of the target ribonucleotide comprises eight of the above criteria. As used herein, the term “transcriptome” refers to the set of all RNA molecules (transcripts) in a specific cell or a specific population of cells. In some embodiments, it refers to all RNAs. In some embodiments, it refers to only mRNA. In some embodiments, it includes the amount or concentration of each RNA molecule in addition to the molecular identities.

In some embodiments, the region of the target ribonucleotide that the ASO or the small molecule specifically binds has a SNP frequency of less than 5%. As used herein, the term “single-nucleotide polymorphism” or “SNP” refers to a substitution of a single nucleotide that occurs at a specific position in the genome, where each variation is present at a level of more than 1% in the population. In some embodiments, the SNP falls within coding sequences of genes, non-coding regions of genes, or in the intergenic regions. In some embodiments, the SNP in the coding region is a synonymous SNP or a nonsynonymous SNP, in which the synonymous SNP does not affect the protein sequence, while the nonsynonymous SNP changes the amino acid sequence of protein. In some embodiments, the nonsynonymous SNP is missense or nonsense. In some embodiments, the SNP that is not in protein-coding regions affects RNA translation. In some embodiments, the region of the target ribonucleotide that the ASO or the small molecule specifically binds has a SNP frequency of less than 4%. In some embodiments, the region of the target ribonucleotide that the ASO or the small molecule specifically binds has a SNP frequency of less than 3%. In some embodiments, the region of the target ribonucleotide that the ASO or the small molecule specifically binds has a SNP frequency of less than 2%. In some embodiments, the region of the target ribonucleotide that the ASO or the small molecule specifically binds has a SNP frequency of less than 1%. In some embodiments, the region of the target ribonucleotide that the ASO or the small molecule specifically binds has a SNP frequency of less than 0.9%. In some embodiments, the region of the target ribonucleotide that the ASO or the small molecule specifically binds has a SNP frequency of less than 0.8%. In some embodiments, the region of the target ribonucleotide that the ASO or the small molecule specifically binds has a SNP frequency of less than 0.7%. In some embodiments, the region of the target ribonucleotide that the ASO or the small molecule specifically binds has a SNP frequency of less than 0.6%.

In some embodiments, the region of the target ribonucleotide that the ASO specifically binds has a SNP frequency of less than 0.5%. In some embodiments, the region of the target ribonucleotide that the ASO specifically binds has a SNP frequency of less than 0.4%. In some embodiments the region of the target ribonucleotide that the ASO specifically binds has a SNP frequency of less than 0.3%. the region of the target ribonucleotide that the ASO specifically binds has a SNP frequency of less than 0.2%. In some embodiments, the region of the target ribonucleotide that the ASO specifically binds has a SNP frequency of less than 0.10%.

In some embodiments, the region of the target ribonucleotide that the ASO specifically binds has a sequence comprising from 30% to 70% GC content. In some embodiments, the region of the target ribonucleotide that the ASO specifically binds has a sequence comprising from 40% to 70% GC content. In some embodiments, the region of the target ribonucleotide that the ASO specifically binds has a sequence comprising from 30% to 60% GC content. In some embodiments, the region of the target ribonucleotide that the ASO specifically binds has a sequence comprising from 40% to 60% GC content.

In some embodiments, the region of the target ribonucleotide that the ASO specifically binds has the length of from 8 to 30 nucleotides. In some embodiments, the region of the target ribonucleotide that the ASO specifically binds has the length of from 9 to 30 nucleotides. In some embodiments, the region of the target ribonucleotide that the ASO specifically binds has the length of from 10 to 30 nucleotides. In some embodiments, the region of the target ribonucleotide that the ASO specifically binds has the length of from 11 to 30 nucleotides. In some embodiments, the region of the target ribonucleotide that the ASO specifically binds has the length of from 12 to 30 nucleotides. In some embodiments, the region of the target ribonucleotide that the ASO specifically binds has the length of from 13 to 30 nucleotides. In some embodiments, the region of the target ribonucleotide that the ASO specifically binds has the length of from 14 to 30 nucleotides. In some embodiments, the region of the target ribonucleotide that the ASO specifically binds has the length of from 15 to 30 nucleotides. In some embodiments, the region of the target ribonucleotide that the ASO specifically binds has the length of from 16 to 30 nucleotides. In some embodiments, the region of the target ribonucleotide that the ASO specifically binds has the length of from 17 to 30 nucleotides. In some embodiments, the region of the target ribonucleotide that the ASO specifically binds has the length of from 18 to 30 nucleotides. In some embodiments, the region of the target ribonucleotide that the ASO specifically binds has the length of from 19 to 30 nucleotides. In some embodiments, the region of the target ribonucleotide that the ASO specifically binds has the length of from 20 to 30 nucleotides.

In some embodiments, the region of the target ribonucleotide that the ASO specifically binds has the length of from 8 to 29 nucleotides. In some embodiments, the region of the target ribonucleotide that the ASO specifically binds has the length of from 9 to 29 nucleotides. In some embodiments, the region of the target ribonucleotide that the ASO specifically binds has the length of from 10 to 29 nucleotides. In some embodiments, the region of the target ribonucleotide that the ASO specifically binds has the length of from 11 to 29 nucleotides. In some embodiments, the region of the target ribonucleotide that the ASO specifically binds has the length of from 12 to 29 nucleotides. In some embodiments, the region of the target ribonucleotide that the ASO specifically binds has the length of from 13 to 29 nucleotides. In some embodiments, the region of the target ribonucleotide that the ASO specifically binds has the length of from 14 to 29 nucleotides. In some embodiments, the region of the target ribonucleotide that the ASO specifically binds has the length of from 15 to 29 nucleotides. In some embodiments, the region of the target ribonucleotide that the ASO specifically binds has the length of from 16 to 29 nucleotides. In some embodiments, the region of the target ribonucleotide that the ASO specifically binds has the length of from 17 to 29 nucleotides. In some embodiments, the region of the target ribonucleotide that the ASO specifically binds has the length of from 18 to 29 nucleotides. In some embodiments, the region of the target ribonucleotide that the ASO specifically binds has the length of from 19 to 29 nucleotides. In some embodiments, the region of the target ribonucleotide that the ASO specifically binds has the length of from 20 to 29 nucleotides.

In some embodiments, the region of the target ribonucleotide that the ASO specifically binds has the length of from 8 to 28 nucleotides. In some embodiments, the region of the target ribonucleotide that the ASO specifically binds has the length of from 8 to 27 nucleotides. In some embodiments, the region of the target ribonucleotide that the ASO specifically binds has the length of from 8 to 26 nucleotides. In some embodiments, the region of the target ribonucleotide that the ASO specifically binds has the length of from 8 to 25 nucleotides. In some embodiments, the region of the target ribonucleotide that the ASO specifically binds has the length of from 8 to 24 nucleotides. In some embodiments, the region of the target ribonucleotide that the ASO specifically binds has the length of from 8 to 23 nucleotides. In some embodiments, the region of the target ribonucleotide that the ASO specifically binds has the length of from 8 to 22 nucleotides. In some embodiments, the region of the target ribonucleotide that the ASO specifically binds has the length of from 8 to 21 nucleotides. In some embodiments, the region of the target ribonucleotide that the ASO specifically binds has the length of from 8 to 20 nucleotides.

In some embodiments, the region of the target ribonucleotide that the ASO specifically binds has the length of from 10 to 28 nucleotides. In some embodiments, the region of the target ribonucleotide that the ASO specifically binds has the length of from 11 to 28 nucleotides. In some embodiments, the region of the target ribonucleotide that the ASO specifically binds has the length of from 12 to 28 nucleotides. In some embodiments, the region of the target ribonucleotide that the ASO specifically binds has the length of from 13 to 28 nucleotides. In some embodiments, the region of the target ribonucleotide that the ASO specifically binds has the length of from 14 to 28 nucleotides. In some embodiments, the region of the target ribonucleotide that the ASO specifically binds has the length of from 15 to 28 nucleotides.

In some embodiments, the region of the target ribonucleotide that the ASO specifically binds has the length of from 12 to 27 nucleotides. In some embodiments, the region of the target ribonucleotide that the ASO specifically binds has the length of from 12 to 26 nucleotides. In some embodiments, the region of the target ribonucleotide that the ASO specifically binds has the length of from 12 to 25 nucleotides. In some embodiments, the region of the target ribonucleotide that the ASO specifically binds has the length of from 12 to 24 nucleotides. In some embodiments, the region of the target ribonucleotide that the ASO specifically binds has the length of from 12 to 23 nucleotides. In some embodiments, the region of the target ribonucleotide that the ASO specifically binds has the length of from 12 to 22 nucleotides. In some embodiments, the region of the target ribonucleotide that the ASO specifically binds has the length of from 12 to 21 nucleotides. In some embodiments, the region of the target ribonucleotide that the ASO specifically binds has the length of from 12 to 20 nucleotides.

In some embodiments, the region of the target ribonucleotide that the ASO or the small molecule specifically binds has a sequence unique to the target ribonucleotide compared to a human transcriptome. In some embodiments, the region of the target ribonucleotide that the ASO or the small molecule specifically binds has a sequence lacking at least three contiguous cytosines. In some embodiments, the region of the target ribonucleotide that the ASO or the small molecule specifically binds has a sequence lacking at least four contiguous identical nucleotides. In some embodiments, the region of the target ribonucleotide that the ASO or the small molecule specifically binds has a sequence lacking four contiguous identical nucleotides. In some embodiments, the region of the target ribonucleotide that the ASO or the small molecule specifically binds has a sequence lacking four contiguous identical guanines. In some embodiments, the region of the target ribonucleotide that the ASO or the small molecule specifically binds has a sequence lacking four contiguous identical adenines. In some embodiments, the region of the target ribonucleotide that the ASO or the small molecule specifically binds has a sequence lacking four contiguous identical uracils.

In some embodiments, the region of the target ribonucleotide that the ASO or the small molecule specifically binds to does or does not bind a protein. In some embodiments, the region of the target ribonucleotide that the ASO or the small molecule specifically binds to does or does not comprise a sequence motif or structure motif suitable for binding to an RNA-recognition motif, double-stranded RNA-binding motif, K-homology domain, or zinc fingers of an RNA-binding protein. As a non-limiting example, the region of the target ribonucleotide that the ASO or the small molecule specifically binds does or does not have the sequence motif or structure motif listed in Pan et al., BMC Genomics, 19, 511 (2018) and Dominguez et al., Molecular Cell 70, 854-867 (2018); the contents of each of which are herein incorporated by reference in its entirety. In some embodiments, the region of the target ribonucleotide that an ASO specifically binds does or does not comprise a protein binding site. Examples of the protein binding site includes, but are not limited to, a binding site to the protein such as ACIN1, AGO, APOBEC3F, APOBEC3G, ATXN2, AUH, BCCIP, CAPRIN1, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIP1L1, FKBP4, FMR1, FUS, FXR1, FXR2, GNL3, GTF2F1, HNRNPA1, HNRNPA2B1, HNRNPC, HNRNPK, HNRNPL, HNRNPM, HNRNPU, HNRNPUL1, IGF2BP1, IGF2BP2, IGF2BP3, ILF3, KHDRBS1, LARP7, LIN28A, LIN28B, m6A, MBNL2, METTL3, MOV10, MSI1, MSI2, NONO, NONO−, NOP58, NPM1, NUDT21, PCBP2, POLR2A, PRPF8, PTBP1, RBFOX2, RBM10, RBM22, RBM27, RBM47, RNPS1, SAFB2, SBDS, SF3A3, SF3B4, SIRT7, SLBP, SLTM, SMNDC1, SND1, SRRM4, SRSF1, SRSF3, SRSF7, SRSF9, TAF15, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, AKT1, and any other protein that binds RNA.

In some embodiments, the region of the target ribonucleotide that the small molecule specifically binds has a secondary structure. In some embodiments, the region of the target ribonucleotide that the ASO specifically binds has a limited secondary structure. In some embodiments, the region of the target ribonucleotide that the small molecule specifically binds has unique secondary structure. In some embodiments, the secondary structure of a region of the target ribonucleotide is predicted by an RNA structure prediction software, such as CentroidFold, CentroidHomfold, Context Fold, CONTRAfold, Crumple, CyloFold, GTFold, IPknot, KineFold, Mfold, pKiss, Pknots, PknotsRG, RNA123, RNAfold, RNAshapes, RNAstructure, SARNA-Predict, Sfold, Sliding Windows & Assembly, SPOT-RNA, SwiSpot, UNAFold, and vsfold/vs subopt.

In some embodiments, the region of the target ribonucleotide that the ASO or the small molecule specifically binds has at least two or more of (i) a SNP frequency of less than 5%; (ii) a length of from 8 to 30 nucleotides; (iii) a sequence lacking three contiguous cytosines; (iv) a sequence lacking four contiguous identical nucleotides; (v) a sequence comprising from 30% to 70% GC content; (vi) a sequence unique to the target ribonucleotide compared to a human transcriptome; and (vii) no protein binding. In some embodiments, the region of the target ribonucleotide that the ASO or the small molecule specifically binds has at least three or more of (i) a SNP frequency of less than 5%; (ii) a length of from 8 to 30 nucleotides; (iii) a sequence lacking three contiguous cytosines; (iv) a sequence lacking four contiguous identical nucleotides; (v) a sequence comprising from 30% to 70% GC content; (vi) a sequence unique to the target ribonucleotide compared to a human transcriptome; and (vii) no protein binding. In some embodiments, the region of the target ribonucleotide that the ASO or the small molecule specifically binds has at least four or more of (i) a SNP frequency of less than 5%; (ii) a length of from 8 to 30 nucleotides; (iii) a sequence lacking three contiguous cytosines; (iv) a sequence lacking four contiguous identical nucleotides; (v) a sequence comprising from 30% to 70% GC content; (vi) a sequence unique to the target ribonucleotide compared to a human transcriptome; and (vii) no protein binding. In some embodiments, the region of the target ribonucleotide that the ASO or the small molecule specifically binds has at least five or more of (i) a SNP frequency of less than 5%; (ii) a length of from 8 to 30 nucleotides; (iii) a sequence lacking three contiguous cytosines; (iv) a sequence lacking four contiguous identical nucleotides; (v) a sequence comprising from 30% to 70% GC content; (vi) a sequence unique to the target ribonucleotide compared to a human transcriptome; and (vii) no protein binding. In some embodiments, the region of the target ribonucleotide that the ASO or the small molecule specifically binds has at least six or more of (i) a SNP frequency of less than 5%; (ii) a length of from 8 to 30 nucleotides; (iii) a sequence lacking three contiguous cytosines; (iv) a sequence lacking four contiguous identical nucleotides; (v) a sequence comprising from 30% to 70% GC content; (vi) a sequence unique to the target ribonucleotide compared to a human transcriptome; and (vii) no protein binding. In some embodiments, the region of the target ribonucleotide that the ASO or the small molecule specifically binds has at least seven or more of (i) a SNP frequency of less than 5%; (ii) a length of from 8 to 30 nucleotides; (iii) a sequence lacking three contiguous cytosines; (iv) a sequence lacking four contiguous identical nucleotides; (v) a sequence comprising from 30% to 70% GC content; (vi) a sequence unique to the target ribonucleotide compared to a human transcriptome; and (vii) no protein binding. In some embodiments, the region of the target ribonucleotide that the ASO or the small molecule specifically binds has (i) a SNP frequency of less than 5%; (ii) a length of from 8 to 30 nucleotides; (iii) a sequence lacking three contiguous cytosines; (iv) a sequence lacking four contiguous identical nucleotides; (v) a sequence comprising from 30% to 70% GC content; (vi) a sequence unique to the target ribonucleotide compared to a human transcriptome; and (vii) no protein binding.

In some embodiments, the ASO or the small molecule can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al, J. Mol. Biol., 1990, 215, 403-410; Zhang and Madden, Genome Res., 1997, 7, 649-656), e.g., using the default parameters.

In some embodiments, the bifunctional molecules bind to the target RNA and recruit a target polypeptide or a target protein (e.g., effector) as described herein, by binding of the target polypeptide or protein to the second domain. Alternatively, in some embodiments, the ASOs or the small molecules increase translation of the ribonucleic acid sequence, by binding to the target RNA by way of a target polypeptide or protein being recruited to the target site by the interaction between the second domain (e.g., effector recruiter) of the bifunctional molecule and the target polypeptide or the target protein (e.g., effector).

In some embodiments, the target RNA is a non-coding RNA or a coding RNA. In some embodiments, the target RNA or a gene is a Rluc RNA.

### Second Domain

In some embodiments, the second domain of the bifunctional molecule as described herein, which specifically binds to a target protein (e.g., an effector), comprises a small molecule or an aptamer. In some embodiments, the second domain specifically binds to the target polypeptide or protein. In some embodiments, the second domain binds to an active site, an allosteric site or an inert site on the target protein. In some embodiments, the target polypeptide or protein is endogenous. In some embodiments, the target protein is an exogenously introduced protein or fusion protein. In some embodiments the target polypeptide is an exogenous. In some embodiments the target polypeptide is a fusion protein or recombinant protein.

### Second Domain Small Molecule

In some embodiments, the second domain is a small molecule.

Routine methods can be used to design small molecules that binds to the target protein with sufficient specificity. In some embodiments, the small molecule for purposes of the present methods may specifically bind the sequence to the target protein to elicit the desired effects, e.g., increasing translation of a ribonucleic acid sequence, and there is a sufficient degree of specificity to avoid non-specific binding of the sequence to non-target protein under conditions in which specific binding is desired, e.g., under physiological conditions in the case of in vivo assays or therapeutic treatment, and in the case of in vitro assays, under conditions in which the assays are performed under suitable conditions of stringency.

In some embodiments, the small molecules bind an effector. In some embodiments, the small molecules bind proteins or polypeptides. In some embodiments, the small molecules bind endogenous proteins or polypeptides. In some embodiments, the small molecules bind exogenous proteins or polypeptides. In some embodiments, the small molecules bind recombinant proteins or polypeptides. In some embodiments, the small molecules bind artificial proteins or polypeptides. In some embodiments, the small molecules bind fusion proteins or polypeptides. In some embodiments, the small molecules bind enzymes. In some embodiments, the small molecules bind scaffolding proteins. In some embodiments, the small molecules bind a regulatory protein. In some embodiments, the small molecules bind receptors. In some embodiments, the small molecules bind signaling proteins or peptides. In some embodiments, the small molecules bind translation factors. In some embodiments, the small molecules bind translational regulators or mediators. In some embodiments, the small molecules bind proteins that recruit translation factors, translational regulators or translational mediators.

In some embodiments, the small molecules specifically bind to a target protein by covalent bonds. In some embodiments, the small molecules specifically bind to a target protein by non-covalent bonds. In some embodiments, the small molecules specifically bind to a target protein by irreversible binding. In some embodiments, the small molecules specifically bind to a target protein by reversible binding. In some embodiments, the small molecules specifically bind to a target protein through interaction with the side chains of the target protein. In some embodiments, the small molecules specifically bind to a target protein through interaction with the N-terminus of the target protein. In some embodiments, the small molecules specifically bind to a target protein through interaction with the C-terminus of the target protein. In some embodiments, the small molecules specifically binds to an active site, an allosteric site, or an inert site on the target protein or polypeptide.

In some embodiments, the small molecules specifically bind to a specific region of the target protein sequence. For example, a specific functional region can be targeted, e.g., a region comprising a catalytic domain, a kinase domain, a protein-protein interaction domain, a protein-DNA interaction domain, a protein-RNA interaction domain, a regulatory domain, a signal domain, a nuclear localization domain, a nuclear export domain, a transmembrane domain, a glycosylation site, a modification site, or a phosphorylation site. Alternatively or in addition, highly conserved regions can be targeted, e.g., regions identified by aligning sequences from disparate species such as primate (e.g., human) and rodent (e.g., mouse) and looking for regions with high degrees of identity.

As used herein, the term “Ibrutinib” or “Imbruvica” refers to a small molecule drug that binds permanently to Bruton's tyrosine kinase (BTK), more specifically binds to the ATP-binding pocket of BTK protein that is important in B cells. In some embodiments, Ibrutinib is used to treat B cell cancers like mantle cell lymphoma, chronic lymphocytic leukemia, and Waldenström's macroglobulinemia. In some embodiments, the second domain small molecule comprises a derivative of Ibrutinib. In some embodiments, the second domain small molecule comprises a derivative of Ibrutinib, including Ibrutinib-MPEA.

In some embodiments, the second domain small molecule comprises biotin.

### Aptamer

In some embodiments, the second domain of the bifunctional molecule as described herein, which specifically binds to a target polypeptide or protein is an aptamer.

As used herein, the term “aptamer” refers to oligonucleotide or peptide molecules that bind to a specific target molecule. In some embodiments, the aptamers bind to a target protein.

Routine methods can be used to design and select aptamers that binds to the target protein with sufficient specificity. In some embodiments, the aptamer for purposes of the present methods bind to the target protein to recruit the protein (e.g., effector). Once recruited, the protein itself performs the desired effects or the protein recruits another protein or protein complex to perform the desired effects, e.g., translating a ribonucleic acid sequence, and there is a sufficient degree of specificity to avoid non-specific binding of the sequence to non-target protein under conditions in which specific binding is desired, e.g., under physiological conditions in the case of in vivo assays or therapeutic treatment, and in the case of in vitro assays, under conditions in which the assays are performed under suitable conditions of stringency.

In some embodiments, the aptamers bind proteins or polypeptides. In some embodiments, the aptamers bind endogenous proteins or polypeptides. In some embodiments, the aptamers bind exogenous proteins or polypeptides. In some embodiments, the aptamers bind recombinant proteins or polypeptides. In some embodiments, the aptamers bind artificial proteins or polypeptides. In some embodiments, the aptamers bind fusion proteins or polypeptides. In some embodiments, the aptamers bind enzymes. In some embodiments, the aptamers bind scaffolding proteins. In some embodiments, the aptamers bind a regulatory protein. In some embodiments, the aptamers bind receptors. In some embodiments, the aptamers bind signaling proteins or peptides. In some embodiments, the aptamers bind translation factors. In some embodiments, the aptamers bind translational regulators or mediators. In some embodiments, the aptamers bind proteins that recruit translation factors, translational regulators or translational mediators.

In some embodiments, the aptamers specifically bind to a target protein by covalent bonds. In some embodiments, the aptamers specifically bind to a target protein by non-covalent bonds. In some embodiments, the aptamers specifically bind to a target protein by irreversible binding. In some embodiments, the aptamers specifically bind to a target protein by reversible binding. In some embodiments, the aptamers specifically binds to an active site, an allosteric site, or an inert site on the target polypeptide of protein.

In some embodiments, the aptamers specifically bind to a specific region of the target protein sequence. For example, a specific functional region can be targeted, e.g., a region comprising a catalytic domain, a kinase domain, a protein-protein interaction domain, a protein-DNA interaction domain, a protein-RNA interaction domain, a regulatory domain, a signal domain, a nuclear localization domain, a nuclear export domain, a transmembrane domain, a glycosylation site, a modification site, or a phosphorylation site. Alternatively or in addition, highly conserved regions can be targeted, e.g., regions identified by aligning sequences from disparate species such as primate (e.g., human) and rodent (e.g., mouse) and looking for regions with high degrees of identity.

In some embodiments, the aptamers increase the activity or function of the protein, e.g., translating a ribonucleic acid sequence, by binding to the target protein after recruited to the target site by the interaction between the first domain of the bifunctional molecule as described herein. Alternatively, the aptamers bind to the target protein and recruit the bifunctional molecule as described herein, thereby allowing the first domain to specifically bind to an RNA sequence of a target RNA.

In some embodiments, the second domain comprises an aptamer that binds to BTK. In some embodiments, the second domain comprises an aptamer that inhibits to BTK.

### Certain Conjugated Compounds

A. Certain Conjugate Groups

In certain embodiments, the small molecules or oligonucleotides are covalently attached to one or more conjugate groups. In certain embodiments, conjugate groups modify one or more properties of the attached small molecule or oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached small molecule or oligonucleotide, e.g., fluorophores or reporter groups that enable detection of the small molecule or oligonucleotide.

Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. NY. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J Pharmacol. Exp. Ther., 1996, i, 923-937), a tocopherol group (Nishina et al., Molecular Therapy Nucleic Acids, 2015, 4, e220; doi: 10.1038/mtna.2014.72 and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to small molecules or oligonucleotides through conjugate linkers. In certain small molecules or oligomeric compounds, a conjugate linker is a single chemical bond (i.e. conjugate moiety is attached to an small molecule or oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to small molecules or oligomeric compounds, such as the oligonucleotides provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such a compound is more than 30. Alternatively, an oligomeric compound may comprise a modified oligonucleotide consisting of 8-30 nucleosides and no conjugate group. The total number of contiguous linked nucleosides in such a compound is no more than 30. Unless otherwise indicated conjugate linkers comprise no more than 10 linker-nucleosides. In certain embodiments, conjugate linkers comprise no more than 5 linker-nucleosides.

In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the small molecule or oligonucleotide. For example, in certain circumstances small molecule or oligomeric compounds comprising a particular conjugate moiety are better taken up by a particular cell type, but once the compound has been taken up, it is desirable that the conjugate group be cleaved to release the unconjugated small molecule or oligonucleotide. Thus, certain conjugate may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate or phosphodiester linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is a nucleoside comprising a 2′-deoxyfuranosyl that is attached to either the 3′ or 5′-terminal nucleoside of an oligonucleotide by a phosphodiester internucleoside linkage and covalently attached to the remainder of the conjugate linker or conjugate moiety by a phosphodiester or phosphorothioate linkage. In certain such embodiments, the cleavable moiety is a nucleoside comprising a 2′-p-D-deoxyribosyl sugar moiety. In certain such embodiments, the cleavable moiety is 2′-deoxyadenosine.

3. Certain Cell-Targeting Conjugate Moieties

In certain embodiments, a conjugate group comprises a cell-targeting conjugate moiety. In certain embodiments, a conjugate group has the general formula:

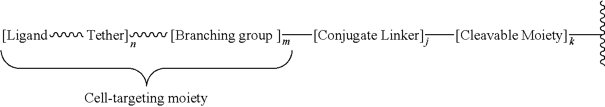

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian lung cell.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety). In certain such embodiments, each ligand is an amino sugar or athio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, oligomeric compounds or oligonucleotides described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vase Biol, 2006, 26, 169-175; van Rossenberg et al., Gene Ther, 2004, 11, 457-464; Sato et al., JAm Chem Soc, 2004, 126, 14013-14022; Lee et al., JOrg Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO 1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601: 7,262,177: 6,906,182: 6,620,916: 8,435,491: 8,404,862: 7,851,615: Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

### Target Polypeptide or Protein

In some embodiments, the target protein may be an effector. In other embodiments, the target proteins may be endogenous proteins or polypeptides. In some embodiments, the target proteins may be exogenous proteins or polypeptides. In some embodiments, the target proteins may be recombinant proteins or polypeptides. In some embodiments, the target proteins may be artificial proteins or polypeptides. In some embodiments, the target proteins may be fusion proteins or polypeptides. In some embodiments, the target proteins may be enzymes. In some embodiments, the target proteins may be scaffolding proteins. In some embodiments, the target proteins may be receptors. In some embodiments, the target proteins may be signaling proteins or peptides. In some embodiments, the target proteins may be translation factors. In some embodiments, the target proteins may be translational regulators or mediators.

In some embodiments, the activity or function of the target protein, e.g., translating a ribonucleic acid sequence, may be enhanced by binding to the second domain of the bifunctional molecule as provided herein. In some embodiments, the target protein recruits the bifunctional molecule as described herein by binding to the second domain of the bifunctional molecule as provided herein, thereby allowing the first domain to specifically bind to an RNA sequence of a target RNA. In some embodiments, the target protein further recruits additional functional domains or proteins.

In some embodiments, the target protein comprises a tyrosine kinase. In some embodiments, the target protein comprises a protein that mediates increasing RNA translation. In some embodiments, the target protein comprises a protein that increases RNA translation. In some embodiments, the target protein comprises a protein that increases RNA translation. In some embodiments, the target protein comprises a translational regulator.

In some embodiments, the target protein is a tyrosine kinase

In some embodiments, the target protein comprises BTK (Bruton's Tyrosine Kinase). In some embodiments, the target protein is Bruton's Tyrosine Kinase (BTK). In some embodiments, the target protein comprises a nuclear localization signal. In some embodiments, the target protein comprises a nuclear export signal.

As used herein, the term “BTK” or “Bruton's Tyrosine Kinase),” also known as tyrosine-protein kinase BTK, refers to a tyrosine kinase that is encoded by the BTK gene in humans. BTK plays a crucial role in B cell development. In some embodiments, BTK plays a crucial role in B cell development as it is required for transmitting signals from the pre-B cell receptor that forms after successful immunoglobulin heavy chain rearrangement. In some embodiments, BTK also has a role in mast cell activation through the high-affinity IgE receptor.

In some embodiments, the target protein comprises EIF4F. In some embodiments, the target protein is EIF4F. As used herein the term “EIF4F” refers to a complex of cellular polypeptides whose core is composed of a cap binding protein (eIF4E), a large scaffolding subunit (eIF4G), and an RNA helicase (eIF4A). Aberrant activity of this complex is observed in many cancers, leading to the selective synthesis of proteins involved in tumor growth and metastasis. The selective translation of cellular mRNAs controlled by this complex also contributes to resistance to cancer treatments, and downregulation of the EIF4F complex components can restore sensitivity to various cancer therapies.

In some embodiments, the target protein comprises an epitranscriptomic reader protein. In some embodiments, the target protein is an epitranscriptomic reader protein. The epitranscriptomic reader protein may include m⁶A Reader Proteins, such as YTHDF1. In some embodiments, the target protein comprises YTHDF1. In some embodiments, the target protein is YTHDF1. As used herein, the term “YTHDF1” refers to “YTH domain-containing family protein 1” or “C20orf21.” In the cytosol, YTHDF1 functions as a “reader” of m6A-modified mRNAs and interacts with initiation factors to facilitate translation initiation.

### Linkers

In some embodiments, the synthetic bifunctional molecule comprises a first domain that specifically binds to an RNA sequence of the target RNA and a second domain that specifically binds to a target polypeptide or protein, wherein the first domain is conjugated to the second domain by a linker molecule.

In certain embodiments, the first domain and the second domain of the bifunctional molecules described herein can be chemically linked or coupled via a chemical linker (L). In certain embodiments, the linker is a group comprising one or more covalently connected structural units. In certain embodiments, the linker directly links the first domain to the second domain. In other embodiments, the linker indirectly links the first domain to the second domain. In some embodiments, one or more linkers can be used to link the first domain and the second domain.

In certain embodiments, the linker is a bond, CR^{L1}R^{L2}, O, S, SO, SO₂, NR^{L3}, SO₂NRL³, SONRL³, CONRL³, NRL³CONR^{w}, NRL³SO₂NR^{w}, CO, CR^{L}═CR^{L2}, C≡C, SiR^{L1}R^{L2}, P(O)R₁, P(O)OR^{L1}, NR^{L3}C(═NCN)NR^{L4}, NR^{L3}C(═NCN), NR^{L3}C(═CNO₂)NR^{L4}, C₃₋₁₁cycloalkyl optionally substituted with 0-6 R^{L1} and/or R^{L2} groups, C₃₋₁₁heterocyclyl optionally substituted with 0-6 R^{L1} and/or R^{L2} groups, aryl optionally substituted with 0-6 RY and/or R^{L2} groups, heteroaryl optionally substituted with 0-6 R^{L1} and/or R^{L2} groups, where RY or R^{L2}, each independently, can be linked to other groups to form cycloalkyl and/or heterocyclyl moeity which can be further substituted with 0-4 R groups; wherein R^{L1}, R^{L2}, R^{L3}, R^{L4} and R^{L5} are, each independently, H, halo, C₁₋₈alkyl, OC₁₋₈alkyl, SC₁₋₈alkyl, NHC₁₋₈alkyl, N(C₁₋₈alkyl)₂, C₃₋₁₁cycloalkyl, aryl, heteroaryl, C₃₋₁₁heterocyclyl, OC₁₋₈cycloalkyl, SC₁₋₈cycloalkyl, NHC₁₋₈cycloalkyl, N(C₁₋₈cycloalkyl)₂, N(C₁₋₈cycloalkyl)(C₁₋₈alkyl), OH, NH₂, SH, SO₂C₁₋₈alkyl, P(O)(OC₁₋₈alkyl)(C₁₋₈alkyl), P(O)(OC₁₋₈alkyl)₂, CC—C₁₋₈alkyl, CCH, CH═CH(C₁₋₈alkyl), C(C₁₋₈alkyl)═CH(C₁₋₈alkyl), C(C₁₋₈alkyl)═C(C₁₋₈alkyl)₂, Si(OH)₃, Si(C₁₋₈alkyl)₃, Si(OH)(C₁₋₈alkyl)₂, COC₁₋₈alkyl, CO₂H, halogen, CN, CF₃, CHF₂, CH₂F, NO₂, SF₅, SO₂NHC₁₋₈alkyl, SO₂N(C₁₋₈alkyl)₂, SONHC₁₋₈alkyl, SON(C₁₋₈alkyl)₂, CONHC₁₋₈alkyl, CON(C₁₋₈alkyl)₂, N(C₁₋₈alkyl)CONH(C₁₋₈alkyl), N(C₁₋₈alkyl)CON(C₁₋₈alkyl)₂, NHCONH(C₁₋₈alkyl), NHCON(C₁₋₈alkyl)₂, NHCONH₂, N(C₁₋₈alkyl)SO₂NH(C₁₋₈alkyl), N(C₁₋₈alkyl)SO₂N(C₁₋₈alkyl)₂, NHSO₂NH(C₁₋₈alkyl), NHSO₂N(C₁₋₈alkyl)₂, NHSO₂NH₂.

In certain embodiments, the linker (L) is selected from the group consisting of:

—(CH₂)ₙ-(lower alkyl)-, —(CH₂)ₙ-(lower alkoxyl)-, —(CH₂)ₙ-(lower alkoxyl) —OCH₂—C(O)—, —(CH₂)ₙ-(lower alkoxyl)-(lower alkyl)-OCH₂—C(O)—, —(CH₂)ₙ-(cycloalkyl)-(lower alkyl)-OCH₂—C(O)—, —(CH₂)ₙ-(hetero cycloalkyl)-, —(CH₂CH₂O)ₙ-(lower alkyl)-O—CH₂—C(O)—,

(CH₂CH₂O)ₙ-(hetero cycloalkyl)-O—CH₂—C(O)—, —(CH₂CH₂O)ₙ-Aryl-O—CH₂—C(O)—, —(CH₂CH₂O)ₙ-(hetero aryl)-O—CH₂—C(O)—, —(CH₂CH₂O)-(cyclo alkyl)-O-(hetero aryl)-O—CH₂—C(O)—, —(CH₂CH₂O)ₙ-(cyclo alkyl)-O-Aryl-O—CH₂—C(O)—, —(CH₂CH₂O)ₙ-(lower alkyl)-NH-Aryl-O—CH₂—C(O)—, —(CH₂CH₂O)ₙ-(lower alkyl)-O-Aryl-C(O)—, —(CH₂CH₂O)ₙ-cycloalkyl-O-Aryl-C(O)—, —(CH₂CH₂O)ₙ-cycloalkyl-O-(hetero aryl)-C(O)—, where n can be 0 to 10;

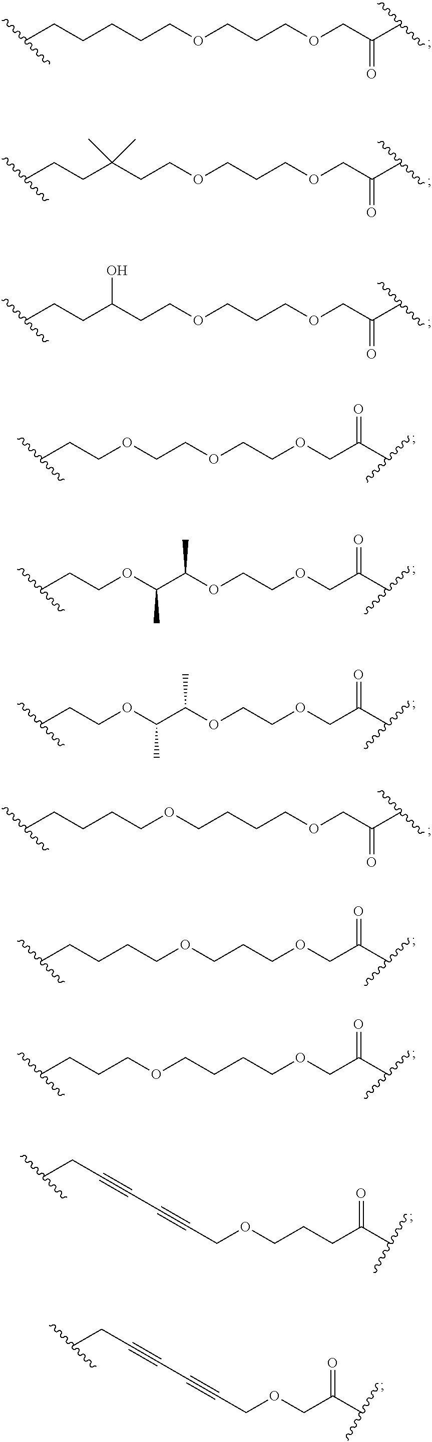

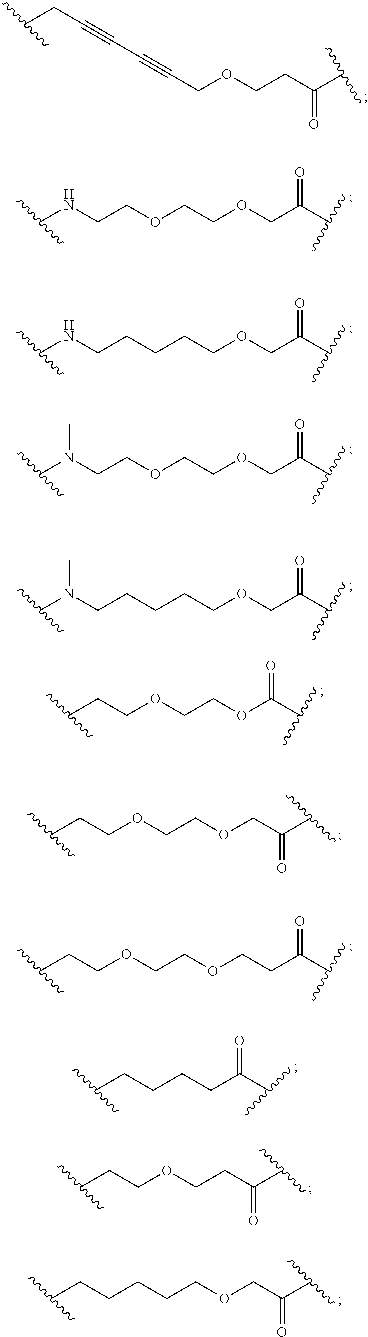

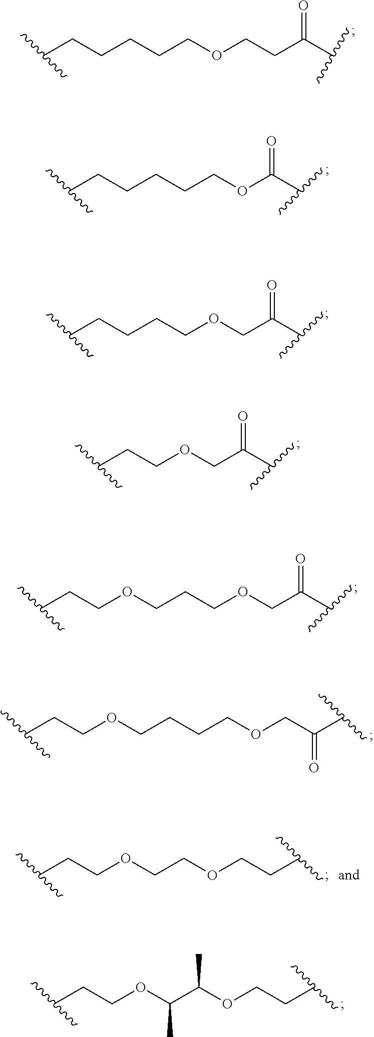

In additional embodiments, the linker group is optionally substituted (poly)ethyleneglycol having between 1 and about 100 ethylene glycol units, between about 1 and about 50 ethylene glycol units, between 1 and about 25 ethylene glycol units, between about 1 and 10 ethylene glycol units, between 1 and about 8 ethylene glycol units and 1 and 6 ethylene glycol units, between 2 and 4 ethylene glycol units, or optionally substituted alkyl groups interdispersed with optionally substituted, O, N, S, P or Si atoms. In certain embodiments, the linker is substituted with an aryl, phenyl, benzyl, alkyl, alkylene, or heterocycle group. In certain embodiments, the linker may be asymmetric or symmetrical.

In any of the embodiments described herein, the linker group may be any suitable moiety as described herein. In one embodiment, the linker is a substituted or unsubstituted polyethylene glycol group ranging in size from about 1 to about 12 ethylene glycol units, between 1 and about 10 ethylene glycol units, about 2 about 6 ethylene glycol units, between about 2 and 5 ethylene glycol units, between about 2 and 4 ethylene glycol units.

Although the first domain and the second domain may be covalently linked to the linker group through any group which is appropriate and stable to the chemistry of the linker, in some aspects, the linker is independently covalently bonded to the first domain and the second domain through an amide, ester, thioester, keto group, carbamate (urethane), carbon or ether, each of which groups may be inserted anywhere on the first domain and second domain to provide maximum binding. In certain preferred aspects, the linker may be linked to an optionally substituted alkyl, alkylene, alkene or alkyne group, an aryl group or a heterocyclic group on the first domain and/or the second domain.

In certain embodiments, the linker can be linear chains with linear atoms from 4 to 24, the carbon atom in the linear chain can be substituted with oxygen, nitrogen, amide, fluorinated carbon, etc., such as the following:

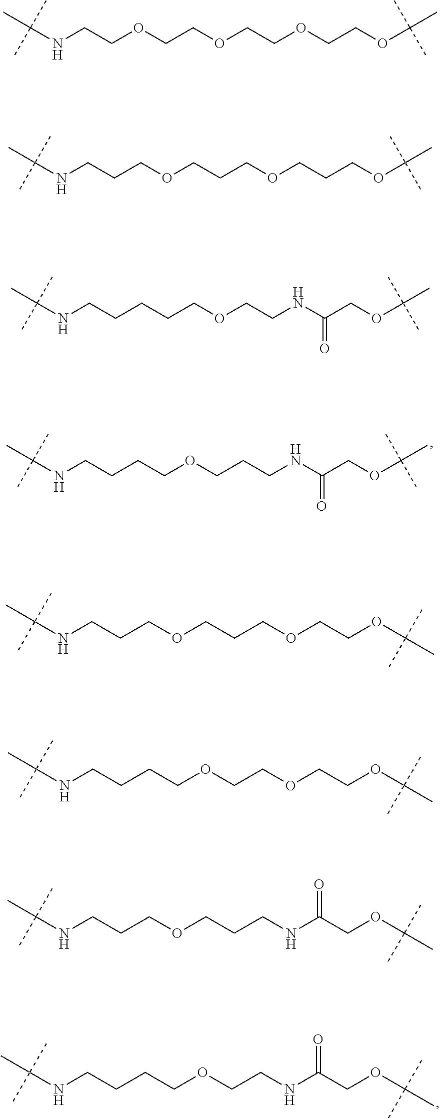

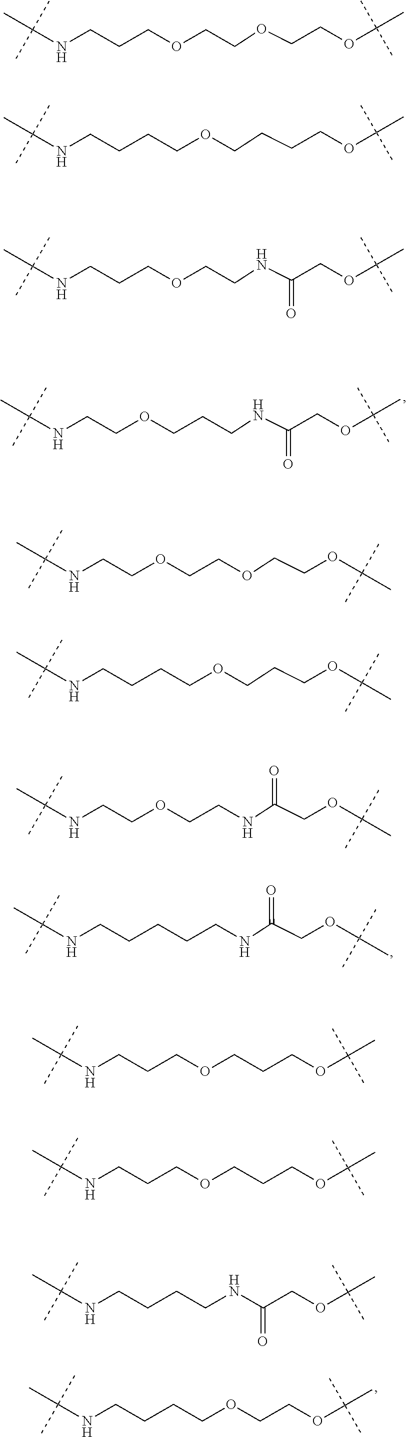

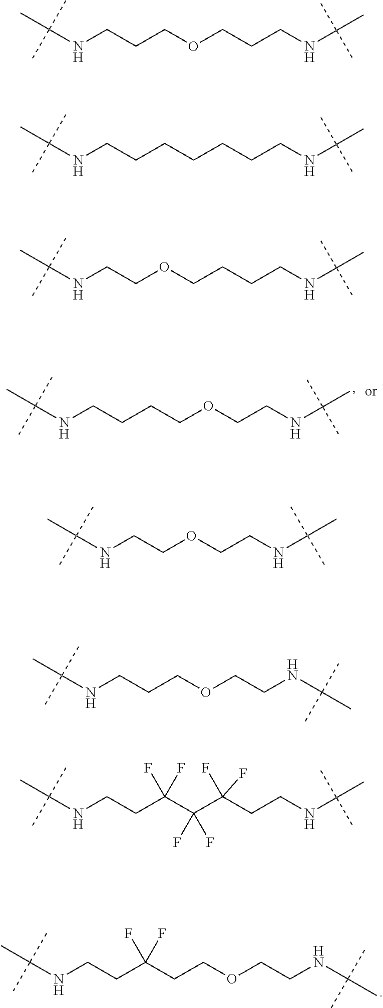

In some embodiments, the linker comprises a TEG linker:

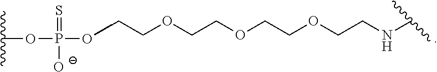

In some embodiments, the linker comprises a mixer of regioisomers. In some embodiments, the mixer of regioisomers is selected from the group consisting of Linkers 1-5:

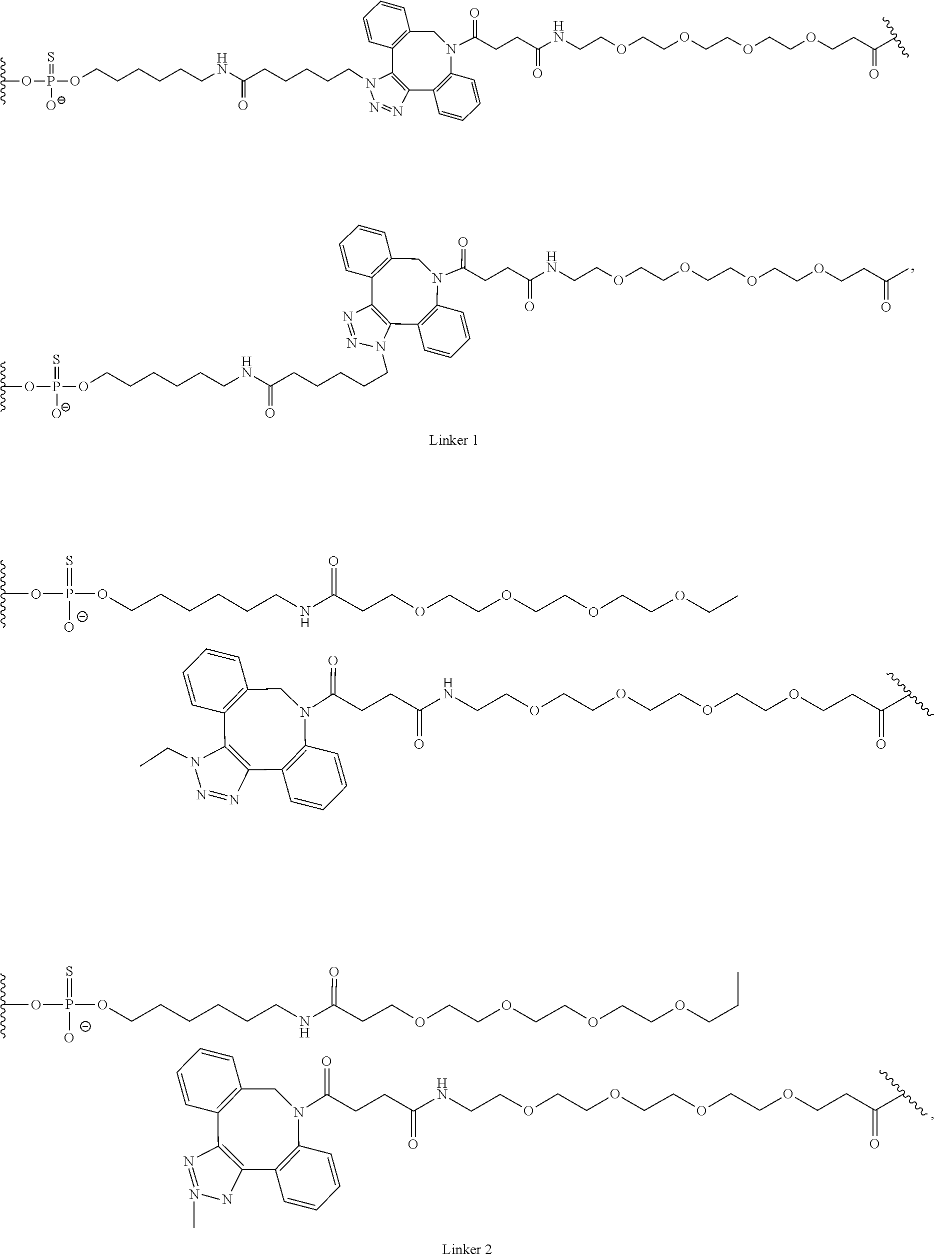

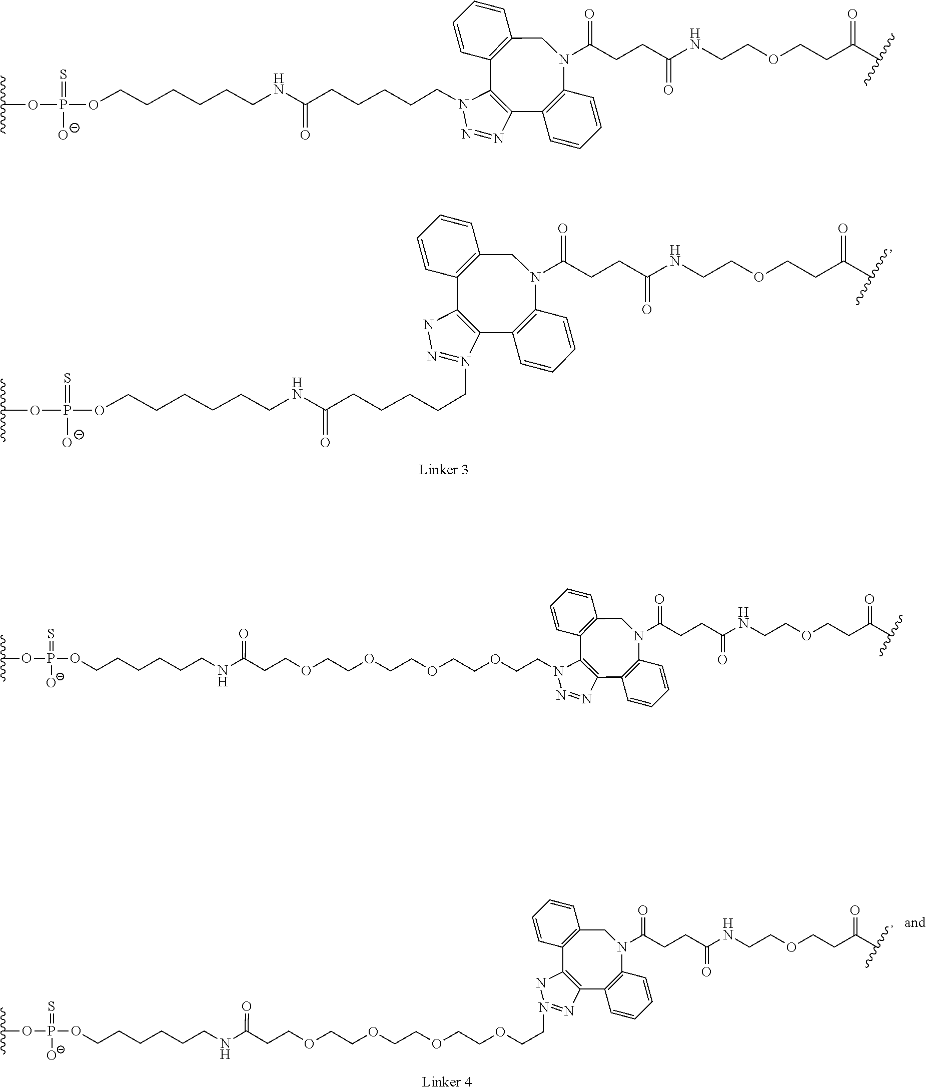

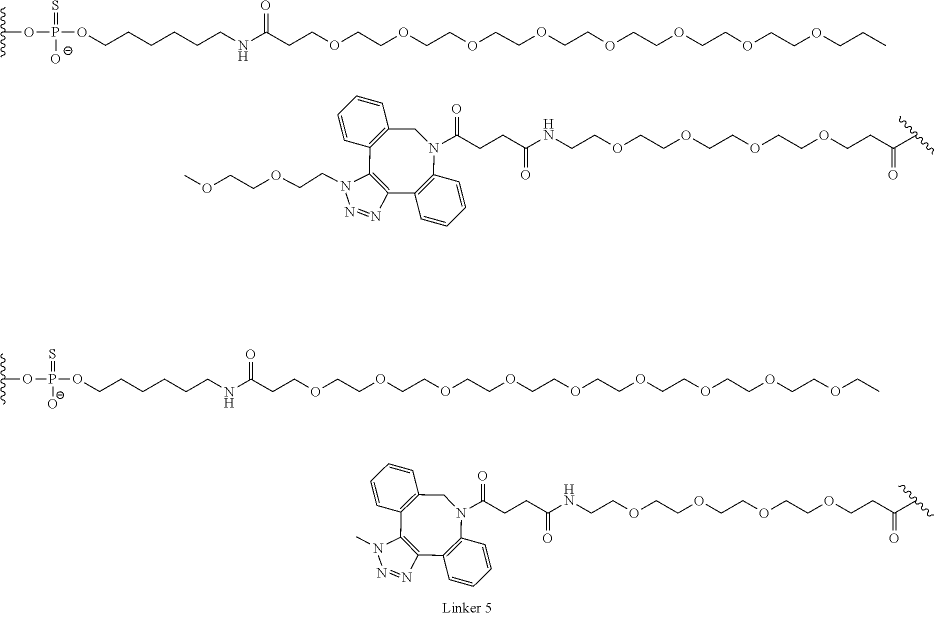

In some embodiments, the linker comprises a modular linker. In some embodiments, the modular linker comprises one or more modular regions that may be substituted with a linker module. In some embodiments, the modular linker having a modular region that can be substituted with a linker module comprises.

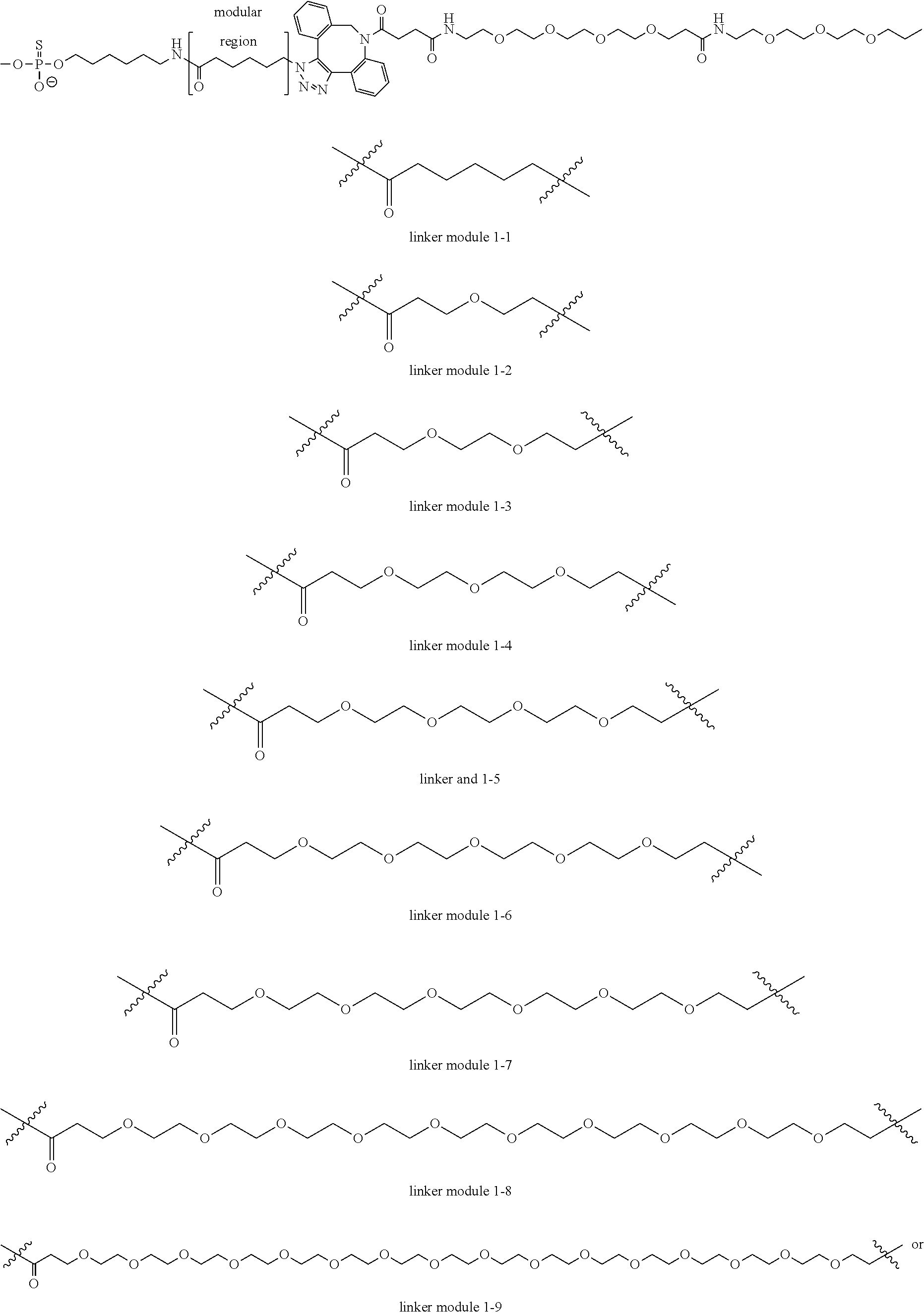

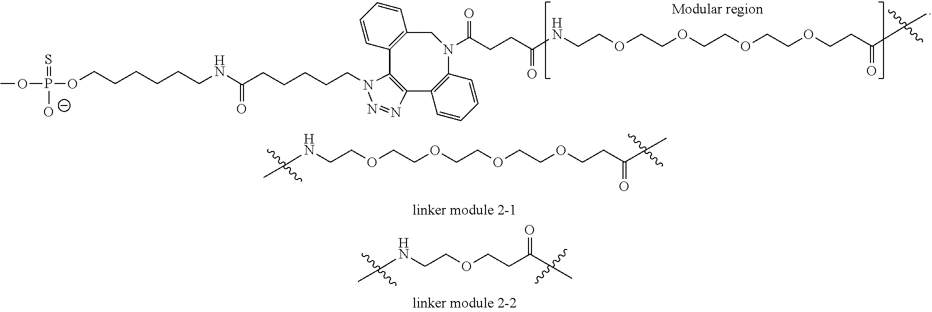

In certain embodiments, the linker can be nonlinear chains, and can be aliphatic or aromatic or heteroaromatic cyclic moieties. Some examples of linkers include but is not limited to the following:

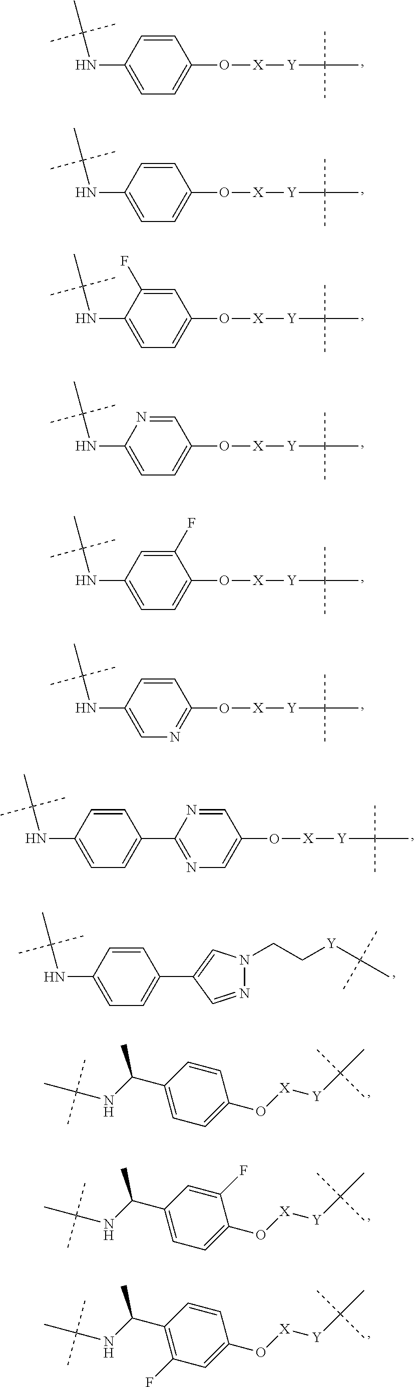

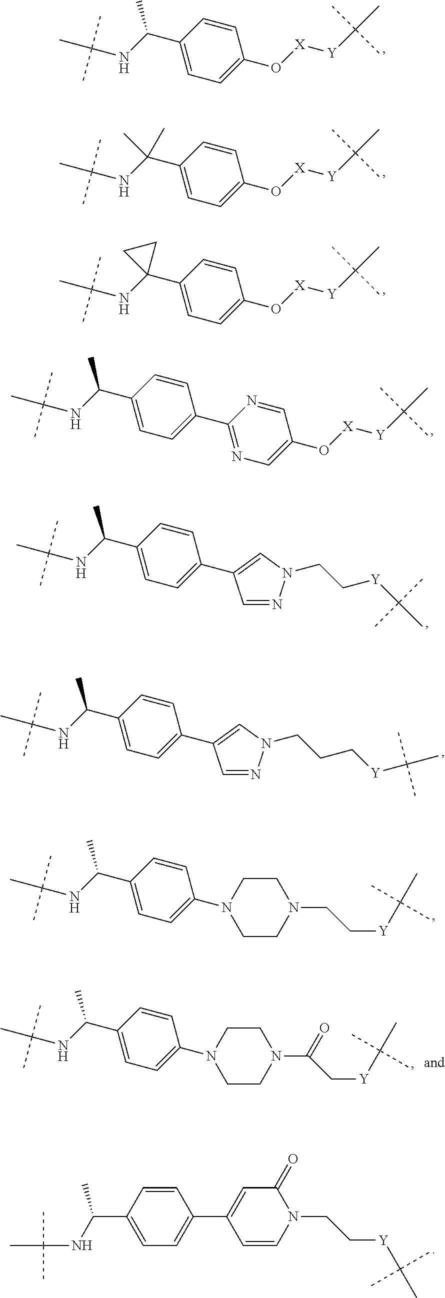

wherein “X” can be linear chain with atoms ranging from 2 to 14, and can contain heteroatoms such as oxygen and “Y” can be O, N, S(O)ₙ (n=0, 1, or 2).

Other examples of linkers include, but are not limited to: Allyl(4-methoxyphenyl)dimethylsilane, 6-(Allyloxycarbonylamino)-1-hexanol, 3-(Allyloxycarbonylamino)-1-propanol, 4-Aminobutyraldehyde diethyl acetal, (E)-N-(2-Aminoethyl)-4-{2-[4-(3-azidopropoxy)phenyl]diazenyl}benzamide hydrochloride, N-(2-Aminoethyl)maleimide trifluoroacetate salt, Amino-PEG4-alkyne, Amino-PEG4-t-butyl ester, Amino-PEG5-t-butyl ester, Amino-PEG6-t-butyl ester, 20-Azido-3,6,9,12,15,18-hexaoxaicosanoic acid, 17-Azido-3,6,9,12,15-pentaoxaheptadecanoic acid, Benzyl N-(3-hydroxypropyl)carbamate, 4-(Boc-amino)-1-butanol, 4-(Boc-amino)butyl bromide, 2-(Boc-amino)ethanethiol, 2-[2-(Boc-amino)ethoxy]ethoxyacetic acid (dicyclohexylammonium) salt, 2-(Boc-amino)ethyl bromide, 6-(Boc-amino)-1-hexanol, 21-(Boc-amino)-4,7,10,13,16,19-hexaoxaheneicosanoic acid purum, 6-(Boc-amino)hexyl bromide, 3-(Boc-amino)-1-propanol, 3-(Boc-amino)propyl bromide, 15-(Boc-amino)-4,7,10,13-tetraoxapentadecanoic acid purum, N-Boc-1,4-butanediamine, N-Boc-cadaverine, N-Boc-ethanolamine, N-Boc-ethylenediamine, N-Boc-2,2′-(ethylenedioxy)diethylamine, N-Boc-1,6-hexanediamine, N-Boc-1,6-hexanediamine hydrochloride, N-Boc-4-isothiocyanatoaniline, N-Boc-3-isothiocyanatopropylamine, N-Boc-N-methylethylenediamine, BocNH-PEG4-acid, BocNH-PEG5-acid, N-Boc-m-phenylenediamine, N-Boc-p-phenylenediamine, N-Boc-1,3-propanediamine, N-Boc-1,3-propanediamine, N-Boc-N′-succinyl-4,7,10-trioxa-1,13-tridecanediamine, N-Boc-4,7,10-trioxa-1,13-tridecanediamine, N-(4-Bromobutyl)phthalimide, 4-Bromobutyric acid, 4-Bromobutyryl chloride, N-(2-Bromoethyl)phthalimide, 6-Bromo-1-hexanol, 8-Bromooctanoic acid, 8-Bromo-1-octanol, 3-(4-Bromophenyl)-3-(trifluoromethyl)-3H-diazirine, N-(3-Bromopropyl)phthalimide, 4-(tert-Butoxymethyl)benzoic acid, tert-Butyl 2-(4-{[4-(3-azidopropoxy)phenyl]azo}benzamido)ethylcarbamate, 2-[2-(tert-Butyldimethylsilyloxy)ethoxy]ethanamine, tert-Butyl 4-hydroxybutyrate, Chloral hydrate, 4-(2-Chloropropionyl)phenylacetic acid, 1,11-Diamino-3,6,9-trioxaundecane, di-Boc-cystamine, Diethylene glycol monoallyl ether, 3,4-Dihydro-2H-pyran-2-methanol, 4-[(2,4-Dimethoxyphenyl)(Fmoc-amino)methyl]phenoxyacetic acid, 4-(Diphenylhydroxymethyl)benzoic acid, 4-(Fmoc-amino)-1-butanol, 2-(Fmoc-amino)ethanol, 2-(Fmoc-amino)ethyl bromide, 6-(Fmoc-amino)-1-hexanol, 5-(Fmoc-amino)-1-pentanol, 3-(Fmoc-amino)-1-propanol, 3-(Fmoc-amino)propyl bromide, N-Fmoc-2-bromoethylamine, N-Fmoc-1,4-butanediamine hydrobromide, N-Fmoc-cadaverine hydrobromide, N-Fmoc-ethylenediamine hydrobromide, N-Fmoc-1,6-hexanediamine hydrobromide, N-Fmoc-1,3-propanediamine hydrobromide, N-Fmoc-N″-succinyl-4,7,10-trioxa-1,13-tridecanediamine, (3-Formyl-1-indolyl)acetic acid, 4-Hydroxybenzyl alcohol, N-(4-Hydroxybutyl)trifluoroacetamide, 4′-Hydroxy-2,4-dimethoxybenzophenone, N-(2-Hydroxyethyl)maleimide, 4-[4-(1-Hydroxyethyl)-2-methoxy-5-nitrophenoxy]butyric acid, N-(2-Hydroxyethyl)trifluoroacetamide, N-(6-Hydroxyhexyl)trifluoroacetamide, 4-Hydroxy-2-methoxybenzaldehyde, 4-Hydroxy-3-methoxybenzyl alcohol, 4-(Hydroxymethyl)benzoic acid, 4-(Hydroxymethyl)phenoxyacetic acid, Hydroxy-PEG4-t-butyl ester, Hydroxy-PEG5-t-butyl ester, Hydroxy-PEG6-t-butyl ester, N-(5-Hydroxypentyl)trifluoroacetamide, 4-(4′-Hydroxyphenylazo)benzoic acid, 2-Maleimidoethyl mesylate, 6-Mercapto-1-hexanol, Phenacyl 4-(bromomethyl)phenylacetate, Propargyl-PEG6-acid, 4-Sulfamoylbenzoic acid, 4-Sulfamoylbutyric acid, 4-(Z-Amino)-1-butanol, 6-(Z-Amino)-1-hexanol, 5-(Z-Amino)-1-pentanol, N—Z-1,4-Butanediamine hydrochloride, N—Z-Ethanolamine, N—Z-Ethylenediamine hydrochloride, N—Z-1,6-hexanediamine hydrochloride, N—Z-1,5-pentanediamine hydrochloride, and N—Z-1,3-Propanediamine hydrochloride.

In some embodiments, the linker is conjugated at a 5′ end or a 3′ end of the ASO. In some embodiments, the linker is conjugated at a position on the ASO that is not at the 5′ end or at the 3′ end.

In some embodiments, linkers comprise 1-10 linker-nucleosides. In some embodiments, such linker-nucleosides are modified nucleosides. In certain embodiments such linker-nucleosides comprise a modified sugar moiety. In some embodiments, linker-nucleosides are unmodified. In some embodiments, linker-nucleosides comprise an optionally protected heterocyclic base selected from a purine, substituted purine, pyrimidine or substituted pyrimidine. In some embodiments, a cleavable moiety is a nucleoside selected from uracil, thymine, cytosine, 4-N-benzoylcytosine, 5-methylcytosine, 4-N -benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue.

In some embodiments, linker-nucleosides are linked to one another and to the remainder of the oligomeric compound through cleavable bonds. In some embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid.

In some embodiments, the linker may be a non-nucleic acid linker. The non-nucleic acid linker may be a chemical bond, e.g., one or more covalent bonds or non-covalent bonds. In some embodiments, the non-nucleic acid linker is a peptide or protein linker. Such a linker may be between 2-30 amino acids, or longer. The linker includes flexible, rigid or cleavable linkers described herein.

In some embodiments, the linker is a single chemical bond (i.e., conjugate moiety is attached to an oligonucleotide via a conjugate linker through a single bond). In some embodiments, the linker comprises a chain structure, such as a hydrocarbyl chain, or an oligomer of repeating units such as ethylene glycol, nucleosides, or amino acid units.

Examples of linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

The most commonly used flexible linkers have sequences consisting primarily of stretches of Gly and Ser residues (“GS” linker). Flexible linkers may be useful for joining domains that require a certain degree of movement or interaction and may include small, non-polar (e.g., Gly) or polar (e.g., Ser or Thr) amino acids. Incorporation of Ser or Thr can also maintain the stability of the linker in aqueous solutions by forming hydrogen bonds with the water molecules, and therefore reduce unfavorable interactions between the linker and the protein moieties.

Rigid linkers are useful to keep a fixed distance between domains and to maintain their independent functions. Rigid linkers may also be useful when a spatial separation of the domains is critical to preserve the stability or bioactivity of one or more components in the fusion. Rigid linkers may have an alpha helix-structure or Pro-rich sequence, (XP)ₙ, with X designating any amino acid, preferably Ala, Lys, or Glu.

Cleavable linkers may release free functional domains in vivo. In some embodiments, linkers may be cleaved under specific conditions, such as the presence of reducing reagents or proteases. In vivo cleavable linkers may utilize the reversible nature of a disulfide bond. One example includes a thrombin-sensitive sequence (e.g., PRS) between the two Cys residues. In vitro thrombin treatment of CPRSC results in the cleavage of the thrombin-sensitive sequence, while the reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in fusions may also be carried out by proteases that are expressed in vivo under pathological conditions (e.g. cancer or inflammation), in specific cells or tissues, or constrained within certain cellular compartments. The specificity of many proteases offers slower cleavage of the linker in constrained compartments.

Examples of linking molecules include a hydrophobic linker, such as a negatively charged sulfonate group; lipids, such as a poly (—CH₂—) hydrocarbon chains, such as polyethylene glycol (PEG) group, unsaturated variants thereof, hydroxylated variants thereof, amidated or otherwise N-containing variants thereof, noncarbon linkers; carbohydrate linkers; phosphodiester linkers, or other molecule capable of covalently linking two or more polypeptides. Non-covalent linkers are also included, such as hydrophobic lipid globules to which the polypeptide is linked, for example through a hydrophobic region of the polypeptide or a hydrophobic extension of the polypeptide, such as a series of residues rich in leucine, isoleucine, valine, or perhaps also alanine, phenylalanine, or even tyrosine, methionine, glycine or other hydrophobic residue. The polypeptide may be linked using charge-based chemistry, such that a positively charged moiety of the polypeptide is linked to a negative charge of another polypeptide or nucleic acid.

In some embodiments, a linker comprises one or more groups selected from alkyl, amino, oxo, amide, disulfide, polyethylene glycol, ether, thioether, and hydroxylamino. In certain such embodiments, the linker comprises groups selected from alkyl, amino, oxo, amide and ether groups. In some embodiments, the linker comprises groups selected from alkyl and amide groups. In some embodiments, the linker comprises groups selected from alkyl and ether groups. In some embodiments, the linker comprises at least one phosphorus moiety. In some embodiments, the linker comprises at least one phosphate group. In some embodiments, the linker includes at least one neutral linking group.

In some embodiments, the linkers are bifunctional linking moieties, e.g., those known in the art to be useful for attaching conjugate groups to oligomeric compounds, such as the ASOs provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In some embodiments, bifunctional linking moieties comprise one or more groups selected from amino, hydroxyl, carboxylic acid, thiol, alkyl, alkenyl, and alkynyl.

### Target Protein (Effector) Function

In some embodiments, the bifunctional molecule comprises a second domain that specifically binds to a target protein. In some embodiments, the target protein is an effector. In some embodiments, the target protein is an endogenous protein. In other embodiments, the target protein is an intracellular protein. In another embodiment, the target protein is an endogenous and intracellular protein. In some embodiments, the target endogenous protein is an enzyme, scaffolding protein or a regulatory protein. In some embodiments, the second domain specifically binds to an active site, an allosteric site, or an inert site on the target polypeptide or protein.

### Increasing RNA Translation

In some embodiments, the second domain of the bifunctional molecules as provided herein targets a protein that is involved in increasing translation of a ribonucleic acid sequence in a transcript of a gene from Table 3. In some embodiments, the second domain of the bifunctional molecules as provided herein targets a protein that increases translation of a ribonucleic acid in a transcript of a gene from Table 3. In some embodiments, the first domain of the bifunctional molecules as provided herein targets a ribonucleic acid sequence in a transcript of a gene from Table 3, thereby increasing translation of a target ribonucleic acid sequence. In some embodiments, the first domain of the bifunctional molecules as provided herein binds to one or more ribonucleic acid sequences that are proximal or near to a sequence that mediates an increase in translation of a ribonucleic acid molecule of a gene from Table 3. In some embodiments, the ribonucleic acid molecule is associated with a tumor suppressor gene. In some embodiments, the ribonucleic acid molecule is associated with haploinsufficiency.

TABLE 3
Exemplary Genes whose RNA transcript is increasingly translated by a
Bifunctional Molecule

Neoplasia PTEN; ATM; ATR; EGFR; ERBB2; ERBB3; ERBB4; Notch1; Notch2; Notch3;
Notch4; AKT; AKT2; AKT3; HIF; HIF1a; HIF3a; Met; HRG; Bcl2; PPAR alpha; PPAR
gamma; WT1 (Wilms Tumor); FGF Receptor Family members (5 members: 1, 2, 3, 4, 5);
CDKN2a; APC; RB (retinoblastoma); MEN1; VHL; BRCA1; BRCA2; AR (Androgen
Receptor); TSG101; IGF; IGF Receptor; Igf1 (4 variants); Igf2 (3 variants); Igf 1 Receptor; Igf
2 Receptor; Bax; Bcl2; caspases family (9 members: 1, 2, 3, 4, 6, 7, 8, 9, 12); Kras; Apc Age-
related Macular Aber; Ccl2; Cc2; cp (ceruloplasmin); Timp3; cathepsinD; Degeneration Vldlr;
Ccr2 Schizophrenia Neuregulin1 (Nrg1); Erb4 (receptor for Neuregulin); Complexin1 (Cplx1);
Tph1 Tryptophan hydroxylase; Tph2 Tryptophan hydroxylase 2; Neurexin 1; GSK3; GSK3a;
GSK3b Disorders 5-HTT (Slc6a4); COMT; DRD (Drd1a); SLC6A3; DAOA; DTNBP1; Dao
(Dao1) Trinucleotide Repeat HTT (Huntington's Dx); SBMA/SMAX1/AR (Kennedy’s
Disorders Dx); FXN/X25 (Friedrich's Ataxia); ATX3 (Machado-Joseph's Dx); ATXN1 and
ATXN2 (spinocerebellar ataxias); DMPK (myotonic dystrophy); Atrophin-1 and Atn1
(DRPLA Dx); CBP (Creb-BP-global instability); VLDLR (Alzheimer's); Atxn7; Atxn10
Fragile X Syndrome FMR2; FXR1; FXR2; mGLUR5 Secretase Related APH-1 (alpha and
beta); Presenilin (Psen1); nicastrin Disorders (Ncstn); PEN-2 Others Nos1; Parp1; Nat1; Nat2
Prion-related disorders Prp ALS SOD1; ALS2; STEX; FUS; TARDBP; VEGF (VEGF-a;
VEGF-b; VEGF-c) Drug addiction Prkce (alcohol); Drd2; Drd4; ABAT (alcohol); GRIA2;
Grm5; Grin1; Htr1b; Grin2a; Drd3; Pdyn; Gria1 (alcohol) Autism Mecp2; BZRAP1; MDGA2;
Sema5A; Neurexin 1; Fragile X (FMR2 (AFF2); FXR1; FXR2; Mglur5) Alzheimer's Disease
E1; CHIP; UCH; UBB; Tau; LRP; PICALM; Clusterin; PS1; SORL1; CR1; Vldlr; Uba1;
Uba3; CHIP28 (Aqp1, Aquaporin 1); Uchl1; Uchl3; APP Inflammation IL-10; IL-1 (IL-1a; IL-
1b); IL-13; IL-17 (IL-17a (CTLA8); IL-17b; IL-17c; IL-17d; IL-17f); II-23; Cx3cr1; ptpn22;
TNFa; NOD2/CARD15 for IBD; IL-6; IL-12 (IL-12a; IL-12b); CTLA4; Cx3cl1 Parkinson's
Disease x-Synuclein; DJ-1; LRRK2; Parkin; PINK1; OPA1; SCN1A; MECP2

In some embodiments, the target proteins are effectors involved in promoting, boosting, increasing RNA translation. For example, such boosters include, but not limited to, translation initiation factors; Cap binding proteins (CBP); DEAD helicase; UBX; and MAP kinase. In some embodiments, the target protein is a translation initiation factor. In some embodiments, the target protein is CBP.

Exemplary boosters may also include EIF4A; EIF4G; EIF4E; DDX1; SLBP; IDH1; G3BP2; RPLP0; YWHAE; YTHDFT; LARP1; BOLL; PAIP; APOBEC3F; CLK2; RPUSD3; PTPB1; NUSAPI; THOC1; MTDH; PEG10; PRPF3; DAZ4; ZRANB2; SRSF8; PABP; YTHDF3; METTL3; ABCF1; P97; P86; EIF3A; EIF3B; EIF3C; EIF3D; EIF3E; EIF3F; EIF3G; EIF3H; EIF3I; EIF3J; EIF3K; EIF3L; EIF3M; APOBEC3F; CLK2; UBAP2L; ZCCH6; CLK3; HSPB1; SRSF8; and ZRANB2. In additional embodiments, the booster is selected from the group consisting of EIF4A; EIF4G; EIF4E; DDX1; SLBP; IDH1; G3BP2; RPLP0; YWHAE; YTHDF1; and LARP1. In additional embodiments, the booster is EIF4A. In additional embodiments, the booster is EIF4G. In additional embodiments, the booster is DDX1. In additional embodiments, the booster is SLBP. In additional embodiments, the booster is IDH1. In additional embodiments, the booster is G3BP2. In additional embodiments, the booster is RPLP0. In additional embodiments, the booster is YWHAE. In additional embodiments, the booster is LARP1.

In some embodiments, the target protein involved in RNA translation, e.g., eIF4E, is recruited to the target RNA by interaction with the target protein bound to the bifunctional molecule as provided herein and mediates promotion of target RNA translation.

In some embodiments, the target proteins may be enzymes. In some embodiments, the target proteins may be receptors. In some embodiments, the target proteins may be signaling proteins or peptides. In some embodiments, the target proteins may be translation factors. In some embodiments, the target proteins may be translational regulators or mediators. In some embodiments, the target proteins may recruit translation factors, translational regulators or translational mediators.

In some embodiments, the target protein comprises a translational regulator. In some embodiments, the target protein comprises a translational promoter.

In some embodiments, the first domain recruits the bifunctional molecule as described herein to the target site by binding to the target RNA, in which the second domain interacts with the target protein and promotes RNA translation. In some embodiments, the target protein after interacts with the second domain of the bifunctional molecule as provided herein further recruits proteins or peptides involved in RNA translation through interaction with the proteins or peptides.

In some embodiments, translation of the ribonucleic acid sequence is upregulated or increased. In some embodiments, translation the ribonucleic acid sequence is increased.

In some embodiments, the bifunctional molecule as provided herein recruits a protein and promotes translation of a ribonucleic acid sequence. By recruiting enzymes or proteins to a target RNA, the local concentration of the enzyme or protein near the transcript is increased, thereby increasing translation of the RNA transcripts (e.g., activating translation of the transcripts).

In some embodiments, the first domain recruits the bifunctional molecule as described herein to the target site by binding to the target RNA or gene sequence, in which the second domain interacts with the target protein and increase translation of the target RNA. In some embodiments, the target protein recruits the bifunctional molecule as described herein by binding to the second domain of the bifunctional molecule as provided herein, in which the first domain specifically binds to a target RNA or another gene sequence, and increases translation of the target RNA. In some embodiments, the target protein after interacting with the second domain of the bifunctional molecule as provided herein further recruits proteins or peptides involved in increasing RNA translation through interaction with the proteins or peptides.

### Pharmaceutical Compositions

In some aspects, the bifunction molecules described herein comprises pharmaceutical compositions, or the composition comprising the bifunctional molecule as described herein.

In some embodiments, the pharmaceutical composition further comprises a pharmaceutically acceptable excipient. Pharmaceutical compositions may be sterile and/or pyrogen-free. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals, e.g., pet and live-stock animals, such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

The term “pharmaceutical composition” is intended to also disclose that the bifunctional molecules as described herein comprised within a pharmaceutical composition can be used for the treatment of the human or animal body by therapy. It is thus meant to be equivalent to the “bifunctional molecule as described herein for use in therapy.”

### Delivery

Pharmaceutical compositions as described herein can be formulated for example to include a pharmaceutical excipient. A pharmaceutical carrier may be a membrane, lipid bilayer, and/or a polymeric carrier, e.g., a liposome or particle such as a nanoparticle, e.g., a lipid nanoparticle, and delivered by known methods to a subject in need thereof (e.g., a human or non-human agricultural or domestic animal, e.g., cattle, dog, cat, horse, poultry). Such methods include, but not limited to, transfection (e.g., lipid-mediated, cationic polymers, calcium phosphate); electroporation or other methods of membrane disruption (e.g., nucleofection), fusion, and viral delivery (e.g., lentivirus, retrovirus, adenovirus, AAV).

In some aspects, the methods comprise delivering the bifunctional molecule as described herein, the composition comprising the bifunctional molecule as described herein, or the pharmaceutical compositions comprising the bifunctional molecule as described herein to a subject in need thereof.

### Methods of Delivery

A method of delivering the bifunctional molecule as described herein, the composition comprising the bifunctional molecule as described herein, or the pharmaceutical compositions comprising the bifunctional molecule as described herein to a cell, tissue, or subject, comprises administering the bifunctional molecule as described herein, the composition comprising the bifunctional molecule as described herein, or the pharmaceutical compositions comprising the bifunctional molecule as described herein to the cell, tissue, or subject.

In some embodiments the bifunctional molecule as described herein, the composition comprising the bifunctional molecule as described herein, or the pharmaceutical compositions comprising the bifunctional molecule as described herein is administered parenterally. In some embodiments the bifunctional molecule as described herein, the composition comprising the bifunctional molecule as described herein, or the pharmaceutical compositions comprising the bifunctional molecule as described herein is administered by injection. The administration can be systemic administration or local administration. In some embodiments, the bifunctional molecule as described herein, the composition comprising the bifunctional molecule as described herein, or the pharmaceutical compositions comprising the bifunctional molecule as described herein is administered intravenously, intraarterially, intraperitoneally, intradermally, intracranially, intrathecally, intralymphaticly, subcutaneously, or intramuscularly.

In some embodiments, the cell is a eukaryotic cell. In some embodiments, the cell is a mammalian cell. In some embodiments, the cell is a human cell. In some embodiments, the cell is an animal cell.

### Methods Using Bifunctional Molecules

### Method of Translating a Ribonucleic Acid Sequence

In some embodiments, the target polypeptide or protein modulates RNA translation.

In some embodiments, the second domain of the bifunctional molecules as provided herein targets a protein that translates a ribonucleic acid sequence in a transcript of a gene from Table 3.

In some embodiments, translation of a gene transcript is upregulated or increased. In some embodiments, translation of a gene transcript is upregulated. In some embodiments, translation of a gene transcript is increased.

In some aspects, a method of translation of a ribonucleic acid sequence in a cell comprises administering to a cell a synthetic bifunctional molecule comprising a first domain comprising an antisense oligonucleotide (ASO) or a small molecule that specifically binds to a target ribonucleic acid sequence or structure, a second domain that specifically binds to a target polypeptide or protein and a linker that conjugates the first domain to the second domain, wherein the target polypeptide or protein translates the ribonucleic acid sequence in the cell.

In one aspect, the method of translating a ribonucleic acid sequence in a cell comprises administering to a cell the synthetic bifunctional molecule as provided herein.

In some embodiments, the second domain comprising a small molecule or an aptamer.

In some embodiments, the cell is a human cell. In some embodiments, the human cell is infected with a virus. In some embodiments, the cell is a cancer cell. In some embodiments, the cell is a bacterial cell.

In some embodiments, the first domain is conjugated to the second domain by a linker molecule.

In some embodiments, the first domain is an antisense oligonucleotide.

In some embodiments, the first domain is a small molecule. In some embodiments, the small molecule is selected from the group consisting of Table 2. In some embodiments, the second domain is a small molecule.

In some embodiments, the second domain is an aptamer.

In some embodiments, the target polypeptide or protein is an intracellular protein. In some embodiments, the target polypeptide or protein is an enzyme, scaffolding protein or a regulatory protein. In some embodiments, the second domain specifically binds to an active site, an allosteric site, or an inert site on the target polypeptide or protein.

In some embodiments, the target proteins are effectors involved in promoting, boosting, increasing RNA translation. For example, such boosters include, but not limited to, EIF4A; EIF4G; EIF4E; DDX1; SLBP; IDH1; G3BP2; RPLP0; YWHAE; YTHDF1; LARP1; BOLL; PAIP; APOBEC3F; CLK2; RPUSD3; PTPB1; NUSAPI; THOC1; MTDH; PEG10; PRPF3; DAZ4; ZRANB2; SRSF8; PABP; YTHDF3; METTL3; ABCF1; P97; P86; EIF3A; EIF3B; EIF3C; EIF3D; EIF3E; EIF3F; EIF3G; EIF3H; EIF3I; EIF3J; EIF3K; EIF3L; EIF3M; APOBEC3F; CLK2; UBAP2L; ZCCH6; CLK3; HSPB1; SRSF8; and ZRANB2. In additional embodiments, the booster is EIF4E. In additional embodiments, the booster is EIF4A. In additional embodiments, the booster is EIF4G. In additional embodiments, the booster is YTHDF1.

Modulation of molecules may be measured by conventional assays known to a person of skill in the art, including, but not limited to, measuring protein levels by, e.g., immunoblot.

In some embodiments, the target protein is the protein involved in RNA translation, e.g., eIF4E, and when recruited to the target RNA by interaction with the second domain of the bifunctional molecule as provided herein, mediates translation of the target RNA. In some embodiments, the target protein is the protein that increases RNA translation and when recruited to the target RNA by interaction with the second domain of the bifunctional molecule as provided herein, increases RNA translation. In some embodiments, the protein involved in RNA translation, e.g., eIF4E, is recruited to the target RNA as provided herein and mediates translation of the target RNA.

In some embodiments, target RNA translation is increased.

In some embodiments, RNA translation is increased by at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, at least 2000%, at least 3000%, at least 4000%, at least 5000%, at least 6000%, at least 7000%, at least 8000%, at least 9000%, at least 10000%, at least 20000%, at least 30000%, at least 40000%, at least 50000%, at least 60000%, at least 70000%, at least 80000%, at least 90000%, or at least 100000% as compared to an untreated control cell, tissue or subject, or compared to the corresponding activity in the same type of cell, tissue or subject before treatment with the modulator as measured by any standard technique. In some embodiments, RNA translation is increased by at least 2 fold, at least 3 fold, at least 4 fold, at least 5 fold, at least 10 fold, at least 20 fold, at least 25 fold, at least 30 fold, at least 40 fold, at least 50 fold, at least 60 fold, at least 70 fold, at least 80 fold, at least 90 fold, at least 100 fold, at least 200 fold, at least 300 fold, at least 400 fold, at least 500 fold, at least 600 fold, at least 700 fold, at least 800 fold, at least 900 fold, at least 1000 fold, at least 2000 fold, at least 3000 fold, at least 4000 fold, at least 5000 fold, at least 6000 fold, at least 7000 fold, at least 8000 fold, at least 9000 fold, or at least 10000 fold as compared to an untreated control cell, tissue or subject, or compared to the corresponding activity in the same type of cell, tissue or subject before treatment with the modulator as measured by any standard technique.

In some embodiments, the bifunctional molecule as provided herein may be used in combination of a fusion protein of a protein domain binding to the second domain and a protein involved in RNA translation, e.g., eIF4E. In some embodiments, recruitment of eIF4E by the bifunctional molecule as provided herein may promote target RNA translation.

### Methods of Treatment

The bifunctional molecules as described herein can be used in a method of treatment for a subject in need thereof. A subject in need thereof, for example, has a disease or condition. In some embodiments, the disease is a cancer, a metabolic disease, an inflammatory disease, a cardiovascular disease, an infectious disease, a genetic disease, a haploinsufficiency disease, or a neurological disease. In some embodiments, the disease is a cancer and wherein the target gene is an oncogene. In some embodiments, the gene of which translation is increased by the bifunctional molecule as provided herein or the composition comprising the bifunctional molecule as provided herein is associated with a disease from Table 4.

TABLE 4
Exemplary Diseases (and associated genes) for treatment with a Bifunctional
Molecule

Blood and Anemia (CDAN1, CDA1, RPS19, DBA, PKLR, PK1, NT5C3, UMPH1, coagulation diseases
PSN1, RHAG, RH50A, NRAMP2, SPTB, ALAS2, ANH1, ASB, and disorders ABCB7, ABC7,
ASAT); Bare lymphocyte syndrome (TAPBP, TPSN, TAP2, ABCB3, PSF2, RING11, MHC2TA,
C2TA, RFX5, REXAP, RFX5), Bleeding disorders (TBXA2R, P2RX1, P2X1); Factor H and factor H-
like 1 (HF1, CFH, HUS); Factor V and factor VIII (MCFD2); Factor VII deficiency (F7); Factor X
deficiency (F10); Factor XI deficiency (F11); Factor XII deficiency (F12, HAF); Factor XIIIA
deficiency (F13A1, F13A); Factor XIIIB deficiency (F13B); Fanconi anemia (FANCA, FACA, FA1,
FA, FAA, FAAP95, FAAP90, FLJ34064, FANCB, FANCC, FACC, BRCA2, FANCD1, FANCD2,
FANCD, FACD, FAD, FANCE, FACE, FANCF, XRCC9, FANCG, BRIP1, BACH1, FANCJ, PHF9,
FANCL, FANCM, KIAA1596); Hemophagocytic lymphohistiocytosis disorders (PRF1, HPLH2,
UNC13D, MUNC13-4, HPLH3, HLH3, FHL3); Hemophilia A (F8, F8C, HEMA); Hemophilia B (F9,
HEMB), Hemorrhagic disorders (PI, ATT, F5); Leakocyde deficiencies and disorders (ITGB2, CD18,
LCAMB, LAD, EIF2B1, EIF2BA, EIF2B2, EIF2B3, EIF2B5, LVWM, CACH, CLE, EIF2B4) Sickle
cell anemia (HBB); Thalassemia (HBA2, HBB, HBD, LCRB, HBA1)
Cell dysregulation B-cell non-Hodgkin lymphoma (BCL7A, BCL7); Leukemia (TAL1 and oncology
TCL5, SCL, TAL2, FLT3, NBS1, NBS, ZNFN1A1, IK1, LYF1, diseases and disorders HOXD4,
HOX4B, BCR, CML, PHL ALL, ARNT, KRAS2, RASK2, GMPS, AF10, ARHGEF12, LARG,
KIAA0382, CALM, CLTH, CEBPA, CEBP, CHIC2, BTL, FLT3, KIT, PBT, EPP, NPM1, NUP214,
D9S46E, CAN, CAIN, RUNX1, CBPA2, AML1, WHSC1L1, NSD3, FLT3, AF1Q, NPM1, NUMA1,
ZNF145, PLZF, PML, MYL, STAT5B, AF10, CALM, CLTH, ARL11, ARLTS1, P2RX7, P2X7, BCR,
CML, PHL, ALL, GRAF, NF1, VRNF, WSS, NFNS, PTPN11, PTP2C, SHP2, NS1, BCL2, CCND1,
PRAD1, BCL1, TCRA, GATA1, GF1, ERYF1, NFE1, ABL1, NQO1, DIA4, NMOR1, NUP214,
D9S46E, CAN, CAIN), Inflammation and AIDS (KIR3DL1, NKAT3, NKB1, AMB11, KIR3DS1,
IFNG, CXCL12, immune related SDF1); Autoimmune lymphoproliferative syndrome (TNFRSF6,
APT1, diseases and disorders FAS, CD95, ALPS1A); Combined immunodeficiency, (IL2RG, SCIDX1,
SCIDX, IMD4); HIV-1 (CCL5, SCYA5, D17S136E, TCP228), HIV susceptibility or infection (IL10,
CSIF, CMKBR2, CCR2, CMKBR5, CCCKR5 (CCR5)); Immunodeficiencies (CD3E, CD3G, AICDA,
AID, HIGM2, TNFRSF5, CD40, UNG, DGU, HIGM4, TNFSF5, CD40LG, HIGM1, IGM, FOXP3,
IPEX, AIID, XPID, PIDX, TNFRSF14B, TAC1); Inflammation (IL-10, IL-1 (IL-1a, IL-1b), IL-13, IL-
17 (IL-17a (CTLA8), IL-17b, IL-17c, IL-17d, IL-17f), II-23, Cx3cr1, ptpa22, TNFa, NOD2/CARD15
for IBD, IL-6, IL-12 (IL-12a, IL-12b), CTLA4, Cx3cl1); Severe combined immunodeficiencies
(SCIDs)(JAK3, JAKL, DCLRE1C, ARTEMIS, SCIDA, RAG1, RAG2, ADA, PTPRC, CD45, LCA,
IL7R, CD3D, T3D, IL2RG, SCIDX1, SCIDX, IMD4).
Metabolic, liver, Amyloid neuropathy (TTR, PALB); Amyloidosis (APOA1, APP, AAA, kidney and
protein CVAP, AD1, GSN, FGA, LYZ, TTR, PALB); Cirrhosis (KRT18, KRT8, diseases and
disorders CIRH1A, NAIC, TEX292, KIAA1988); Cystic fibrosis (CFTR, ABCC7, CF, MRP7);
Glycogen storage diseases (SLC2A2, GLUT2, G6PC, G6PT, G6PT1, GAA, LAMP2, LAMPB, AGL,
GDE, GBE1, GYS2, PYGL, PFKM); Hepatic adenoma, 142330 (TCF1, HNF1A, MODY3), Hepatic
failure, early onset, and neurologic disorder (SCOD1, SCO1), Hepatic lipase deficiency (LIPC),
Hepatoblastoma, cancer and carcinomas (CTNNB1, PDGFRL, PDGRL, PRLTS, AXIN1, AXIN,
CTNNB1, TP53, P53, LFS1, IGF2R, MPRI, MET, CASP8, MCH5; Medullary cystic kidney disease
(UMOD, HNFJ, FJHN, MCRD2, ADMCKD2): Phenylketonuria (PAH, PKU1, QDPR, DHPR, PTS);
Polycystic kidney and hepatic disease (FCYT, PKHD1, ARPKD, PKD1, PKD2, PKD4, PKDTS,
PRKCSH, G19P1, PCLD, SEC63).
Muscular/Skeletal Becker muscalar dystrophy (DMD, BMD, MYF6), Duchenne Muscular diseases and
disorders Dystrophy (DMD, BMD); Emery-Dreifuss muscular dystrophy (LMNA, LMN1, EMD2,
FPLD, CMD1A, HGPS, LGMD1B, LMNA, LMN1, EMD2, FPLD, CMD1A) Facioscapulohumeral
muscular dystrophy (FSHMD1A, FSHD1A); Muscular dystrophy (FKRP, MDC1C, LGMD2I, LAMA2,
LAMM, LARGE, KTAA0609, MDC1D, FCMD, TTID, MYOT, CAPN3, CANP3, DYSF, LGMD2B,
SCCG, LGMD2C, DMDA1, SCG3, SGCA, ADL, DAG2, LGMD2D, DMDA2, SGCB, LGMD2E,
SGCD, SGD, LGMD2F, CMD1L, TCAP, LGMD2G, CMD1N, TRIM32, HT2A, LGMD2H, FKRP,
MDC1C, LGMD2I, TTN, CMD1G, TMD, LGMD2J, POMT1, CAV3, LGMD1C, SEPN1, SELN,
RSMD1, PLEC1, PLTN, EBS1); Osteopetrosis (LRP5, BMND1, LRP7, LR3, OPPG, VBCH2, CLCN7,
CLC7, OPTA2, OSTM1, GL, TCIRG1, TIRC7, OC116, OPTB1); Muscular atrophy (VAPB, VAPC,
ALS8, SMN1, SMA1, SMA2, SMA3, SMA4, BSCL2, SPG17, GARS, SMAD1, CMT2D, HEXB,
IGHMBP2, SMUBP2, CATF1, SMARD1)
Neurological and ALS (SOD1, ALS2, STEX, FUS, TARDBP, VEGF (VEGF-a, VEGF-b, neuronal
diseases and VEGF-c): Alzheimer disease (APP, AAA, CVAP, AD1, APOE, AD2, disorders PSEN2,
AD4, STM2, APBB2, FE65L1, NOS3, PLAU, URK, ACE, DCP1, ACE1, MPO, PACIP1, PAXIP1L,
PTIP, A2M, BLMH, BMH, PSEN1, AD3); Autism (Mecp2, BZRAP1, MDGA2, Sema5A, Neurexin1,
GLO1, MECP2, RTT, PPMX, MRX16, MRX79, NLGN3, NLGN4, KIAA1260, AUTSX2); Fragile X
Syndrome (FMR2, FXR1, FXR2, mGLUR5); Huntington's disease and disease like disorders (HD,
IT15, PRNP, PRIP, JPH3, JP3, HDL2, TBP, SCA17); Parkinson disease (NR4A2, NURR1, NOT,
TINUR, SNCAIP, TBP, SCA17, SNCA, NACP, PARK1, PARK4, DJ1, PARK7, LRRK2, PARK8,
PINK1, PARK6, UCHL1, PARK5, SNCA, NACP, PARK1, PARK4, PRKN, PARK2, PDJ, DBH,
NDUFV2); Rett syadrome (MECP2, RTT, PPMX, MRX16, MRX79, CDKL5, STK9, MECP2, RTT,
PPMX, MRX16, MRX79, x-Synuclein, DJ-1); Schizophrenia (Neuregulin1 (Nrg1), Erb4 (receptor for
Neuregulin), Complexin1 (Cplx1), Tph1 Tryptophan hydroxylase, Tph2, Tryptophan hydroxylase 2,
Neurexin 1, GSK3, GSK3a, GSK3b, 5-HTT (Slc6a4), COMT, DRD (Drd1a), SLC6A3, DAOA,
DTNBP1, Dao (Dao1)); Secretase Related Disorders (APH-1 (alpha and beta), Presenilin (Psen1),
nicastrin, (Ncstn), PEN-2, Nos1, Parp1, Nat1, Nat2); Trinucleotide Repeat Disorders (HTT
(Huntington's Dx), SBMA/SMAX1/AR (Kennedy's Dx), FXN/X25 (Friedrich's Ataxia), ATX3
(Machado-Joseph's Dx), ATXN1 and ATXN2 (spinocerebellar ataxias), DMPK (myotonic dystrophy),
Atrophin-1 and Atn1 (DRPLA Dx), CBP (Creb-BP-global instability), VLDLR (Alzheimer's), Atxn7,
Atxn10).
Occular diseases and Age-related macular degeneration (Abcr, Ccl2, Cc2, cp (ceruloplasmin), disorders
Timp3, cathepsinD, Vidlr, Ccr2); Cataract (CRYAA, CRYA1, CRYBB2, CRYB2, PITX3, BFSP2,
CP49, CP47, CRYAA CRYA1, PAX6, AN2, MGDA, CRYBA1, CRYB1, CRYGC, CRYG3, CCL,
LIM2, MP19, CRYGD, CRYG4, BFSP2, CP49, CP47, HSF4, CTM, HSF4, CTM, MIP, AQP0,
CRYAB, CRYA2, CTPP2, CRYBB1, CRYGD, CRYG4, CRYBB2, CRYB2, CRYGC, CRYG3, CCL,
CRYAA, CRYA1, GJA8, CX50, CAE1, GJA3, CX46, CZP3, CAE3, CCM1, CAM, KRIT1); Corneal
clouding and dystrophy (APOA1, TGFBI, CSD2, CDGG1, CSD, BIGH3, CDG2, TACSTD2, TROP2,
M1S1, VSX1, RINX, PPCD, PPD, KTCN, COL8A2, FECD, PPCD2, PIP5K3, CFD); Cornea plana
congenital (KERA, CNA2); Glaucoma (MYOC, TIGR, GLC1A, JOAG, GPOA, OPTN, GLC1E, FIP2,
HYPL, NRP, CYP1B1, GLC3A, OPA1, NTG, NPG, CYP1B1, GLC3A); Leber congenital amaurosis
(CRB1, RP12, CRX, CORD2, CRD, RPGRIP1, LCA6, CORD9, RPE65, RP20, AIPL1, LCA4,
GUCY2D, GUC2D, LCA1, CORD6, RDH12, LCA3); Macular dystrophy (ELOVL4, ADMD, STGD2,
STGD3, RDS, RP7, PRPH2, PRPH, AVMD, AOFMD, VMD2)

In some aspects, the methods of treating a subject in need thereof comprises administering the bifunctional molecule as provided herein or the composition comprising the bifunctional molecule as provided herein or the pharmaceutical compositions comprising the bifunctional molecule as provided herein to the subject, wherein the administering is effective to treat the subject.

In some embodiments, the subject is a mammal. In some embodiments, the subject is a human.

In some embodiments, the method further comprises administering a second therapeutic agent or a second therapy in combination with the bifunctional molecule as provided herein. In some embodiments, the method comprises administering a first composition comprising the bifunctional molecule as provided herein and a second composition comprising a second therapeutic agent or a second therapy. In some embodiments, the method comprises administering a first pharmaceutical composition comprising the bifunctional molecule as provided herein and a second pharmaceutical composition comprising a second therapeutic agent or a second therapy. In some embodiments, the first composition or the first pharmaceutical composition comprising the bifunctional molecule as provided herein and the second composition or the second pharmaceutical comprising a second therapeutic agent or a second therapy are administered to a subject in need thereof simultaneously, separately, or consecutively.

The terms “treat,” “treating,” and “treatment,” and the like are used herein to generally mean obtaining a desired pharmacological and/or physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly, a human, and includes: (a) preventing the disease from occurring in a subject which may be predisposed to the disease but has not yet been diagnosed as having it; (b) inhibiting the disease, i.e., arresting its development; or (c) relieving the disease, i.e., mitigating or ameliorating the disease and/or its symptoms or conditions. The term “prophylaxis” is used herein to refer to a measure or measures taken for the prevention or partial prevention of a disease or condition.

By “treating or preventing a disease or a condition” is meant ameliorating any of the conditions or signs or symptoms associated with the disorder before or after it has occurred. As compared with an equivalent untreated control, such reduction or degree of prevention is at least 3%, 5%, 10%, 20%, 40%, 50%, 60%, 80%, 90%, 95%, or 100% as measured by any standard technique. A patient who is being treated for a disease or a condition is one who a medical practitioner has diagnosed as having such a disease or a condition. Diagnosis may be by any suitable means. A patient in whom the development of a disease or a condition is being prevented may or may not have received such a diagnosis. One in the art will understand that these patients may have been subjected to the same standard tests as described above or may have been identified, without examination, as one at high risk due to the presence of one or more risk factors (e.g., family history or genetic predisposition).

Diseases and Disorders

In some embodiments, exemplary diseases in a subject to be treated by the bifunctional molecules as provided herein the composition or the pharmaceutical composition comprising the bifunctional molecule as provided herein include, but are not limited to, a cancer, a metabolic disease, an inflammatory disease, a cardiovascular disease, an infectious disease, a genetic disease, a haploinsufficiency disease or a neurological disease.

For instance, examples of cancer, includes, but are not limited to, a malignant, pre-malignant or benign cancer. Cancers to be treated using the disclosed methods include, for example, a solid tumor, a lymphoma or a leukemia. In one embodiment, a cancer can be, for example, a brain tumor (e.g., a malignant, pre-malignant or benign brain tumor such as, for example, a glioblastoma, an astrocytoma, a meningioma, a medulloblastoma or a peripheral neuroectodermal tumor), a carcinoma (e.g., gall bladder carcinoma, bronchial carcinoma, basal cell carcinoma, adenocarcinoma, squamous cell carcinoma, small cell carcinoma, large cell undifferentiated carcinoma, adenomas, cystadenoma, etc.), a basalioma, a teratoma, a retinoblastoma, a choroidea melanoma, a seminoma, a sarcoma (e.g., Ewing sarcoma, rhabdomyosarcoma, craniopharyngeoma, osteosarcoma, chondrosarcoma, myosarcoma, liposarcoma, fibrosarcoma, leimyosarcoma, Askin's tumor, lymphosarcoma, neurosarcoma, Kaposi's sarcoma, dermatofibrosarcoma, angiosarcoma, etc.), a plasmocytoma, a head and neck tumor (e.g., oral, laryngeal, nasopharyngeal, esophageal, etc.), a liver tumor, a kidney tumor, a renal cell tumor, a squamous cell carcinoma, a uterine tumor, a bone tumor, a prostate tumor, a breast tumor including, but not limited to, a breast tumor that is Her2− and/or ER− and/or PR−, a bladder tumor, a pancreatic tumor, an endometrium tumor, a squamous cell carcinoma, a stomach tumor, gliomas, a colorectal tumor, a testicular tumor, a colon tumor, a rectal tumor, an ovarian tumor, a cervical tumor, an eye tumor, a central nervous system tumor (e.g., primary CNS lymphomas, spinal axis tumors, brain stem gliomas, pituitary adenomas, etc.), a thyroid tumor, a lung tumor (e.g., non-small cell lung cancer (NSCLC) or small cell lung cancer), a leukemia or a lymphoma (e.g., cutaneous T-cell lymphomas (CTCL), non-cutaneous peripheral T-cell lymphomas, lymphomas associated with human T-cell lymphotrophic virus (HTLV) such as adult T-cell leukemia/lymphoma (ATLL), B-cell lymphoma, acute non-lymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma, etc.), a multiple myeloma, a skin tumor (e.g., basal cell carcinomas, squamous cell carcinomas, melanomas such as malignant melanomas, cutaneous melanomas or intraocular melanomas, Dermatofibrosarcoma protuberans, Merkel cell carcinoma or Kaposi's sarcoma), a gynecologic tumor (e.g., uterine sarcomas, carcinoma of the fallopian tubes, carcinoma of the endometrium, carcinoma of the cervix, carcinoma of the vagina, carcinoma of the vulva, etc.), Hodgkin's disease, a cancer of the small intestine, a cancer of the endocrine system (e.g., a cancer of the thyroid, parathyroid or adrenal glands, etc.), a mesothelioma, a cancer of the urethra, a cancer of the penis, tumors related to Gorlin's syndrome (e.g., medulloblastomas, meningioma, etc.), a tumor of unknown origin; or metastases of any thereto. In some embodiments, the cancer is a lung tumor, a breast tumor, a colon tumor, a colorectal tumor, a head and neck tumor, a liver tumor, a prostate tumor, a glioma, glioblastoma multiforme, a ovarian tumor or a thyroid tumor; or metastases of any thereto. In some other embodiments, the cancer is an endometrial tumor, bladder tumor, multiple myeloma, melanoma, renal tumor, sarcoma, cervical tumor, leukemia, and neuroblastoma.

For another instance, examples of the metabolic disease include, but are not limited to diabetes, metabolic syndrome, obesity, hyperlipidemia, high cholesterol, arteriosclerosis, hypertension, non-alcoholic steatohepatitis, non-alcoholic fatty liver, non-alcoholic fatty liver disease, hepatic steatosis, and any combination thereof.

For example, the inflammatory disorder partially or fully results from obesity, metabolic syndrome, an immune disorder, an Neoplasm, an infectious disorder, a chemical agent, an inflammatory bowel disorder, reperfusion injury, necrosis, or combinations thereof. In some embodiments, the inflammatory disorder is an autoimmune disorder, an allergy, a leukocyte defect, graft versus host disease, tissue transplant rejection, or combinations thereof. In some embodiments, the inflammatory disorder is a bacterial infection, a protozoal infection, a protozoal infection, a viral infection, a fungal infection, or combinations thereof. In some embodiments, the inflammatory disorder is Acute disseminated encephalomyelitis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barré syndrome; Hashimoto's disease; Idiopathic thrombocytopenic purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjögren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behçet's syndrome; Infective colitis; Indeterminate colitis; Inflammatory liver disorder, Endotoxin shock, Rheumatoid spondylitis, Ankylosing spondylitis, Gouty arthritis, Polymyalgia rheumatica, Alzheimer's disorder, Parkinson's disorder, Epilepsy, AIDS dementia, Asthma, Adult respiratory distress syndrome, Bronchitis, Cystic fibrosis, Acute leukocyte-mediated lung injury, Distal proctitis, Wegener's granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Hyperacute transplant rejection, Acute transplant rejection, Chronic transplant rejection, Acute graft-versus-host disease, Chronic graft-versus-host disease, abdominal aortic aneurysm (AAA); or combinations thereof.

For another instance, examples of the neurological disease include, but are not limited to, Aarskog syndrome, Alzheimer's disease, amyotrophic lateral sclerosis (Lou Gehrig's disease), aphasia, Bell's Palsy, Creutzfeldt-Jakob disease, cerebrovascular disease, Cornelia de Lange syndrome, epilepsy and other severe seizure disorders, dentatorubral-pallidoluysian atrophy, fragile X syndrome, hypomelanosis of Ito, Joubert syndrome, Kennedy's disease, Machado-Joseph's diseases, migraines, Moebius syndrome, myotonic dystrophy, neuromuscular disorders, Guillain-Barre, muscular dystrophy, neuro-oncology disorders, neurofibromatosis, neuro-immunological disorders, multiple sclerosis, pain, pediatric neurology, autism, dyslexia, neuro-otology disorders, Meniere's disease, Parkinson's disease and movement disorders, Phenylketonuria, Rubinstein-Taybi syndrome, sleep disorders, spinocerebellar ataxia I, Smith-Lemli-Opitz syndrome, Sotos syndrome, spinal bulbar atrophy, type 1 dominant cerebellar ataxia, Tourette syndrome, tuberous sclerosis complex and William's syndrome.

The term “cardiovascular disease,” as used herein, refers to a disorder of the heart and blood vessels, and includes disorders of the arteries, veins, arterioles, venules, and capillaries. Non-limiting examples of cardiovascular diseases include coronary artery diseases, cerebral strokes (cerebrovascular disorders), peripheral vascular diseases, myocardial infarction and angina, cerebral infarction, cerebral hemorrhage, cardiac hypertrophy, arteriosclerosis, and heart failure.

The term “infectious disease,” as used herein, refer to any disorder caused by organisms, such as prions, bacteria, viruses, fungi and parasites. Examples of an infectious disease include, but are not limited to, strep throat, urinary tract infections or tuberculosis caused by bacteria, the common cold, measles, chickenpox, or AIDS caused by viruses, skin diseases, such as ringworm and athlete's foot, lung infection or nervous system infection caused by fungi, and malaria caused by a parasite. Examples of viruses that can cause an infectious disease include, but are not limited to, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Coronavirus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, Norovirus, O'nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Severe acute respiratory syndrome coronavirus 2, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus. Examples of infectious diseases caused by parasites include, but are not limited to, Acanthamoeba Infection, Acanthamoeba Keratitis Infection, African Sleeping Sickness (African trypanosomiasis), Alveolar Echinococcosis (Echinococcosis, Hydatid Disease), Amebiasis (Entamoeba histolytica Infection), American Trypanosomiasis (Chagas Disease), Ancylostomiasis (Hookworm), Angiostrongyliasis (Angiostrongylus Infection), Anisakiasis (Anisakis Infection, Pseudoterranova Infection), Ascariasis (Ascaris Infection, Intestinal Roundworms), Babesiosis (Babesia Infection), Balantidiasis (Balantidium Infection), Balamuthia, Baylisascariasis (Baylisascaris Infection, Raccoon Roundworm), Bed Bugs, Bilharzia (Schistosomiasis), Blastocystis hominis Infection, Body Lice Infestation (Pediculosis), Capillariasis (Capillaria Infection), Cercarial Dermatitis (Swimmer's Itch), Chagas Disease (American Trypanosomiasis), Chilomastix mesnili Infection (Nonpathogenic [Harmless] Intestinal Protozoa), Clonorchiasis (Clonorchis Infection), CLM (Cutaneous Larva Migrans, Ancylostomiasis, Hookworm), “Crabs” (Pubic Lice), Cryptosporidiosis (Cryptosporidium Infection), Cutaneous Larva Migrans (CLM, Ancylostomiasis, Hookworm), Cyclosporiasis (Cyclospora Infection), Cysticercosis (Neurocysticercosis), Cystoisospora Infection (Cystoisosporiasis) formerly Isospora Infection, Dientamoeba fragilis Infection, Diphyllobothriasis (Diphyllobothrium Infection), Dipylidium caninum Infection (dog or cat tapeworm infection), Dirofilariasis (Dirofilaria Infection), DPDx, Dracunculiasis (Guinea Worm Disease), Dog tapeworm (Dipylidium caninum Infection), Echinococcosis (Cystic, Alveolar Hydatid Disease), Elephantiasis (Filariasis, Lymphatic Filariasis), Endolimax nana Infection (Nonpathogenic [Harmless] Intestinal Protozoa), Entamoeba coli Infection (Nonpathogenic [Harmless] Intestinal Protozoa), Entamoeba dispar Infection (Nonpathogenic [Harmless] Intestinal Protozoa), Entamoeba hartmanni Infection (Nonpathogenic [Harmless] Intestinal Protozoa), Entamoeba histolytica Infection (Amebiasis), Entamoeba polecki, Enterobiasis (Pinworm Infection), Fascioliasis (Fasciola Infection), Fasciolopsiasis (Fasciolopsis Infection), Filariasis (Lymphatic Filariasis, Elephantiasis), Giardiasis (Giardia Infection), Gnathostomiasis (Gnathostoma Infection), Guinea Worm Disease (Dracunculiasis), Head Lice Infestation (Pediculosis), Heterophyiasis (Heterophyes Infection), Hookworm Infection, Human, Hookworm Infection, Zoonotic (Ancylostomiasis, Cutaneous Larva Migrans [CLM]), Hydatid Disease (Cystic, Alveolar Echinococcosis), Hymenolepiasis (Hymenolepis Infection), Intestinal Roundworms (Ascariasis, Ascaris Infection), Iodamoeba buetschlii Infection (Nonpathogenic [Harmless] Intestinal Protozoa), Isospora Infection (see Cystoisospora Infection), Kala-azar (Leishmaniasis, Leishmania Infection), Keratitis (Acanthamoeba Infection), Leishmaniasis (Kala-azar, Leishmania Infection), Lice Infestation (Body, Head, or Pubic Lice, Pediculosis, Pthiriasis), Liver Flukes (Clonorchiasis, Opisthorchiasis, Fascioliasis), Loiasis (Loa loa Infection), Lymphatic filariasis (Filariasis, Elephantiasis), Malaria (Plasmodium Infection), Microsporidiosis (Microsporidia Infection), Mite Infestation (Scabies), Myiasis, Naegleria Infection, Neurocysticercosis (Cysticercosis), Ocular Larva Migrans (Toxocariasis, Toxocara Infection, Visceral Larva Migrans), Onchocerciasis (River Blindness), Opisthorchiasis (Opisthorchis Infection), Paragonimiasis (Paragonimus Infection), Pediculosis (Head or Body Lice Infestation), Pthiriasis (Pubic Lice Infestation), Pinworm Infection (Enterobiasis), Plasmodium Infection (Malaria), Pneumocystis jirovecii Pneumonia, Pseudoterranova Infection (Anisakiasis, Anisakis Infection), Pubic Lice Infestation (“Crabs,” Pthiriasis), Raccoon Roundworm Infection (Baylisascariasis, Baylisascaris Infection), River Blindness (Onchocerciasis), Sappinia, Sarcocystosis (Sarcocystosis Infection), Scabies, Schistosomiasis (Bilharzia), Sleeping Sickness (Trypanosomiasis, African; African Sleeping Sickness), Soil-transmitted Helminths, Strongyloidiasis (Strongyloides Infection), Swimmer's Itch (Cercarial Dermatitis), Taeniasis (Taenia Infection, Tapeworm Infection), Tapeworm Infection (Taeniasis, Taenia Infection), Toxocariasis (Toxocara Infection, Ocular Larva Migrans, Visceral Larva Migrans), Toxoplasmosis (Toxoplasma Infection), Trichinellosis (Trichinosis), Trichinosis (Trichinellosis), Trichomoniasis (Trichomonas Infection), Trichuriasis (Whipworm Infection, Trichuris Infection), Trypanosomiasis, African (African Sleeping Sickness, Sleeping Sickness), Trypanosomiasis, American (Chagas Disease), Visceral Larva Migrans (Toxocariasis, Toxocara Infection, Ocular Larva Migrans), Whipworm Infection (Trichuriasis, Trichuris Infection), Zoonotic Diseases (Diseases spread from animals to people), and Zoonotic Hookworm Infection (Ancylostomiasis, Cutaneous Larva Migrans [CLM]). Examples of infectious diseases caused by fungi include, but are not limited to, Apergillosis, Balsomycosis, Candidiasis, Cadidia auris, Coccidioidomycosis, C. neoformans infection, C gattii infection, fungal eye infections, fungal nail infections, histoplasmosis, mucormycosis, mycetoma, Pneuomcystis pneumonia, ringworm, sporotrichosis, cyrpococcosis, and Talaromycosis. Examples of bacteria that can cause an infectious disease include, but are not limited to, Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeium and Corynebacterium), Clostridium sp. (such as Clostridium perfringens, Clostridium dificile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Microsporum canis, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Pityrosporum orbiculare (Malassezia furfur), Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Trichophyton rubrum, T. mentagrophytes, Tropheryma whippeihi, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia.

The term “genetic disease,” as used herein, refers to a health problem caused by one or more abnormalities in the genome. It can be caused by a mutation in a single gene (monogenic) or multiple genes (polygenic) or by a chromosomal abnormality. The single gene disease may be related to an autosomal dominant, autosomal recessive, X-linked dominant, X-linked recessive, Y-linked, or mitochondrial mutation. Examples of genetic diseases include, but are not limited to, lp36 deletion syndrome, 18p deletion syndrome, 21-hydroxylase deficiency, 47,XXX (triple X syndrome), AAA syndrome (achalasia-addisonianism-alacrima syndrome), Aarskog-Scott syndrome, ABCD syndrome, Aceruloplasminemia, Acheiropodia, Achondrogenesis type II, achondroplasia, Acute intermittent porphyria, adenylosuccinate lyase deficiency, Adrenoleukodystrophy, ADULT syndrome, Aicardi-Goutieres syndrome, Alagille syndrome, Albinism, Alexander disease, alkaptonuria, Alpha 1-antitrypsin deficiency, Alport syndrome, Alstrom syndrome, Alternating hemiplegia of childhood, Alzheimer's disease, Amelogenesis imperfecta, Aminolevulinic acid dehydratase deficiency porphyria, Amyotrophic lateral sclerosis—Frontotemporal dementia, Androgen insensitivity syndrome, Angelman syndrome, Apert syndrome, Arthrogryposis-renal dysfunction-cholestasis syndrome, Ataxia telangiectasia, Axenfeld syndrome, Beare-Stevenson cutis gyrata syndrome, Beckwith-Wiedemann syndrome, Benjamin syndrome, biotinidase deficiency, Birt-Hogg-Dube syndrome, Bjornstad syndrome, Bloom syndrome, Brody myopathy, Brunner syndrome, CADASIL syndrome, Campomelic dysplasia, Canavan disease, CARASIL syndrome, Carpenter Syndrome, Cerebral dysgenesis-neuropathy-ichthyosis-keratoderma syndrome (SEDNIK), Charcot-Marie-Tooth disease, CHARGE syndrome, Chediak-Higashi syndrome, Chronic granulomatous disorder, Cleidocranial dysostosis, Cockayne syndrome, Coffin-Lowry syndrome, Cohen syndrome, collagenopathy, types II and XI, Congenital insensitivity to pain with anhidrosis (CIPA), Congenital Muscular Dystrophy, Cornelia de Lange syndrome (CDLS), Cowden syndrome, CPO deficiency (coproporphyria), Cranio-lenticulo-sutural dysplasia, Cri du chat, Crohn's disease, Crouzon syndrome, Crouzonodermoskeletal syndrome (Crouzon syndrome with acanthosis nigricans), Cystic fibrosis, Darier's disease, De Grouchy syndrome, Dent's disease (Genetic hypercalciuria), Denys-Drash syndrome, Di George's syndrome, Distal hereditary motor neuropathies, multiple types, Distal muscular dystrophy, Down Syndrome, Dravet syndrome, Duchenne muscular dystrophy, Edwards Syndrome, Ehlers-Danlos syndrome, Emery-Dreifuss syndrome, Epidermolysis bullosa, Erythropoietic protoporphyria, Fabry disease, Factor V Leiden thrombophilia, Familial adenomatous polyposis, Familial Creutzfeld-Jakob Disease, Familial dysautonomia, Fanconi anemia (FA), Fatal familial insomnia, Feingold syndrome, FG syndrome, Fragile X syndrome, Friedreich's ataxia, G6PD deficiency, Galactosemia, Gaucher disease, Gerstmann-Straussler-Scheinker syndrome, Gillespie syndrome, Glutaric aciduria, type I and type 2, GRACILE syndrome, Griscelli syndrome, Hailey-Hailey disease, Harlequin type ichthyosis, Hemochromatosis, hereditary, Hemophilia, Hepatoerythropoietic porphyria, Hereditary coproporphyria, Hereditary hemorrhagic telangiectasia (Osler-Weber-Rendu syndrome), Hereditary inclusion body myopathy, Hereditary multiple exostoses, Hereditary neuropathy with liability to pressure palsies (HNPP), Hereditary spastic paraplegia (infantile-onset ascending hereditary spastic paralysis), Hermansky-Pudlak syndrome, Heterotaxy, Homocystinuria, Hunter syndrome, Huntington's disease, Hurler syndrome, Hutchinson-Gilford progeria syndrome, Hyperlysinemia, Hyperoxaluria, Hyperphenylalaninemia, Hypoalphalipoproteinemia (Tangier disease), Hypochondrogenesis, Hypochondroplasia, Immunodeficiency-centromeric instability-facial anomalies syndrome (ICF syndrome), Incontinentia pigmenti, Ischiopatellar dysplasia, Isodicentric 15, Jackson-Weiss syndrome, Joubert syndrome, Juvenile primary lateral sclerosis (JPLS), Keloid disorder, Kniest dysplasia, Kosaki overgrowth syndrome, Krabbe disease, Kufor-Rakeb syndrome, LCAT deficiency, Lesch-Nyhan syndrome, L₁-Fraumeni syndrome, Limb-Girdle Muscular Dystrophy, lipoprotein lipase deficiency, Lynch syndrome, Malignant hyperthermia, Maple syrup urine disease, Marfan syndrome, Maroteaux-Lamy syndrome, McCune-Albright syndrome, McLeod syndrome, Mediterranean fever, familial, MEDNIK syndrome, Menkes disease, Methemoglobinemia, Methylmalonic acidemia, Micro syndrome, Microcephaly, Morquio syndrome, Mowat-Wilson syndrome, Muenke syndrome, Multiple endocrine neoplasia type 1 (Wermer's syndrome), Multiple endocrine neoplasia type 2, Muscular dystrophy, Muscular dystrophy, Duchenne and Becker type, Myostatin-related muscle hypertrophy, myotonic dystrophy, Natowicz syndrome, Neurofibromatosis type I, Neurofibromatosis type II, Niemann-Pick disease, Nonketotic hyperglycinemia, Nonsyndromic deafness, Noonan syndrome, Norman-Roberts syndrome, Ogden syndrome, Omenn syndrome, Osteogenesis imperfecta, Pantothenate kinase-associated neurodegeneration, Patau syndrome (Trisomy 13), PCC deficiency (propionic acidemia), Pendred syndrome, Peutz-Jeghers syndrome, Pfeiffer syndrome, Phenylketonuria, Pipecolic acidemia, Pitt-Hopkins syndrome, Polycystic kidney disease, Polycystic ovary syndrome (PCOS), Porphyria, Porphyria cutanea tarda (PCT), Prader-Willi syndrome, Primary ciliary dyskinesia (PCD), Primary pulmonary hypertension, Protein C deficiency, Protein S deficiency, Pseudo-Gaucher disease, Pseudoxanthoma elasticum, Retinitis pigmentosa, Rett syndrome, Roberts syndrome, Rubinstein-Taybi syndrome (RSTS), Sandhoff disease, Sanfilippo syndrome, Schwartz-Jampel syndrome, Shprintzen-Goldberg syndrome, Sickle cell anemia, Siderius X-linked mental retardation syndrome, Sideroblastic anemia, Sjogren-Larsson syndrome, Sly syndrome, Smith-Lemli-Opitz syndrome, Smith-Magenis syndrome, Snyder-Robinson syndrome, Spinal muscular atrophy, Spinocerebellar ataxia (types 1-29), Spondyloepiphyseal dysplasia congenita (SED), SSB syndrome (SADDAN), Stargardt disease (macular degeneration), Stickler syndrome (multiple forms), Strudwick syndrome (spondyloepimetaphyseal dysplasia, Strudwick type), Tay-Sachs disease, Tetrahydrobiopterin deficiency, Thanatophoric dysplasia, Treacher Collins syndrome, Tuberous sclerosis complex (TSC), Turner syndrome, Usher syndrome, Variegate porphyria, von Hippel-Lindau disease, Waardenburg syndrome, Weissenbacher-Zweymuller syndrome, Williams syndrome, Wilson disease, Wolf-Hirschhorn syndrome, Woodhouse-Sakati syndrome, X-linked intellectual disability and macroorchidism (fragile X syndrome), X-linked severe combined immunodeficiency (X-SCID), X-linked sideroblastic anemia (XLSA), X-linked spinal-bulbar muscle atrophy (spinal and bulbar muscular atrophy), Xeroderma pigmentosum, Xp11.2 duplication syndrome, XXXX syndrome (48, XXXX), XXXXX syndrome (49, XXXXX), XYY syndrome (47,XYY), Zellweger syndrome.

All references, publications, patents, and patent applications mentioned in this specification are herein incorporated by reference to the same extent as if each individual publication, patent, or patent application was specifically and individually indicated to be incorporated by reference.

The above described embodiments can be combined to achieve the afore-mentioned functional characteristics. This is also illustrated by the below examples which set forth exemplary combinations and functional characteristics achieved.

### EXAMPLES

The following examples are provided to further illustrate some embodiments of the present disclosure, but are not intended to limit the scope of the present disclosure; it will be understood by their exemplary nature that other procedures, methodologies, or techniques known to those skilled in the art may alternatively be used.

### Example 1: Generating Binding ASOs to RNA Targets

Methods to design antisense oligonucleotides to RNA transcripts encoding Renilla luciferase (Rluc) were developed and tested.

The sequence of Rluc (Genbank accession number: AF025846) was run on a publicly-available program (//ma.tbi.univie.ac.at/cgi-bin/RNAxs/RNAxs.cgi) to identify regions suitable for high binding energy ASOs, typically lower than −8 kcal, using 20 nucleotides as sequence length. ASOs with more than 3 consecutive G nucleotides were excluded. The ASOs with the highest binding energy were then processed through BLAST (NCBI) to check their potential binding selectivity based on nucleotide sequence, and those with at least 2 mismatches to other sequences were retained. The selected ASOs were then synthesized as described below.

5′-Amino ASO Synthesis

5′-Amino ASO was synthesized with a typical step-wise solid phase oligonucleotide synthesis method on a Dr. Oligo 48 (Biolytic Lab Performance Inc.) synthesizer, according to manufacturer's protocol. A 1000 nmol scale universal CPG column (Biolytic Lab Performance Inc. part number 168-108442-500) was utilized as the solid support. The monomers were modified RNA phosphoramidites with protecting groups (5′-O-(4,4′-Dimethoxytrityl)-2′-O-methoxyethyl-N6-benzoyl-adenosine-3′-O-[(2-cyanoethyl)-(N,N-diisopropyl)]-phosphoramidite, 5′-O-(4,4′-Dimethoxytrityl)-2′-O-methoxyethyl-5-methyl-N4-benzoyl-cytidine-3′-O-[(2-cyanoethyl)-(N,N-diisopropyl)]-phosphoramidite, 5′-O-(4,4′-Dimethoxytrityl)-2′-O-methoxyethyl-N2-isobutyryl-guanosine-3′-O-[(2-cyanoethyl)-(N,N-diisopropyl)]-phosphoramidite, 5′-O-(4,4′-Dimethoxytrityl)-2′-O-methoxyethyl-5-methyl-uridine-3′-O-[(2-cyanoethyl)-(N,N-diisopropyl)]-phosphoramidite) purchased from Chemgenes Corporation. The 5′-amino modification required the use of the TFA-amino C6-CED phosphoramidite (6-(Trifluoroacetylamino)-hexyl-(2-cyanoethyl)-(N, N-diisopropyl)-phosphoramidite) in the last step of synthesis. All monomers were diluted to 0.1M with anhydrous acetonitrile (Fisher Scientific BP 1170) prior to being used on the synthesizer.

The commercial reagents used for synthesis on the oligonucleotide synthesizer, including 3% trichloroacetic acid in dichloromethane (DMT removal reagent, RN-1462), 0.3M benzylthiotetrazole in acetonitrile (activation reagent, RN-1452), 0.1M ((Dimethylamino-methylidene)amino)-3H-1,2,4-dithiazoline-3-thione in 9:1 pyridine/acetonitrile (sulfurizing reagent, RN-1689), 0.2M iodine/pyridine/water/tetrahydrofuran (oxidation solution, RN-1455), acetic anhydride/pyridine/tetrahydrofuran (CAP A solution, RN-1458), 10% N-methylimidazole in tetrahydrofuran (CAP B solution, RN-1481), were purchased from ChemGenes Corporation. Anhydrous acetonitrile (wash reagent, BP 1170) was purchased from Fisher Scientific for use on the synthesizer. All solutions and reagents were kept anhydrous with the use of drying traps (DMT-1975, DMT-1974, DMT-1973, DMT-1972) purchased from ChemGenes Corporation.

Cyanoethyl Protecting Group Removal

In order to prevent acrylonitrile adduct formation on the primary amine, the 2′-cyanoethyl protecting groups were removed prior to deprotection of the amine. A solution of 10% diethylamine in acetonitrile was added to column as needed to maintain contact with the column for 5 minutes. The column was then washed 5 times with 500 uL of acetonitrile.

Deprotection and Cleavage

The oligonucleotide was cleaved from the support with simultaneous deprotection of other protecting groups. The column was transferred to a screw cap vial with a pressure relief cap (ChemGlass Life Sciences CG-4912-01). 1 mL of ammonium hydroxide was added to the vial and the vial was heated to 55° C. for 16 hours. The vial was cooled to room temperature and the ammonia solution was transferred to a 1.5 mL microfuge tube. The CPG support was washed with 200 uL of RNAse free molecular biology grade water and the water was added to the ammonia solution. The resulting solution was concentrated in a centrifugal evaporator (SpeedVac SPD1030).

Precipitation

The residue was dissolved in 360 uL of RNAse free molecular biology grade water and 40 uL of a 3M sodium acetate buffer solution was added. To remove impurities, the microfuge tube was centrifuged at a high speed (14000 g) for 10 minutes. The supernatant was transferred to a tared 2 mL microfuge tube. 1.5 mL of ethanol was added to the clear solution and tube was vortexed and then stored at −20° C. for 1 hour. The microfuge tube was then centrifuged at a high speed (14000 g) at 5° C. for 15 minutes. The supernatant was carefully removed, without disrupting the pellet, and the pellet was dried in the SpeedVac. The oligonucleotide yield was estimated by mass calculation and the pellet was resuspended in RNAse free molecular biology grade water to give an 8 mM solution which was used in subsequent steps.

It was demonstrated that ASOs targeting a specific RNA target shown in Tables 1A and 1B were designed and synthesized successfully.

### Example 2: Design and Synthesis of the Bifunctional Molecule

Methods to conjugate ASOs targeting Rule to a small molecule were developed and tested. To target Rluc, a bifunctional modality was used. The modality included two domains, a first domain that targets a specific RNA molecule (this domain can be an RNA binding protein, an ASO, or a small molecule) and a second domain (e.g., a protein, aptamer, small molecule/inhibitor) that interacts with a protein that modulates the translation of the targeted RNA, with the two domains connected by a linker. The modality was Renilla Luciferase (Rluc) targeting ASOs linked to a small molecule, Ibrutinib or Ibrutinib-MPEA, which binds/recruits the ATP-binding pocket of Bruton's Tyrosine Kinase (BTK) protein (//doi.org/10.1124/mol.116.107037).

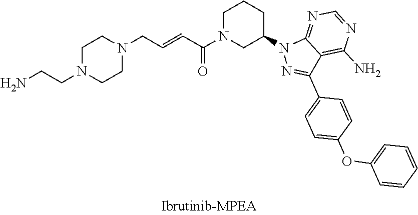 [SMILES: NCCN1CCN(C/C=C/C(=O)N2CCC[C@@H](n3nc(-c4ccc(Oc5ccccc5)cc4)c4c(N)ncnc43)C2)CC1]

The synthesized 5′-amino ASOs from Example 1 were used to make ASO-small molecule conjugates following scheme 1 below.

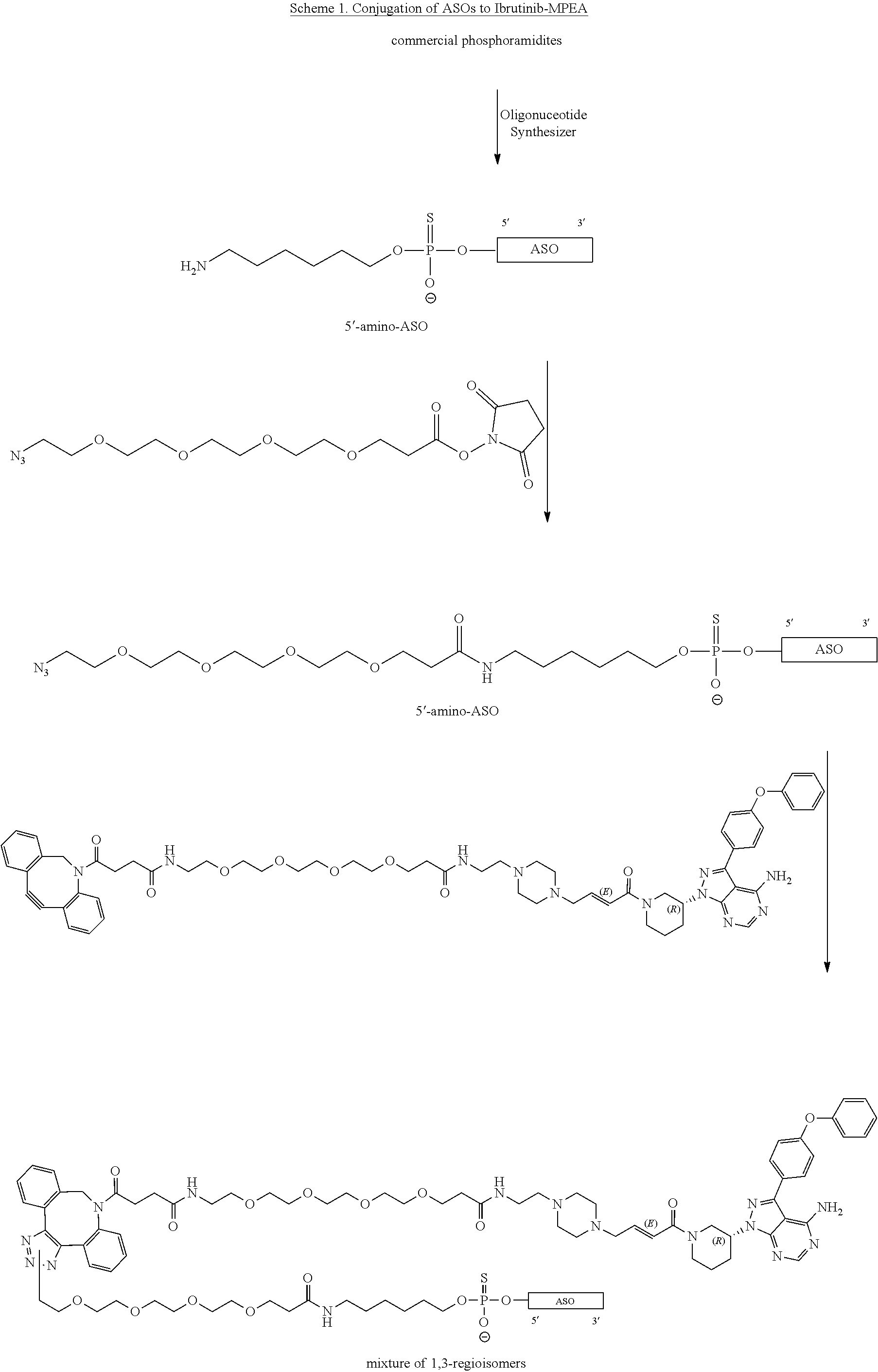

5′-azido-ASO was generated from 5′-amino-ASO.

A solution of 5′-amino ASO (2 mM, 15 μL, 30 nmole) was mixed with a sodium borate buffer (pH 8.5, 75 μL). A solution of N3-PEG4-NHS ester (10 mM in DMSO, 30 μL, 300 nmol) was then added, the mixture was orbitally shaken at room temperature for 16 hours. The solution was dried overnight with a SpeedVac. The resulting residue was redissolved in water (20 μL) and purified by RP-HPLC reverse phase to provide 5′-azido ASO (12-21 nmol by nanodrop UV-VIS quantitation). This 5′-azido ASO solution in water (2 mM in water, 7 μL) was mixed with Ibrutinib-MPEA-PEG4-DBCO (synthesized from DBCO-PEG4-NHS and Ibrutinib-MPEA and purified by reverse phase HPLC, 2 mM in DMSO, 21 μL) in a PCR tube and was orbitally shaken at room temperature for 16 hours. The reaction mixture was dried at room temperature for 6-16 hours with SpeedVac. The resulting residue was redissolved in water (20 μL), centrifuged to provide clear supernatant, which was transferred, purified by reverse phase HPLC to provide ASO-Linker-Ibrutinib-MPEA conjugate as a mixture of 1,3-regioisomers (4.0-7.8 nmol by nanodrop UV-VIS quantitation). In some cases, the reaction mixture was directly injected into HPLC for purification. The conjugate was characterized and confirmed by matrix-assisted laser desorption/ionization time of flight mass spectrometry (MALDI-TOF MS) or electrospray ionization mass spectrometry (ESI-MS). Exemplary result is shown in FIG. 1.

### Example 3: Formation of RNA-Bifunctional-Protein Ternary Complex In Vitro

Methods to form an RNA-bifunctional-protein ternary complex were developed and tested.

### Example 3a: Bifunctional Design

The bifunctional molecules are composed of ASOs, linker, and Ibrutinib-MPEA. ASOs are the RNA binder part of the bifunctional molecules. Ibrutinib-MPEA is the effector/protein recruiter. ASOs and Ibrutinib-MPEA are hooked together by a linker as shown in Scheme 1. Inhibitor Ibrutinib that covalently binds to the ATP-binding pocket of Bruton's Tyrosine Kinase (BTK) protein (//doi.org/10.1124/mol.116.107037) was conjugated to ASOs. To generate the conjugate, the protocols in Examples 1 and 2 were followed.

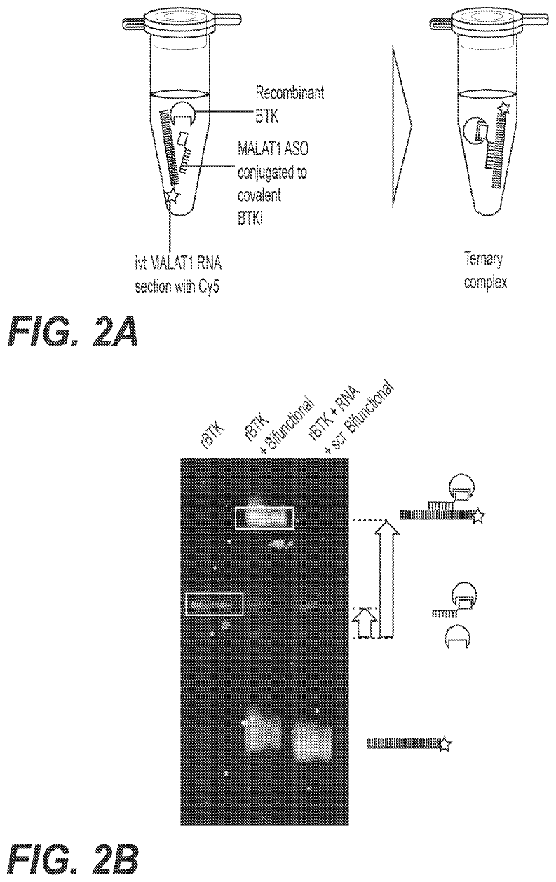

A ternary complex is a complex containing three different molecules bound together. A complex of the bifunctional molecule was demonstrated to interact with its target RNA and its target protein by its ASO and small molecule domains, respectively. An inhibitor-conjugated antisense oligonucleotide (hereafter referred to as ASOi) (i.e., Rluc ASO conjugated to Ibrutinib-MPEA) was mixed with the protein target of the inhibitor (i.e., BTK) and the RNA target of the ASO (i.e., Rluc RNA), and allowed to react with the protein and hybridize with the RNA target to form a ternary complex including all 3 molecules. The same was also performed with MALAT1 targeting ASO with the sequence 5′CGUUAACUAGGCUUUA3′ (SEQ ID NO: 4) conjugated at the 5′ end with Ibrutinib (BTK inhibitor; BTKi) and the RNA target of the ASO (i.e., MALAT1 RNA) as shown in FIG. 2A. FIG. 2B depicts the gel analysis results detecting the formation of the ternary complex. Binding of the ASOi to the target protein caused the protein to migrate higher (shift up) on a polyacrylamide gel because of its increased molecular weight. Additional hybridization of the target RNA to the ASOi-protein complex “supershifted” the protein band even higher on the gel, indicating that all 3 components were stably associated in the complex. Furthermore, labeling the target RNA with a fluorescent dye allowed direct visualization of the target RNA in the supershifted protein complex.

### Example 3b: In Vitro Ternary Complex Formation Assay

In one reaction (#1), 10 pmol of the MALAT1 targeting ASO (hereafter called N33-ASOi) conjugated at the 5′ end with Ibrutinib was mixed in PBS with 2 pmol purified BTK protein, 200 pmol yeast rRNA (as non-specific blocker) and 20 pmol Cy5-labeled IVT RNA of the following sequence:

(SEQ ID NO: 5)
5’CCUUGAAAUCCAUGACGCAGGGAGAAUUGCGUCAUUUAAAGCCUAGU
UAACGCAUUUACUAAACGCAGACGAAAAUGGAAAGAUUAAUUGGGAGUG
GUAGGAUGAAACAAUUUGGAGAAGAUAGAAGUUUGAAGUGGAAAACUGG
AAGACAGAAGUACGGGAAGGCGAA3’.

As controls, the following reactions were mixed in PBS with 200 pmol yeast tRNA and the following components:
(#2) 2 pmol purified BTK protein only (to identify band size on gel of non-complexed protein);
(#3) 2 pmol purified BTK protein and 10 pmol N33-ASOi (to identify size of 2-component shifted band);
(#4) 2 pmol purified BTK protein and 20 pmol Cy5-IVT RNA above (to test whether the target RNA interacts directly with the Cy5-IVT RNA);
(#5) 10 pmol non-complementary RNA oligo of the sequence 5′AGAGGUGGCGUGGUAG3′ (SEQ ID NO: 6; hereafter called SCR-ASOi) conjugated at the 5′ end with Ibrutinib and 2 pmol purified BTK protein (to test whether the formation of the ternary complex requires a complementary ASO sequence); and
(#6) 2 pmol purified BTK protein and 10 pmol SCR-ASOi (to show that the Ibrutinib-modified scrambled ASO is capable of size-shifting the BTK protein band).

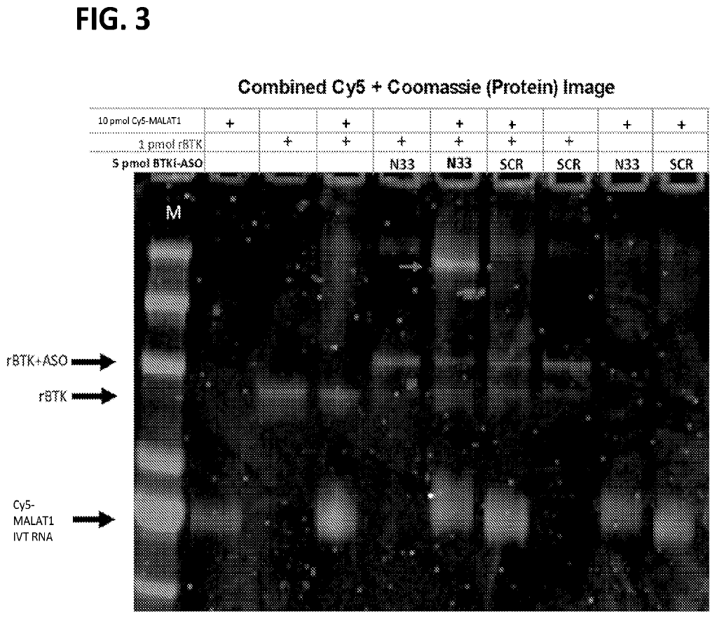

All reactions were incubated at room temperature for 90 minutes protected from light, then mixed with a loading buffer containing 0.5% SDS final and 10% glycerol final, and complexes separated by PAGE on a Bis-Tris 4-12% gel including an IRDye700 pre-stained protein molecular weight marker (LiCor). Immediately following electrophoresis, the gel was imaged using a LiCor Odyssey system with the 700 nm channel to identify the position of Cy5-IVT-RNA bands and MW marker. Next, proteins in the gel were stained using InstantBlue colloidal Coomassie stain (Expedeon) and re-imaged using transmitted light. The two images were lined up using size markers and lane positions to identify the relative positions of BTK protein bands and Cy5-IVT target RNA. (FIG. 3)

An increase in MW of the BTK protein band when reacted with N33-ASOi (samples 1 and 3 vs. 2) indicated binary complex formation, and a further supershift in the presence of Cy5-IVT RNA (Sample 1 vs. sample 3) observed with N33-ASOi but not with SCR-ASOi demonstrated that all 3 components were present in the complex and that formation is specific to hybridizing a complementary sequence. This complex was further confirmed by Cy5-IVT-RNA fluorescence signal overlapping the super-shifted BTK protein band.

A bifunctional molecule was observed to interact with the target RNA via the ASO and the target protein by the small molecule.

### Example 4: Increasing RNA Translation Bifunctional Molecules and BTK-Fused Effectors

Methods to enhance the translation of a target RNAs by an effector protein and a bifunctional molecule were developed and tested.

### Example 4a; Bifunctional Design

Each of ASO2 and ASO3 targeting the mRNA encoding Renilla Luciferase protein was conjugated at the 5′ end with Ibrutinib-MPEA as described in Example 2a. A non-targeting control ASO1 was also conjugated at the 5′ end with Ibrutinib-MPEA as described in example 3a.

### Example 4b: Target Vector Design

The target transcript encoding Renilla luciferase mRNA and protein were expressed from the pRL-TK vector (Promega Corp., Genbank accession number: AF025846).

### Example 4c: Effector Vector Design

A mammalian expression plasmid was generated by synthesizing and cloning a cytomegalovirus (CMV) enhancer and promoter and a polyadenylation signal (DNA fragments synthesized by Integrated DNA Technologies). The DNA sequence encoding the effector was synthesized (Integrated DNA Technologies) and subsequently cloned between the CMV promoter and the polyA signal. The effector was made of the following parts in N-terminus to C-terminus order:

A sequence encoding the BTK protein, with the following amino acid sequence:

(SEQ ID NO: 7)
KNAPSTAGLGYGSWEIDPKDLTFLKELGTGQFGVVKYGKWRGQYDVAIK
MIKEGSMSEDEFIEEAKVMMNLSHEKLVQLYGVCTKQRPIFIITEYMAN
GCLLNYLREMRHRFQTQQLLEMCKDVCEAMEYLESKQFLHRDLAARNCL
VNDQGVVKVSDFGLSRYVLDDEYTSSVGSKFPVRWSPPEVLMYSKFSSK
SDIWAFGVLMWEIYSLGKMPYERFTNSETAEHIAQGLRLYRPHLASEKV
YTIMYSCWHEKADERPTFKILLSNILDVMDEES

A sequence encoding the EIF4E protein, with the following amino acid sequence:

(SEQ ID NO: 8)
MATVEPETTPTPNPPTTEEEKTESNQEVANPEHYIKHPLQNRWALWFFK
NDKSKTWQANLRLISKFDTVEDFWALYNHIQLSSNLMPGCDYSLFKDGI
EPMWEDEKNKRGGRWLITLNKQQRRSDLDRFWLETLLCLIGESFDDYSD
DVCGAVVNVRAKGDKIAIWTTECENREAVTHIGRVYKERLGLPPKIVIG
YQSHADTATKSGSTTKNRFVV

A sequence encoding the YTHDF1 protein, with the following amino acid sequence:

(SEQ ID NO: 9)
MSATSVDTQRTKGQDNKVQNGSLHQKDTVHDNDFEPYLTGQSNQSNSYP
SMSDPYLSSYYPPSIGFPYSLNEAPWSTAGDPPIPYLTTYGQLSNGDHH
FMHDAVFGQPGGLGNNIYQHRFNFFPENPAFSAWGTSGSQGQQTQSSAY
GSSYTYPPSSLGGTVVDGQPGFHSDTLSKAPGMNSLEQGMVGLKIGDVS
SSAVKTVGSVVSSVALTGVLSGNGGTNVNMPVSKPTSWAAIASKPAKPQ
PKMKTKSGPVMGGGLPPPPIKHNMDIGTWDNKGPVPKAPVPQQAPSPQA
APQPQQVAQPLPAQPPALAQPQYQSPQQPPQTRWVAPRNRNAAFGQSGG
AGSDSNSPGNVQPNSAPSVESHPVLEKLKAAHSYNPKEFEWNLKSGRVF
IIKSYSEDDIHRSIKYSIWCSTEHGNKRLDSAFRCMSSKGPVYLLFSVN
GSGHFCGVAEMKSPVDYGTSAGVWSQDKWKGKFDVQWIFVKDVPNNQLR
HIRLENNDNKPVTNSRDTQEVPLEKAKQVLKIISSYKHTTSIFDDFAHY
EKRQEEEEVVRKERQSRNKQ

A sequencing encoding the T₂A self-cleaving peptide, with the following amino acid sequence:

(SEQ ID NO: 10)
EGRGSLLTCGDVEENPGP

A sequence encoding a monomeric enhanced fluorescence protein (mEGFP) protein, with the following amino acid sequence:

(SEQ ID NO: 11)
VSKGEELFTGVVPILVELDGDVNGHKFSVSGEGEGDATYGKLTLKFICT
TGKLPVPWPTLVTTLTYGVQCFSRYPDHMKQHDFFKSAMPEGYVQERTI
FFKDDGNYKTRAEVKFEGDTLVNRIELKGIDFKEDGNILGHKLEYNYNS
HNVYIMADKQKNGIKVNFKIRHNIEDGSVQLADHYQQNTPIGDGPVLLP
DNHYLSTQSKLSKDPNEKRDHMVLLEFVTAAGITLGMDELYK

### Example 4d: Transfection of Bifunctional Molecule

The Ibrutinib-conjugated ASOs described in Example 4a (shown in Table 5 below) were co-transfected into human cells along with the plasmid expressing the target Renilla luciferase described in example 4b, as well as the plasmid expressing BTK-YTHDF1 effector protein described example 4c.

A 96-well cell culture plate with 70% confluent HEK293T cells was transfected with the 50 nanograms of the target luciferase plasmid and 100 nanograms of the plasmid expressing the BTK-YTHDF1 effector, using Lipofectamine 2000 (Thermo Fisher Scientific) according to the manufacturer's instruction. After 24 hours, targeting (test) and non-targeting (control) ASOi were transfected separately into the cells at the final concentration of 100 nM using Lipofectamine RNaiMax (Thermo Fisher Scientific) according to the manufacturer's instruction. For each condition, cells were allowed to recover and are subsequently analyzed 48 hours after the transfection of ASOis.

### Example 4e: Measuring Protein Expression by Luciferase Activity

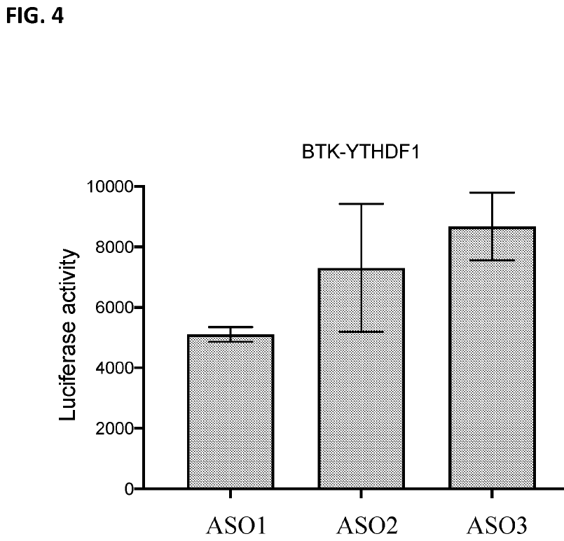

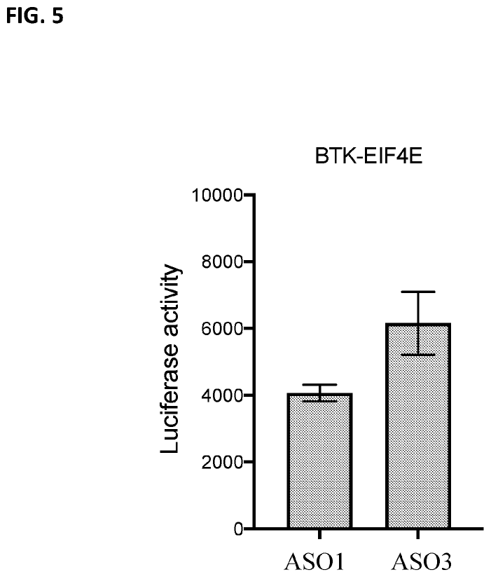

Pierce Renilla Luciferase Glow Assay Kit (Thermo Fisher Scientific) was used for measuring the luciferase activity corresponding to protein expression in each condition, according to manufacturer's instruction. The luminescence was measured and quantified by a GloMax plate reader and its integrated software (Promega Corp.), according to manufacturer's instruction (FIG. 4). Similar results were found when YTHDF1 was replaced with another effector, EIF4E (FIG. 5).

TABLE 5
Name, target, and sequence of ASOs used in examples related to
enhancement of translation. The effectors paired with each ASO are included in
the last column.

| | | | | | Effector(s) |
|---|---|---|---|---|---|
| ASO | | | Genomic | Targeted region | increasing |
| number | Target | Sequence | Coordinates | on transcript | translation |
| ASO1 | Non- | AGAGGTGGCGTG | None | NA | YTHDF1, EIF4E |
| | targeting | GTAG (SEQ ID | | | |
| | control | NO: 5) | | | |
| | (scramble) | | | | |
| ASO2 | Rluc | TGTGTCAGAAGAA | None | 5’-UTR | YTHDF1, EIF4E |
| | | TCAAGC (SEQ ID | | | |
| | | NO: 6) | | | |
| ASO3 | Rluc | TTCTGCAGCTTAA | None | 5’-UTR | YTHDF1, EIF4E |
| | | GTTCGA (SEQ ID | | | |
| | | NO: 7) | | | |

What is claimed is:

1. A method of increasing translation of a target ribonucleic acid (RNA) in a cell comprising:
administering to the cell a synthetic bifunctional molecule comprising:
a first domain comprising an antisense oligonucleotide (ASO) or a first small molecule, wherein the first domain specifically binds to a RNA sequence of the target RNA;
a second domain comprising a second small molecule or an aptamer, wherein the second domain specifically binds to a target polypeptide; and
a linker that conjugates the first domain to the second domain,
wherein the target polypeptide promotes, boosts, or increases translation of the target RNA in the cell.

2. The method of claim 1, wherein the target polypeptide is a target protein.

3. The method of any one of the preceding claims, wherein the first domain comprises the ASO.

4. The method of any one of the preceding claims, wherein the first domain comprises the ASO, and the ASO comprises one or more locked nucleic acids (LNA), one or more modified nucleobases, or a combination thereof.

5. The method of any one of the preceding claims, wherein the first domain comprises the ASO, and the ASO comprises a 5′ locked terminal nucleotide, a 3′ locked terminal nucleotide, or a 5′ and a 3′ locked terminal nucleotide.

6. The method of any one of the preceding claims, wherein the first domain comprises the ASO, and the ASO comprises a locked nucleotide at an internal position in the ASO.

7. The method of any one of the preceding claims, wherein the first domain comprises the ASO, and the ASO comprises a sequence comprising 30% to 60% GC content.

8. The method of any one of the preceding claims, wherein the first domain comprises the ASO, and the ASO comprises a length of 8 to 30 nucleotides.

9. The method of any one of the preceding claims, wherein the first domain comprises the ASO, and the ASO binds to Renilla Luciferase (Rluc) RNA.

10. The method of any one of the preceding claims, wherein the first domain comprises the ASO, the linker is conjugated at a 5′ end or a 3′ end of the ASO.

11. The method of any one of the preceding claims, wherein the cell is a human cell.

12. The method of claim 1 or 2, wherein the first domain comprises the first small molecule

13. The method of any one of the preceding claims, wherein the second domain comprises the second small molecule.

14. The method of claim 13, wherein the small molecule is an organic compound having a molecular weight of 900 daltons or less.

15. The method of claim 13, wherein the second small molecule comprises Ibrutinib or Ibrutinib-MPEA.

16. The method of any one of claims 1-12, wherein the second domain comprises the aptamer.

17. The method of any one of the preceding claims, wherein the linker comprises:

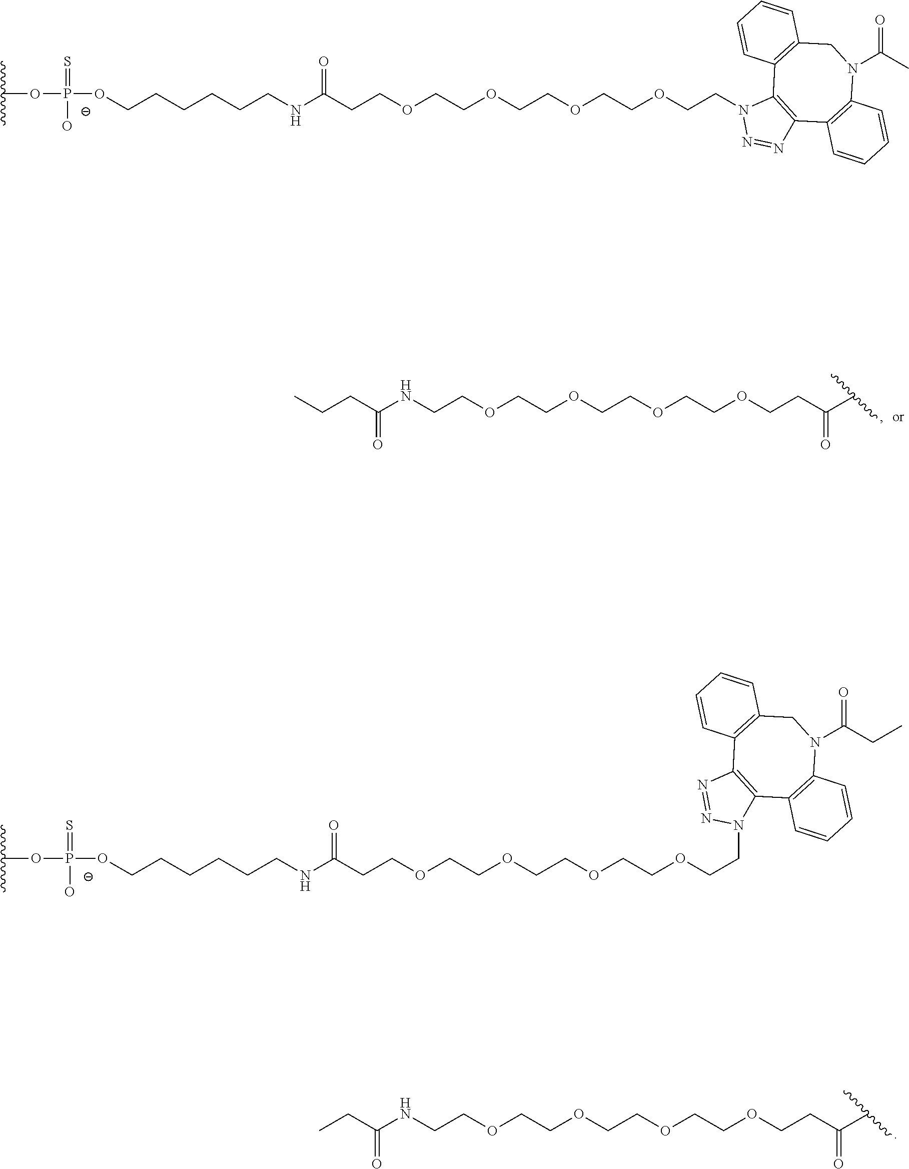

18. The method of any one of the preceding claims, wherein the target ribonucleic acid is a nuclear RNA or a cytoplasmic RNA.

19. The method of claim 18, wherein the nuclear RNA or the cytoplasmic RNA is a long noncoding RNA (lncRNA), pre-mRNA, mRNA, microRNA, enhancer RNA, transcribed RNA, nascent RNA, chromosome-enriched RNA, ribosomal RNA, membrane enriched RNA, or mitochondrial RNA.

20. The method of any one of the preceding claims, wherein a subcellular localization of the target RNA is selected from the group consisting of nucleus, cytoplasm, Golgi, endoplasmic reticulum, vacuole, lysosome, and mitochondrion.

21. The method of any one of the preceding claims, wherein the target RNA is located in an intron, an exon, a 5′ UTR, or a 3′ UTR of the target RNA.

22. The method of any one of the preceding claims, wherein the target polypeptide comprises EIF4E.

23. The method of any one of the preceding claims, wherein the target polypeptide comprises YTHDF1.

24. The method of any one of the preceding claims, wherein the target polypeptide is an endogenous polypeptide.

25. The method of any one of the preceding claims, wherein the target polypeptide is an intracellular polypeptide.

26. The method of any one of the preceding claims, wherein the target polypeptide is an enzyme or a regulatory protein.

27. The method of any one of the preceding claims, wherein the RNA is associated with a disease or disorder.

28. The method of any one of the preceding claims, wherein the RNA is associated with a tumor suppressor gene or haploinsufficiency gene.

29. A synthetic bifunctional molecule for increasing translation of a target ribonucleic acid (RNA) in a cell, the synthetic bifunctional molecule comprising:
a first domain comprising a first small molecule or an antisense oligonucleotide (ASO), wherein the first domain specifically binds to an RNA sequence of the target RNA;
a second domain comprising a second small molecule or an aptamer, wherein the second domain specifically binds to a target polypeptide; and
a linker that conjugates the first domain to the second domain,
wherein the target polypeptide promotes, boosts, or increases translation of the target RNA in the cell.

30. The method of claim 29, wherein the target polypeptide is a target protein.

31. The method of claim 29 or 30, wherein the linker comprises

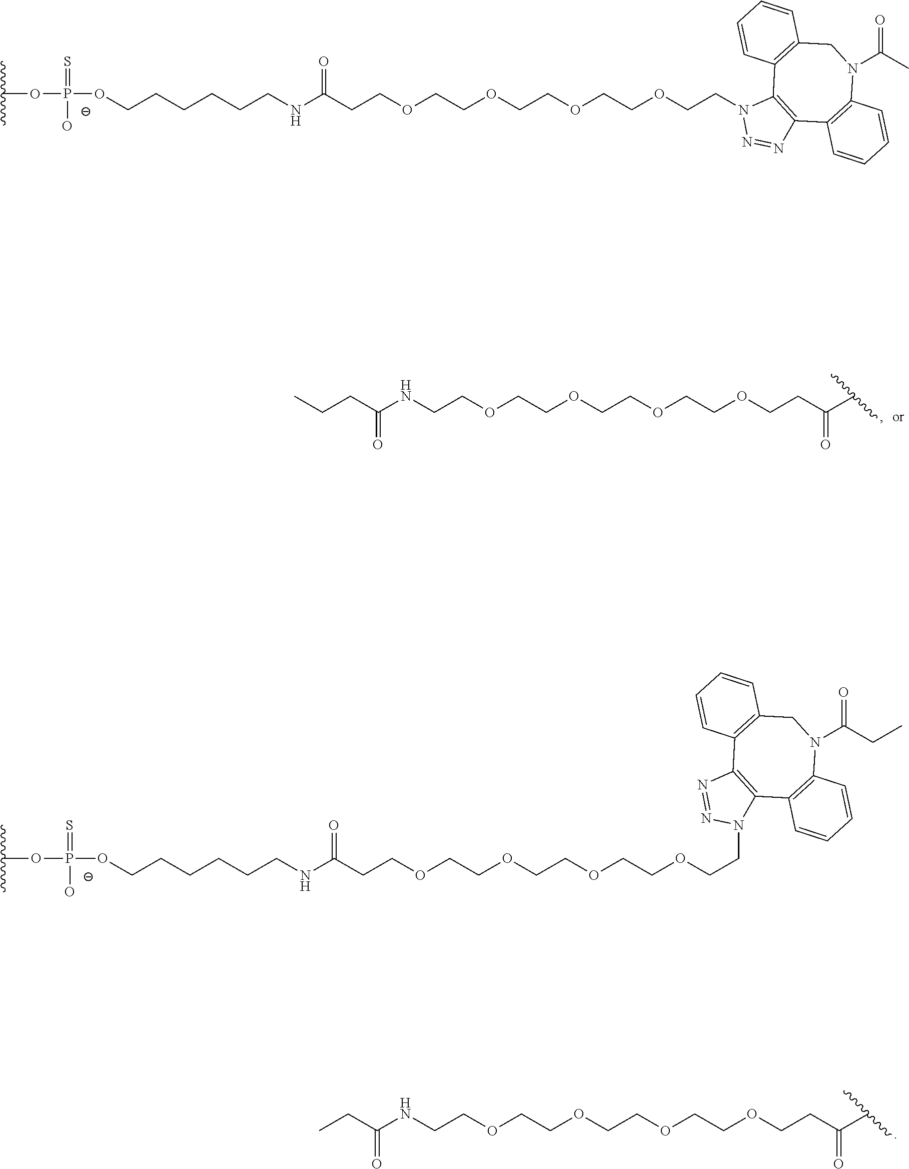

32. The method of any one of claims 29-31, wherein the target polypeptide is YTHDF1 or EIF4E.


SEQUENCE LISTING (11 sequences)

SEQ ID NO: 1 - DNA, 16 bp, Artificial Sequence
  antisense oligonucleotide 1 (ASO 1)
agaggtggcgtggtag

SEQ ID NO: 2 - DNA, 19 bp, Artificial Sequence
  ASO2
tgtgtcagaagaatcaagc

SEQ ID NO: 3 - DNA, 19 bp, Artificial Sequence
  ASO3
ttctgcagcttaagttcga

SEQ ID NO: 4 - RNA, 16 nt, Artificial Sequence
  Synthetic Sequence
cguuaacuaggcuuua

SEQ ID NO: 5 - RNA, 169 nt, Artificial Sequence
  Cy5-labeled IVT RNA
ccuugaaauccaugacgcagggagaauugcgucauuuaaagccuaguuaacgcauuuacuaaacgcagacgaaaauggaaagauuaauugggagugguaggaugaaacaauuuggagaagauagaaguuugaaguggaaaacuggaagacagaaguacgggaaggcgaa

SEQ ID NO: 6 - RNA, 16 nt, Artificial Sequence
  non-complementary RNA oligo
agagguggcgugguag

SEQ ID NO: 7 - protein, 278 aa, Artificial Sequence
  BTK protein
KNAPSTAGLGYGSWEIDPKDLTFLKELGTGQFGVVKYGKWRGQYDVAIKMIKEGSMSEDEFIEEAKVMMNLSHEKLVQLYGVCTKQRPIFIITEYMANGCLLNYLREMRHRFQTQQLLEMCKDVCEAMEYLESKQFLHRDLAARNCLVNDQGVVKVSDFGLSRYVLDDEYTSSVGSKFPVRWSPPEVLMYSKFSSKSDIWAFGVLMWEIYSLGKMPYERFTNSETAEHIAQGLRLYRPHLASEKVYTIMYSCWHEKADERPTFKILLSNILDVMDEES

SEQ ID NO: 8 - protein, 217 aa, Artificial Sequence [sequence omitted]

SEQ ID NO: 9 - protein, 559 aa, Artificial Sequence
  YTHDF1 protein
MSATSVDTQRTKGQDNKVQNGSLHQKDTVHDNDFEPYLTGQSNQSNSYPSMSDPYLSSYYPPSIGFPYSLNEAPWSTAGDPPIPYLTTYGQLSNGDHHFMHDAVFGQPGGLGNNIYQHRFNFFPENPAFSAWGTSGSQGQQTQSSAYGSSYTYPPSSLGGTVVDGQPGFHSDTLSKAPGMNSLEQGMVGLKIGDVSSSAVKTVGSVVSSVALTGVLSGNGGTNVNMPVSKPTSWAAIASKPAKPQPKMKTKSGPVMGGGLPPPPIKHNMDIGTWDNKGPVPKAPVPQQAPSPQAAPQPQQVAQPLPAQPPALAQPQYQSPQQPPQTRWVAPRNRNAAFGQSGGAGSDSNSPGNVQPNSAPSVESHPVLEKLKAAHSYNPKEFEWNLKSGRVFIIKSYSEDDIHRSIKYSIWCSTEHGNKRLDSAFRCMSSKGPVYLLFSVNGSGHFCGVAEMKSPVDYGTSAGVWSQDKWKGKFDVQWIFVKDVPNNQLRHIRLENNDNKPVTNSRDTQEVPLEKAKQVLKIISSYKHTTSIFDDFAHYEKRQEEEEVVRKERQSRNKQ

SEQ ID NO: 10 - protein, 18 aa, Artificial Sequence
  T2A self-cleaving peptide
EGRGSLLTCGDVEENPGP

SEQ ID NO: 11 - protein, 238 aa, Artificial Sequence
  monomeric enhanced fluorescence protein (mEGFP) protein
VSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSKLSKDPNEKRDHMVLLEFVTAAGITLGMDELYK